(19) United States
(12) Patent Application Publication
(10) Pub. No.: US 2023/0381298 A1
(43) Pub. Date: Nov. 30, 2023

(54) HERPESVIRUS POLYEPITOPE VACCINES

(71) Applicant: The Council of the Queensland Institute of Medical Research, QLD (AU)

(72) Inventors: Rajiv Khanna, Herston (AU); Dasari Vijayendra, The Gap (AU)

(21) Appl. No.: 18/030,892

(22) PCT Filed: Oct. 6, 2021
(86) PCT No.: PCT/IB21/00689

§ 371 Date: Apr. 7, 2023

Related U.S. Application Data
(60) Provisional application No. 63/088,766, filed on Oct. 7, 2020.

(51) Int. Cl. A61K 39/245; C07K 14/005; A61K 39/295; A61K 39/39; A61P 31/22; A61K 39/00; C12N 5/0783

(52) U.S. Cl. CPC A61K 39/245; C07K 14/005; A61K 39/295; A61K 39/39; A61P 31/22; A61K 39/4622; A61K 39/4634; A61K 39/464838; C12N 5/0638; A61K 2039/70; A61K 2039/572; C07K 2319/40; C07K 2319/50; A61K 2039/55561; A61K 2039/575; C12N 2710/16222; C12N 2501/998

(57) ABSTRACT

Provided herein are compositions and methods comprising immunogenic polypeptides related to the prevention and treatment of Epstein Ban vims infection and related pathologies.

### RELATED APPLICATIONS

This application claims the benefit of priority to U.S. Provisional Patent Application Ser. No. 63/088,766 filed Oct. 7, 2020, which is incorporated by reference herein in its entirety.

### BACKGROUND

Herpesviruses represent a large and near ubiquitous family of eukaryotic viruses associated with a variety of animal and human diseases. Herpesviridae share several common structures, e.g., double-stranded, linear DNA genomes, and a virion comprising an icosahedral capsid, which is itself wrapped in a layer of viral tegument and a lipid bilayer (the viral envelope). In addition, herpesviruses comprise characteristic and highly conserved glycoproteins, carried on the lipid bilayer envelope of the herpesvirus virion. At least some of these glycoproteins play a role in the initial attachment of virus to the cell surface and subsequent penetration into cells.

Epstein-Barr virus (EBV) is an oncogenic gamma human herpesvirus, infecting>95% of adults worldwide. It is considered one of the most transforming tumor viruses in humans and the only one that can readily immortalize human B cells into indefinitely growing lymphoblastoid cell lines (LCLs) in vitro. Primary EBV infection is usually acquired during infancy and childhood, through oral secretions by infecting resting B cells in the oropharynx or epithelial cells (Moss, et al. (2001). “The immunology of Epstein-Barr virus infection.” Philos Trans R Soc Lond B Biol Sci. 356(1408): 475-488). Following primary infection EBV establishes life-long latency through its potent transforming capacity of B cells, and may be asymptomatic. However, delayed primary infection can lead to a symptomatic disease known as acute infectious mononucleosis (IM), also known as glandular fever, in 50-70% of adolescents or young adults (Macsween, et al. (2003). “Epstein-Barr virus-recent advances.” Lancet Infect Dis. 3(3): 131-140; Balfour et al. (2013). “Behavioral, virologic, and immunologic factors associated with acquisition and severity of primary Epstein-Barr virus infection in university students.” J Infect Dis. 207(1): 80-88). The vast majority of cases are self-limiting with an excellent prognosis, but can cause significant morbidity in some individuals. For example, EBV infection carries significant health risks for immunocompromised or immunosuppressed individuals through reactivation of latent virus or reinfection. EBV is a prominent cause of lymphoproliferative diseases in solid organ or hematopoietic stem cell transplant patients (Shannon-Lowe, et al. (2017). “Epstein-Barr virus-associated lymphomas.” Philos Trans R Soc Lond B Biol Sci. 372(1732)). Furthermore, EBV has been associated with epithelial-, lymphocyte-, and smooth muscle-derived tumors in humans. Some of the most prominent EBV associated cancers include Burkitt's lymphoma (BL), diffuse large B cell lymphoma (DLBCL), Hodgkin's lymphoma (HL), oral hairy leukoplakia (OHL), nasopharyngeal carcinoma (NPC), gastric carcinoma (GC), plasmablastic lymphoma and primary effusion lymphoma. Each year approximately 200000 new cases of all malignancies in humans are linked with EBV worldwide (Cohen, et al. (2011). “Epstein-Barr virus: an important vaccine target for cancer prevention.” Sci Transl Med. 3(107): 107fs107). EBV is also strongly associated with autoimmune disorders, such as multiple sclerosis (MS), a chronic neuro-inflammatory condition of the central nervous system (Nielsen, et al. (2007). “Multiple sclerosis after infectious mononucleosis.” Arch Neurol. 64(1): 72-75; Ascherio, et al. (2012). “The initiation and prevention of multiple sclerosis.” Nat Rev Neurol. 8(11): 602-612), and rheumatoid arthritis. In rare cases, chronic active Epstein-Barr virus infection (CAEBV) may develop as a complication of infection, wherein the virus remains ‘active’ and the symptoms of an EBV infection never fully resolve. Recent studies have shown that a history of EBV-associated IM has been reported to confer an augmented risk of MS, HL in young adults, and NPC. EBV is associated with an estimated 143,000 deaths from cancer worldwide every year and there are around 2.5 million MS patients worldwide (Gm, et al. (2017). “Cutaneous EBV-related lymphoproliferative disorders.” Semin Diagn Pathol. 34(1): 60-75). Indeed, the National Institutes of health has designated EBV as a significant target for cancer prevention, thus both prophylactic and/or therapeutic strategies are required for limiting and/or prevention of EBV-associated disease.

Treatment options for EBV infection, particularly in immunocompromised individuals, are limited as current antiviral drugs are not considered effective against EBV. Preemptive and first-line therapy in patients with high risk for EBV-PTLD, for example, include B-cell depletion by use of rituximab. Use of purified plasma immunoglobulin (IGIV) and adoptive transfer immunotherapy have showed some success, but because such products are derived from human plasma they are difficult to produce in large quantities and their use carries the risk of the transmission of infectious disease.

Over the years, despite considerable efforts towards the development of a vaccine for EBV-associated diseases, no vaccine has been approved for prevention of EBV infection or EBV-associated cancers. Recent attempts to develop an EBV vaccine have proven unsuccessful. Previous prophylactic vaccine strategies were designed to target either neutralizing antibody responses or CD8⁺ T cell responses (Dasari, et al. (2017). “Designing an effective vaccine to prevent Epstein-Barr virus-associated diseases: challenges and opportunities.” Expert Rev Vaccines. 16(4): 377-390). Unfortunately, though such EBV vaccines were able to reduce the rate of IM, they were unable to prevent asymptomatic infection (Dasari, et al. (2019). “Prophylactic and therapeutic strategies for Epstein-Barr virus-associated diseases: emerging strategies for clinical development.” Expert Rev Vaccines. 18(5): 457-474). These EBV vaccine strategies have assessed EBV envelope glycoproteins, such as 350/220 (gp350), B (gB), H (gH), L (gL), the EBV gH/gL complex, as potential targets. However, it has been proposed that in order to elicit a protective, CD8 cytotoxic T cell response, viral antigens must be delivered in nucleic acid form (e.g., using a viral vector delivery system) rather than as exogenously-delivered proteins, so that the expressed polypeptide is properly processed and presented to T cells (Koup and Douek. (2012) “Vaccine Design for CD8 T Lymphocyte Responses.” Cold Spring Harb Perspect Med. 2011 September; 1(1): a007252.)

The majority of vaccine delivery platforms, in particular live-attenuated vaccines and viral vector based vaccines, have raised several regulatory concerns safety issues in children, immunocompromised patients and pregnant women. Thus, there is a great need for new and improved methods and compositions for the treatment of EBV and EBV-associated cancers and diseases.

### SUMMARY

Provided herein are immunogenic polypeptides, compositions, and methods related to the development of herpesvirus-specific prophylactic and/or therapeutic immunotherapy based on T cell epitopes (e.g., EBV epitopes) that are recognized by cytotoxic T cells (CTLs) and can be employed in the prevention and/or treatment of a herpesvirus infection, and/or cancer (e.g., a cancer expressing an EBV antigen provided herein). The immunogenic polypeptides contemplated herein may comprise amino acid sequences of each of a plurality of cytotoxic T-cell (CTL) epitopes from herpesvirus antigens. In some such embodiments, the polyepitope protein further comprises proteasome liberation amino acids or amino acid sequences between at least two of said plurality of CTL epitopes. Such polyepitope proteins are capable of eliciting a CTL response upon administration to a subject as an exogenous polypeptide. Preferably, the polypeptide comprises at least one of the CTL epitope amino acid sequences set forth in Table 1.

In certain aspects, provided herein are compositions (e.g., prophylactic or therapeutic compositions, including vaccine compositions) containing a polypeptide comprising one or more of the EBV epitopes described herein (e.g., EBV epitopes listed in Table 1) and/or a nucleic acid encoding such a polypeptide, as well as methods of treating and/or preventing EBV infection and/or associated disease (e.g., EBV-associated cancer or autoimmune disease) by administering such compositions to a subject. In some embodiments, the polypeptide is not a full-length EBV polypeptide. For example, the polypeptide may contain no more than 15, 20, 25, 30, 35 or 40 contiguous amino acids of a full-length EBV polypeptide. In some embodiments, the polypeptide consists, or consists essentially of, an EBV epitope described herein. In certain embodiments, the polypeptide is no more than 15, 20, 25, 30, 35 or 40 amino acids in length. In some embodiments, the composition further comprises an adjuvant.

In some aspects of the invention, provided herein is a prophylactic or therapeutic composition for eliciting an immunogenic response in a subject against a herpesvirus. Such compositions may comprise an immunogenic polypeptide as described herein, e.g., an immunogenic polypeptide comprising amino acid sequences derived from each of a plurality of cytotoxic T-cell (CTL) epitopes, wherein the polypeptide comprises at least one of the amino acid sequences set forth in SEQ ID NOs. 1 to 20, or any combination thereof. Preferably, said compositions further comprise at least one herpesvirus glycoprotein (e.g., gp350, gB, gH, gL, gHgL complex, gp42, any fragment thereof, or any combination thereof; and preferably gp350). In some such embodiments, the composition comprises at least one adjuvant.

Aspects of the invention, as disclosed herein, include multivalent EBV vaccines comprising i) an immunogenic polypeptide comprising an amino acid sequence as set forth in SEQ ID NO. 21; ii) at least one EBV glycoprotein; and iii) at least one adjuvant.

In some aspects, provided herein are nucleic acids comprising a sequence encoding one or more of the peptides provided herein. In some embodiments, the sequence encoding one or more of the peptides provided herein is operably linked to one or more regulatory sequences. In some embodiments, the nucleic acid is an expression vector. In some embodiments, the nucleic acid is an adenoviral vector.

In some aspects, provided herein are pharmaceutical compositions comprising the EBV peptides, CTLs, APCs, nucleic acids, and/or antigen-binding molecules described herein and a pharmaceutical acceptable carrier. In some embodiments, provided herein are methods for treating and/or preventing EBV infection and/or cancer in a subject by administering a pharmaceutical composition provided herein.

In further aspects, provided herein are methods for generating a prophylactic or therapeutic treatment for herpesvirus infection (e.g., EBV infection) comprising combining an isolated immunogenic polypeptide, at least one herpesvirus glycoprotein, at least one adjuvant comprising a TLR agonist, and a pharmaceutically acceptable excipient, in a formulation suitable for administration to a subject; wherein the immunogenic polypeptide comprises at least one of the CTL epitope amino acid sequences set forth in SEQ ID NOs. 1 to 20, or any combination thereof. Preferably, the immunogenic polypeptide comprises the amino acid sequence set forth in SEQ ID NO. 21.

In certain aspects, provided herein are methods for prophylactically or therapeutically treating a herpesvirus infection (e.g., EBV infection) in a subject, comprising administering to the subject a composition comprising i) an immunogenic polypeptide comprising amino acid sequences derived from each of a plurality of cytotoxic T-cell (CTL) epitopes, wherein the polypeptide comprises at least one of the amino acid sequences set forth in SEQ ID NOs. 1 to 20, or any combination thereof ii) at least one herpesvirus glycoprotein; and iii) an adjuvant. In preferred embodiments, the immunogenic polypeptide comprises the amino acid sequence set forth in SEQ ID NO. 21.

Also provided herein, in certain aspects, are methods of inducing proliferation of herpesvirus-specific CTLs, comprising bringing a sample comprising CTLs into contact with one or more peptides comprising at least one of the CTL epitope amino acid sequences set forth in SEQ ID NOs. 1 to 20, or any combination thereof.

In some aspects, provided herein is a method of identifying a subject suitable for a method of treatment provided herein (e.g., administration of CTLs, APCs, polypeptides, compositions, antibodies or nucleic acids described herein) comprising isolating a sample (e.g., a blood or tumor sample) from the subject and detecting the presence of an EBV epitope described herein or a nucleic acid encoding an EBV epitope described herein in the sample. In some embodiments, the EBV epitope is detected by contacting the sample with an antigen-binding molecule provided herein. In some embodiments, the method further comprises treating the identified subject according to a method of treatment provided herein.

### BRIEF DESCRIPTION OF THE DRAWINGS

FIG. 1 shows SDS-PAGE gel analysis of the EBV polyepitope protein expression, and protein purification. EBVpoly20PL-NH (EBVpoly) was expressed using an IPTG-inducible plasmid; after 4 hours of induction, expression levels were determined by SDS-PAGE analysis comparing un-induced and induced samples (A). EBVpoly protein solubility was assessed by SDS-PAGE analysis, comparing the supernatant and pellet fractions of cell lysate. EBVpoly protein was identified in pellet fractions in the form of inclusion bodies (IBs) (B). Cell pellets, comprising the IBs, were washed three times with TE buffer. The supernatant was analyzed to monitor protein loss (C). IBs were then solubilized and the pH of the solubilized protein was decreased to pH 7.0. prior to loading onto a fast protein liquid chromatography (FPLC) column. Flow through and column wash were assessed by SDS-PAGE analysis (D). Protein was eluted with a buffer containing 7.5 mM NaOH and 8M urea finally column was washed with 1M NaOH as indicated in the chromatogram (E). To maintain the purified protein in a buffer, 1 M tris pH 7.5 was added to the eluted protein to get a final concentration of tris buffer to 25 mM. The purified EBVpoly protein was dialyzed against 25 mM glycine pH 3.0 buffer and passed through Mustang E membrane to remove endotoxin contaminants, and then analysed on SDS-PAGE (F and G).

FIG. 2 shows the evaluation of EBVpoly protein immunogenicity in vitro, using intracellular cytokine staining (ICS) assay. PBMC from six different HLA-mapped healthy donors were stimulated with EBVpoly protein and cultured for 14 days prior to cytokine profile analysis by ICS.

FIG. 3 presents a schematic representation of the experimental design for evaluating the immunogenicity of EBV vaccine formulations comprising amphCpG7909 or CpG7909 in human HLA B35, A2, A24 and B8 transgenic mice. Four vaccine formulations were prepared, i.e., 1. amphCpG7909/EBVpoly/EBV gp350 (AmpCpG7909V); 2. soluble CpG7909/EBVpoly/EBV gp350 (CpG7909V); 3. amphCpG7909 alone (AmpCpG7909C); and 4. soluble CpG7909 alone (CpG7909C). All the cohorts of human HLA transgenic mice were immunized subcutaneously at each side of the tail base in 50 μL (100 μL total) on day 0, and received booster injections on days 21 and 42, with blood samples taken prior to each booster shot for analysis. Mice were sacrificed on day 49 and blood, lymph node, and spleen were harvested for analysis.

FIG. 4 shows the evaluation of ex vivo and memory EBVpoly-specific CD8⁺ T cell responses in splenocytes. Splenocytes suspensions were prepared from harvested (day 49) spleen and stimulated separately with HLA B35 (HPV and LPE), HLA A2 (CLG and GLC), HLA A24 (TYG and PYL) and HLA B8 (FLR and RAK) restricted peptides in the presence of golgi plug and golgi stop. To determine the memory response, cell suspensions of splenocytes were in vitro stimulated with EBVpoly peptides as mentioned above. Cells were cultured for 10 days in the presence of IL2. T cell specificity was assessed using ICS assay. The Bar graphs represents the ex vivo (A) and memory (B) mean T-cell responses quantified as a percentage of IFNγ⁺ of CD8⁺ T cell responses to EBV vaccine formulated with amphCpG7909 or CpG7909 or to control groups (adjuvant alone) in human HLA B35, A2, A24 and B8 transgenic mice. The Pie charts represents total percentage of ex vivo (top panel) and memory (bottom panel) EBVpoly-specific CD8⁺ T cells producing any combination of IFN-γ, TNF and/or IL2 (C and D) in human HLA B35, A2, A24 and B8 transgenic mice. Error bars represent the mean±SEM *, P<0.05; **, P<ns=not significant (determined by the student t test).

FIG. 5 shows the evaluation of ex vivo and memory EBV gp350-specific CD4⁺ T cell responses in splenocytes. To assess the ex vivo gp350-specific CD4⁺ T cell responses, splenocytes suspension was stimulated with PepMix™ in the presence of golgi plug and golgi stop. To determine the EBV gp350-specific memory CD4⁺ T cell responses, on day 49 splenocytes were in vitro stimulated with PepMix™ EBV, to expand gp350-specific CD4⁺ and CD8⁺ T cells for 10 days. Cells were cultured for 10 days in the presence of IL2 and were subsequently stimulated with PepMix™ to assess their ability to produce IFN-γ alone or IFN-γ, TNF and IL2. Ex vivo (tope panel) and memory (bottom panel) mean T-cell responses are quantified as a percentage of IFNγ⁺ of CD4⁺ T cell responses to EBV vaccine formulated with amphCpG7909 or CpG7909, and to control groups (adjuvants alone) in human HLA B35, A2, A24 and B8 transgenic mice (A and B). The pie chart represents total percentage of ex vivo (top panel) and memory (bottom panel) EBV gp350-specific CD4⁺ T cells producing any combination of IFN-γ, TNF and/or IL2 (C and D). Error bars represent the mean±SEM *, P<0.05; **, P<***, P<0.001; ns=not significant (determined by the student t test).

FIG. 6 shows evaluation of EBV gp350-specific CD8⁺ T cell responses following in vitro stimulation. Day 49 splenocytes were in vitro stimulated with PepMix™ EBV, to expand gp350-specific CD8⁺ T cells for 10 days, and were subsequently stimulated with PepMix™ EBV in the presence of golgi plug and golgi stop. The mean T-cell responses were quantified as a percentage of IFN-γ producing CD8⁺ T cell responses in human HLA B35 and A24 transgenic mice immunized with amphCpG7909 or CpG7909 EBV vaccine formulation or control groups (adjuvant alone). The bar graphs show the mean CD8⁺ T cell responses (i.e., IFN-γ production) in immunized HLA B35 and A24 mice (A and B). The pie charts show EBV gp350-specific CD8⁺ T cell producing any combination of IFN-γ, TNF and/or IL2 (C and D). Error bars represent the mean **, P<0.01; ns=not significant (determined by the student t test).

FIG. 7 shows the EBV-specific CD8⁺ and CD4⁺ T cell responses in inguinal lymph nodes. Single cell suspensions prepared from day-49 inguinal lymph nodes obtained from human HLA B35 and A2 transgenic mice and cells were stimulated with HLA B35 (HPV and LPE) or HLA A2 (GLC and CLG) restricted epitopes, and then assessed for their ability to produce IFN-γ or IFN-γ, TNF and IL2. The mean T-cell responses of stimulated CD8⁺ T cells from mice HLA B35 and A2 immunized with amphCpG7909-EBV vaccine or soluble CpG7909-EBV vaccine, or control groups (adjuvant alone) is depicted in the bar graph (A and B). The pie charts show the percentage of EBVpoly-specific CD8⁺ T cells producing any combination of IFN-γ, TNF and/or IL2 (C and D). Similarly, gp350-specific CD4⁺ T cell responses were assessed in inguinal lymph node cells stimulated with PepMix™ EBV. The bar graphs show the mean T-cell responses (percentage of IFN-γ⁺-producing CD4⁺ T cell responses) for each formulation in HLA B35 and A2 mice (E and F). Representative pie charts show the total percentage of EBV gp350-specific CD4⁺ T cells producing any combination of IFN-γ, TNF and/or IL2 in HLA B35 and A2 mice (G and H). Error bars represent the mean±SEM *, P<0.05; **, P<0.01; ns=not significant (determined by the student t test).

FIG. 8 shows the EBV-specific CD8⁺ and CD4⁺ T cell responses in axillary lymph nodes. Single cell suspensions prepared from day-49 axillary lymph nodes obtained from human HLA B35 and A2 transgenic mice and cells were stimulated with HLA (HPV and LPE) or HLA A2 (GLC and CLG) restricted epitopes, and then assessed for their ability to produce IFN-γ or IFN-γ, TNF and IL2. The mean T-cell responses of stimulated CD8⁺ T cells from mice HLA B35 and A2 immunized with amphCpG7909-EBV vaccine or soluble CpG7909-EBV vaccine, or control groups (adjuvant alone) is depicted in the bar graph (A and B). The pie charts show the percentage of EBVpoly-specific CD8⁺ T cells producing any combination of IFN-γ, TNF and/or IL2 (C and D). Likewise, gp350-specific CD4⁺ T cell responses were assessed in axillary lymph node cells stimulated with PepMix™ EBV. The bar graphs show the mean T-cell responses (percentage of IFN-γ⁺-producing CD4⁺ T cell responses) for each formulation in HLA B35 and A2 mice (E and F). Representative pie charts show the total percentage of EBV gp350-specific CD4⁺ T cells producing any combination of IFN-γ, TNF and/or IL2 in HLA B35 and A2 mice (G). Error bars represent the mean±SEM *, P<0.05; **, P<0.01; ns=not significant (determined by the student t test).

FIG. 9 shows the assessment of EBV gp350-specific antibody secreting plasma and memory B cell responses induced by EBV vaccine formulated with amphCpG7909 or CpG7909 in human HLA B35, A2, A24 and B8 transgenic mice. Day-49 splenocytes were assessed for their ability to secrete EBVgp350-specific antibodies (frequency of antibody secreting B cells/3×10⁵ splenocytes) ex vivo, using ELISpot assay (A). The memory B cell response in splenocytes (2.5×10⁴) stimulated with R848 (resiquimod) and mouse recombinant IL2 was also analyzed to determine their ability to secrete gp350-specific antibodies (B). Error bars represent the mean±SEM *, P<0.05; **, P<0.01; ***, P<0.001, ****, P<0.0001 ns=not significant (determined by the student t test).

FIG. 10 shows assessment of EBV gp350-specific antibody responses induced by EBV vaccine formulated with amphCpG7909 or CpG7909 in human HLA B35, A2, A24 and B8 transgenic mice. The line graph shows EBV gp350-specific antibody titers in serum samples from the transgenic mice immunized with the amphCpG7909-EBV vaccine formulation, the soluble CpG7909-EBV vaccine formulation, or with adjuvant-alone controls on day 21, 28, 42 and 49.

FIG. 11 shows assessment of EBV gp350-specific antibody isotypes induced by EBV vaccine formulated with amphCpG7909 or CpG7909 in human HLA B35, A2, A24 and B8 transgenic mice. The bar graphs show EBV gp350-specific antibody isotypes, IgA, IgM, IgG1, IgG2a, IgG2b and IgG3 titers in serum samples from the transgenic mice immunized with amphCpG7909-EBV vaccine formulation, the soluble CpG7909-EBV vaccine formulation.

FIG. 12 shows the EBV gp350-specific neutralizing antibody responses induced by EBV vaccine formulated with amphCpG7909 or CpG7909 in human HLA B35, A2, A24 and B8 transgenic mice. Briefly, analysis was performed on pooled serum samples (days 21, 28, 42, and 49) to assess anti-EBV-neutralizing antibody responses using a B cell proliferation assay. The Bar graphs represent the 50% EBV-specific neutralizing antibody titers in human HLA B35, A2, A24 and B8 transgenic mice vaccinated with amphCpG7909-EBV vaccine formulation, soluble CpG7909-EBV vaccine formulation, or control (adjuvant-alone).

FIG. 13 presents a schematic representation of the experimental design for evaluating the immunogenicity of EBV vaccine formulations comprising CpG1018 in human HLA B35 transgenic mice. Two vaccine formulations were prepared, i.e., 1. CpG1018/EBVpoly/EBV gp350 (EBV vaccine); and 2. CpG1018 alone (placebo). The human HLA B35 transgenic mice were immunized subcutaneously at the tail base in 100 μL on day 0, and received booster injections on days 21 and 42, with blood samples taken prior to each booster shot for analysis. Mice were sacrificed on day 49 and blood and spleens were harvested for analysis.

FIG. 14 shows the evaluation of ex vivo and memory EBVpoly-specific CD8⁺ T cell responses in splenocytes. Splenocytes suspensions were prepared from harvested (day 49) spleen and stimulated with HLA B35 (HPV and LPE) peptides in the presence of golgi plug and golgi stop. To determine the memory response, cell suspensions of splenocytes were in vitro stimulated with HPV and LPE peptides. Cells were cultured for 10 days in the presence of IL2. T cell specificity was assessed using ICS assay. The Bar graphs represents the ex vivo (top panel) and memory (bottom panel) mean T-cell responses quantified as a percentage of IFNγ⁺ of CD8⁺ T cell responses to EBV vaccine formulated with CpG1018 or CpG1018 alone (placebo) in human HLA B35 transgenic mice (A and C). The representative FACS plots and pie charts represents total percentage of ex vivo and memory EBVpoly-specific CD8⁺ T cells producing any combination of IFN-γ, TNF and/or IL2 (B and D) in human HLA B35 transgenic mice. Error bars represent the mean±SEM *, P<0.05; **, P<0.01 (determined by the student t test).

FIG. 15 shows the evaluation of ex vivo and memory EBV gp350-specific CD4⁺ T cell responses in splenocytes. To assess the ex vivo gp350-specific CD4⁺ T cell responses, splenocytes suspension was stimulated with PepMix™ in the presence of golgi plug and golgi stop. To determine the EBV gp350-specific memory CD4⁺ T cell responses, on day 49 splenocytes were in vitro stimulated with PepMix™ EBV, to expand gp350-specific CD4⁺ and CD8⁺ T cells for 10 days. Cells were cultured for 10 days in the presence of IL2 and were subsequently stimulated with PepMix™ to assess their ability to produce IFN-γ alone or IFN-γ, TNF and IL2. Ex vivo (tope panel) and memory (memory) mean T-cell responses are quantified as a percentage of IFNγ⁺ of CD4⁺ T cell responses to EBV vaccine formulated with CpG1018 or CpG1018 alone (placebo) in human HLA B35 transgenic mice (A and C). The FACS plots and pie chart represents total percentage of ex vivo (top panel) and memory (bottom panel) EBV gp350-specific CD4⁺ T cells producing any combination of IFN-γ, TNF and/or IL2 (B and D). Error bars represent the mean±SEM *, P<0.05 (determined by the student t test).

FIG. 16 shows evaluation of EBV gp350-specific CD8⁺ T cell responses following in vitro stimulation. Day 49 splenocytes were in vitro stimulated with PepMix™ EBV, to expand gp350-specific CD8⁺ T cells for 10 days, and were subsequently stimulated with PepMix™ EBV in the presence of golgi plug and golgi stop. The mean T-cell responses were quantified as a percentage of IFN-γ producing CD8⁺ T cell responses in human HLA B35 transgenic mice immunized with EBV vaccine with CpG1018 or CpG1018 (placebo) formulations. The bar graphs show the mean CD8⁺ T cell responses (i.e., IFN-γ production) in immunized HLA B35 mice (A). The FACS plots and pie charts represent EBV gp350-specific CD8⁺ T cell producing any combination of IFN-γ, TNF and/or IL2 (B). Error bars represent the mean *, P<0.05 (determined by the student t test).

FIG. 17 shows the characterization of Germinal Center (GC) B, T_{FH} and EBV gp350-specific antibody secreting B cell responses induced by EBV vaccine formulated with CpG1018 or CpG1018 alone (placebo). To assess GC B cell responses, splenocytes were stained with PE conjugated anti-B220, FITC conjugated anti-GL7 and APC conjugated anti-CD95 (A). To assess T_{FH} cells, splenocytes were stained with PerCP conjugated anti-CD8, BV786 conjugated anti-CD4, CxCR5 and PD-1 surface markers (B). To assess gp350-specific antibody secreting B cells, day-49 splenocytes were assessed for their ability to secrete EBVgp350-specific antibodies (frequency of antibody secreting B cells/3×10⁵ splenocytes) ex vivo, using ELISpot assay (C). The memory B cell response in splenocytes (5×10⁵) stimulated with R848 (resiquimod) and mouse recombinant IL2 was also analyzed to determine their ability to secrete gp350-specific antibodies (D). Error bars represent the mean±SEM *, P<0.05; **, P<0.01; ***, P<0.001, ****, P<0.0001 ns=not significant (determined by the student t test).

FIG. 18 shows assessment of EBV gp350-specific antibody isotypes induced by EBV vaccine formulated with CpG1018 or CpG1018 alone. The line graph shows EBV gp350-specific antibody isotypes, IgA, IgM, IgG1, IgG2a, IgG2b and IgG3 titers in serum samples from the HLA B35 transgenic mice immunized with CpG1018 (V) or with CpG1018 adjuvant-alone (C) on day 21, 28, 42 and 49.

FIG. 19 shows the EBV gp350-specific neutralizing antibody responses induced by EBV vaccine formulated with CpG1018 in human HLA B35 transgenic mice. Briefly, analysis was performed on pooled serum samples (days 21, 28, 42, and 49) to assess anti-EBV-neutralizing antibody responses using a B cell proliferation assay. The Bar graphs represent the 50% EBV-specific neutralizing antibody titers in human HLA B35 transgenic mice vaccinated with EBV vaccine formulation with CpG1018 or control (adjuvant-alone) (A). Representative FACS plots show percentage of proliferating B cells in uninfected PBMC, PBMC infected with EBV virus, virus treated with serially diluted serum (1:2 and 1:512) from mice vaccinated with CpG1018-EBV vaccine formulation, or control (adjuvant-alone) (1:2) (B).

### DETAILED DESCRIPTION

### General

The primary strategy applied in EBV vaccine development has been to prevent primary infection and latency, thus preventing the development of EBV-associated malignancies. Some of these initial vaccine studies targeted the major viral glycoprotein gp350, as a neutralizing antibody target, because pre-existing antibodies provide a first line of defense against many viral pathogens. Multiple potent neutralizing antibodies targeting gp350 are present in infected human blood, and can prevent neonatal infection, making gp350 an attractive candidate in the development of EBV vaccines. However, in a phase I/II clinical trial in young adults, vaccination with soluble recombinant gp350 formulated with ASO4 did not prevent EBV infection (e.g., asymptomatic infection), although incidence of IM symptoms were reduced (Sokal, et al. (2007). “Recombinant gp350 vaccine for infectious mononucleosis: a phase 2, randomized, double-blind, placebo-controlled trial to evaluate the safety, immunogenicity, and efficacy of an Epstein-Barr virus vaccine in healthy young adults.” J Infect Dis. 196(12): 1749-1753; Dasari, et al. (2019). “Prophylactic and therapeutic strategies for Epstein-Barr virus-associated diseases: emerging strategies for clinical development.” Expert Rev Vaccines. 18(5): 457-474). A different vaccine formulation, tested in children awaiting kidney transplant, failed to protect subjects from PTLD (Rees, et al. (2009). “A phase I trial of Epstein-Barr virus gp350 vaccine for children with chronic kidney disease awaiting transplantation.” Transplantation. 88(8): 1025-1029). In yet another vaccine study, using HLA B0801 CD8⁺ T cell epitope from EBV latency protein (EBNA-3A), showed that vaccine was unable to prevent infection (Burrows, et al. (1990). “An Epstein-Barr virus-specific cytotoxic T-cell epitope present on A- and B-type transformants.” J Virol. 64(8): 3974-3976; Elliott, et al. (2008). “Phase I trial of a CD8⁺ T-cell peptide epitope-based vaccine for infectious mononucleosis.” J Virol. 82(3): 1448-1457). Thus, these observations raise questions about the type of immune response needed to be generated to improve EBV vaccine efficacy. Preclinical in vitro and in vivo models, and clinical observations suggest cytotoxic lymphocytes as the main immune compartment exerting immune control against infection. Vaccine formulations designed to induce both humoral and cellular (e.g., cytotoxic lymphocytes) responses should provide better protection than vaccines designed to induce either a humoral or cell-mediated response alone. The life cycle of EBV and its gene expression profile in its various associated diseases needs to be considered when selecting vaccine antigen(s) and inducing an optimal immune response.

Without being bound by any particular theory, a vaccine which can induce a broad repertoire of optimized virus-specific immune responses is likely to provide more effective protection against virus-associated pathogenesis. Disclosed herein is EBV gp350, and fragments thereof, comprising the gp350 extracellular domain. Said EBV glycoprotein, and fragments thereof, may act as a target for neutralizing antibody and CD4+ and CD8+ T cell responses. Also disclosed herein are peptides comprising at least one EBV T cell epitope. Preferably, such peptides are designed to encode multiple HLA class I restricted CD8⁺ T-cell epitopes (e.g., EBVpoly) from highly conserved immunodominant antigens (EBNA1, LMP2a, EBNA 3A, EBNA3B, EBNA3C, BMLF1, BZLF1, BRLF1) of EBV. What is more, a vaccine which can induce both humoral and cell-mediated immune response to a broad repertoire of EBV-specific antigens is likely to provide more effective protection. Thus, in certain aspects of the invention, to induce EBV-specific humoral and cell-mediated responses, described herein for the first time is a novel multivalent vaccine that comprises both an EBV gp350 peptide (or fragments thereof) and an EBV-epitope polyepitope polypeptide (e.g., EBVpoly). Such polypeptides, compositions, and methods related to the development of herpesvirus-specific prophylactic and/or therapeutic immunotherapy based on T cell epitopes (e.g., EBV epitopes) that are recognized by cytotoxic T cells (CTLs) as are disclosed herein, can be employed in the prevention and/or treatment of EBV infection, cancer (e.g., a cancer expressing an EBV antigen provided herein), and/or autoimmune diseases. In certain aspects, provided herein are compositions (e.g., therapeutic compositions, such as vaccine compositions) containing an immunogenic polypeptide comprising one or more of the EBV epitopes described herein (e.g., EBV epitopes listed in Table 1), nucleic acids encoding such a polypeptide, CTLs that recognize such a peptide, APCs presenting such peptides and/or antigen-binding molecules that bind specifically to such peptides, as well as methods of treating and/or preventing EBV infection, cancer, and/or an autoimmune disease by administering such compositions to a subject. In some embodiments, also provided herein are methods of identifying a subject suitable for treatment according to a method provided herein.

### Definitions

For convenience, certain terms employed in the specification, examples, and appended claims are collected here.

The articles “a” and “an” are used herein to refer to one or to more than one (i.e., to at least one) of the grammatical object of the article. By way of example, “an element” means one element or more than one element.

As used herein, the term “administering” means providing a pharmaceutical agent or composition to a subject, and includes, but is not limited to, administering by a medical professional and self-administering. Such an agent can contain, for example, peptide described herein, an antigen-presenting cell provided herein and/or a CTL provided herein.

As used herein, the term “subject” or “recipient” means a human or non-human animal selected for treatment or therapy.

“Treating” a disease in a subject or “treating” a subject having a disease, as used herein, refers to subjecting the subject to a pharmaceutical treatment, e.g., the administration of a drug, such that at least one symptom of the disease is decreased or prevented from worsening.

As used herein, a therapeutic that “prevents” a condition refers to a compound that, when administered to a statistical sample prior to the onset of the disorder or condition, reduces the occurrence of the disorder or condition in the treated sample relative to an untreated control sample, or delays the onset or reduces the severity of one or more symptoms of the disorder or condition relative to the untreated control sample.

As used herein, the phrase “pharmaceutically acceptable” refers to those agents, compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the phrase “pharmaceutically-acceptable carrier” means a pharmaceutically-acceptable material, composition or vehicle, such as a liquid or solid filler, diluent, excipient, or solvent encapsulating material, involved in carrying or transporting an agent from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

The terms “polynucleotide”, and “nucleic acid” are used interchangeably. They refer to a polymeric form of nucleotides of any length, either deoxyribonucleotides or ribonucleotides, or analogs thereof. Polynucleotides may have any three-dimensional structure, and may perform any function. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. A polynucleotide may be further modified, such as by conjugation with a labeling component. In all nucleic acid sequences provided herein, U nucleotides are interchangeable with T nucleotides.

The term “vector” refers to the means by which a nucleic acid can be propagated and/or transferred between organisms, cells, or cellular components. Vectors include plasmids, viruses, bacteriophage, pro-viruses, phagemids, transposons, and artificial chromosomes, and the like, that may or may not be able to replicate autonomously or integrate into a chromosome of a host cell.

### Peptides

Provided herein are peptides comprising herpesvirus epitopes that are recognized by cytotoxic T lymphocytes (CTLs) and that are useful in the prevention and/or treatment of herpesvirus infection (e.g., EBV infection), cancer (e.g., a cancer expressing an EBV epitope provided herein), and/or an autoimmune disease. In some aspects, provided herein are immunogenic polypeptides comprising at least one amino acid sequence of a cytotoxic T-cell (CTL) epitope from a herpesvirus antigens (e.g., and EBV antigen). In preferred embodiments, immunogenic polypeptides disclosed herein comprise the amino acid sequences of each of a plurality of cytotoxic T-cell (CTL) epitopes from herpesvirus antigens. Most preferably, such immunogenic polypeptides comprise HLA class I restricted CD8⁺ T-cell epitopes from highly conserved immunodominant antigens, such as EBNA1, EBNA3A, EBNA3B, EBNA3C, LMP2, LMP2a, BMLF1, BZLF1, or BRLF1. The epitopes may be restricted by any one of the HLA class I specificities selected from HLA A*03, HLA A11, HLA A*0201, HLA A*1101, HLA A*2301, HLA A*3002, HLA B27, HLA B35.08/B35.01, HLA B*44:0, HLA B57*03, HLA B*0702, HLA B*0801, HLA B*1501, HLA B*3501, HLA B*3508, HLA B*4001, HLA B*4402, HLA B*4402, HLA B*4403, HLA B*4405, HLA B*5301, HLA B*5701, or HLA B*5801. In certain embodiments, said epitopes are EBV epitopes listed in Table 1.

TABLE 1
Exemplary EBV epitopes

| Epitope Amino | | HLA | hEBV |
|---|---|---|---|
| Acid Sequence | Epitope Nucleotide Sequence | restriction | antigen |
| HPVGEADYFEYR | catccagttggtgaagcagactactttgaa | HLA B*3501, | EBNA1 |
| (SEQ ID NO. 1) | taccgt | HLA B*3508, | |
| | (SEQ ID NO. 22) | HLA B*5301 | |
| SSCSSCPLSKIAD | tcctcttgcagctcgtgtccgctgagcaag | HLA A11 | LMP2a |
| (SEQ ID NO. 2) | attgcagat | | |
| | (SEQ ID NO. 23) | | |
| RPPIFIRRLK | cgtccgccgatcttcatccgtcgtttgaaa | HLA B*0702 | EBNA 3A |
| (SEQ ID NO. 3) | (SEQ ID NO. 24) | | |
| FLRGRAYGLR | tttctgcgcggtcgcgcgtacggcttgcgt | HLA B*0801 | EBNA 3A |
| SEQ ID NO. 4) | (SEQ ID NO. 25) | | |
| GLCTLVAMLAD | ggtctgtgcaccctggtggccatgctggc | HLA A*0201 | BMLF1 |
| (SEQ ID NO. 5) | ggac | | |
| | (SEQ ID NO. 26) | | |
| EECDSELEIKRYK | gaggagtgtgatagcgagctcgaaatca | HLA-B*44:0 | BZLF1 |
| (SEQ ID NO. 6) | aacgctataag | | |
| | (SEQ ID NO. 27) | | |
| CLGGLLTMVAD | tgcctgggtggccttctgacgatggttgct | HLA A*0201 | LMP2a |
| (SEQ ID NO. 7) | gac | | |
| | (SEQ ID NO. 28) | | |
| RAKFKQLLR | cgtgcgaagtttaagcaactgctgcgc | HLA B*0801 | BZLF1 |
| (SEQ ID NO. 8) | (SEQ ID NO. 29) | | |
| ATIGTAMYKAD | gccaccattggtacggcaatgtataaagct | HLA A*1101 | BRLF1 |
| (SEQ ID NO. 9) | gac | | |
| | SEQ ID NO. 30) | | |
| TYGPVFMCLK | acctatggcccggttttcatgtgtctgaag | HLA A*2402 | LMP2a |
| (SEQ ID NO. 10) | (SEQ ID NO. 31) | | |
| LPEPLPQGQLTAYK | ctgccggagccgctgccgcagggtcaac | HLA | BZLF1 |
| (SEQ ID NO. 11) | tgaccgcatacaag | B35.08/B35.01 | |
| | (SEQ ID NO. 32) | | |
| IEDPPFNSLAD | attgaggacccgccgttcaatagcctggc | HLA B*4001 | LMP2a |
| (SEQ ID NO. 12) | ggac | | |
| | (SEQ ID NO. 33) | | |
| VSFIEFVGWK | gtgagcttcattgaatttgtcggctggaaa | HLA B*5701, | EBNA3B |
| (SEQ ID NO. 13) | (SEQ ID NO. 34) | HLA B57*03, | |
| | | HLA B*5801 | |
| EENLLDFVRFMGVK | gaagagaatttgctggacttcgtccgcttc | HLA B*4402, | EBNA3C |
| (SEQ ID NO. 14) | atgggcgtgaaa | HLA B*4405 | |
| | (SEQ ID NO. 35) | | |
| QNGALAINTFR | cagaacggtgctctggcaatcaacacgttt | HLA B*1501 | EBNA3C |
| (SEQ ID NO. 15) | cgt | | |
| | (SEQ ID NO. 36) | | |
| PYLFWLAAIR | ccgtacctgttctggctggcggccattcgt | HLA A*2301 | DNAse |
| SEQ ID NO. 16) | (SEQ ID NO. 37) | | |
| AYSSWMYSYAD | gcgtatagcagctggatgtacagctatgc | HLA A*3002 | IE-1 |
| (SEQ ID NO. 17) | cgat | | |
| | (SEQ ID NO. 38) | | |
| RVRAYTYSKAD | cgtgtccgcgcgtacacctactccaaagc | HLA A*03 | IE-1 |
| (SEQ ID NO. 18) | ggat | | |
| | (SEQ ID NO. 39) | | |
| RRIYDLIELR | cgtcgtatctacgatctgatcgagctgcgt | HLA B27 | IE-1 |
| SEQ ID NO. 19) | (SEQ ID NO. 40) | | |
| VEITPYKPTWAD | gttgaaattaccccgtataaacctacttgg | HLA B*4402, | pp65 |
| (SEQ ID NO. 20) | gcggat | HLA B*4403 | |
| | (SEQ ID NO. 41) | | |

Underlined amino acids show proteasome liberation sequences, which are optionally present as a portion of the EBV epitope.

In some embodiments, the immunogenic peptides provided herein are full length EBV polypeptides. In some embodiments, the peptides provided herein comprise less than 100, 90, 80, 70, 60, 50, 40, 30, 25, 20, 15 or 10 contiguous amino acids of the EBV viral polypeptide. In some embodiments, the peptides provided herein comprise two or more of the EBV epitopes listed in Table 1, that optionally possess or do not possess the identified proteasome liberation sequence. For example, in some embodiments, the peptide provided herein comprises two or more of the EBV epitopes listed in Table 1 connected by polypeptide linkers. By way of non-limiting example, such polyepitope peptide sequences may be designed in such a way that each epitope is joined by a linker that comprises, consists essentially of or consists of a proteasome liberation amino acid sequence (e.g., alanine and aspartic acid (AD) or lysine (K) or arginine (R)). In some embodiments, the peptide provided herein comprises at least 2, 3, 4, 5, 6, 7, 8, 9, 10, 11, 12, 13, 14, 15, 16, 17, 18, 19 or all of the epitopes listed in Table 1. In preferred embodiments, the immunogenic polypeptide of the invention comprises the amino acid sequence set forth in SEQ ID NO. 21. Examples of polyepitope polypeptides, methods of generating polyepitope polypeptides, and vectors encoding polyepitope polypeptides can be found in Dasari et al., Molecular Therapy—Methods & Clinical Development (2016) 3, 16058, which is hereby incorporated by reference in its entirety.

In certain aspects, provided herein are pools of immunogenic peptides comprising HLA class I and class II-restricted EBV peptide epitopes (e.g., epitopes listed in Tables 1) capable of inducing proliferation of peptide-specific T cells. In some embodiments, the pool of immunogenic peptides comprises at least 1, 2, 3, 4, 5, 6, 7, 8, 9, 10, 11, 12, 13, 14, 15, 16, 17, 18, 19 or all of the epitopes listed in Table 1 (e.g., at least 1, 2, 3, 4, 5, 6, 7, 8, 9, 10, 11, 12, 13, 14, 15, 16, 17, 18, 19, or 20 of the epitopes listed in Table 1), or combinations thereof. In preferred embodiments, the peptide pool comprises at least one EBV epitope set forth in Table 1, i.e., any one of the EBV epitopes set forth in SEQ ID Nos: 1-20, or any combination thereof. For example, the pool of immunogenic peptides may comprise at least 1, 2, 3, 4, 5, 6, 7, 8, 9, 10, 11, 12, 13, 14, 15, 16, 17, 18, 19, or all 20 of the epitopes encoded by the amino acid sequences set forth in SEQ ID Nos: 1-20. Most preferably, such peptide pools comprise each of the EBV peptide epitope amino acid sequences set forth in in SEQ ID Nos: 1-20. The immunogenic peptides, and pools thereof, are capable of inducing proliferation of peptide-specific T cells (e.g., peptide-specific cytotoxic T-cells and/or CD4⁺ T cells).

In some embodiments, the sequence of the peptides comprise an EBV viral polypeptide sequence except for 1 or more (e.g., 1, 2, 3, 4, 5, 6, 7, 8, 9, 10 or more) conservative sequence modifications. As used herein, the term “conservative sequence modifications” is intended to refer to amino acid modifications that do not significantly affect or alter the interaction between a T-cell receptor (TCR) and a peptide containing the amino acid sequence presented on an major histocompatibility complex (MEW). Such conservative modifications include amino acid substitutions, additions (e.g., additions of amino acids to the N or C terminus of the peptide) and deletions (e.g., deletions of amino acids from the N or C terminus of the peptide). Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues of the peptides described herein can be replaced with other amino acid residues from the same side chain family and the altered peptide can be tested for retention of TCR binding using methods known in the art. Modifications can be introduced into an antibody by standard techniques known in the art, such as site-directed mutagenesis and PCR-mediated mutagenesis.

To determine the percent identity of two amino acid sequences or of two nucleic acid sequences, the sequences are aligned for optimal comparison purposes (e.g., gaps can be introduced in one or both of a first and a second amino acid or nucleic acid sequence for optimal alignment and non-identical sequences can be disregarded for comparison purposes). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences.

Also provided herein are chimeric or fusion proteins. As used herein, a “chimeric protein” or “fusion protein” comprises a peptide(s) provided herein (e.g., those comprising an epitope listed in Table 1) linked to a distinct peptide to which it is not linked in nature. For example, the distinct peptide can be fused to the N-terminus or C-terminus of the peptide either directly, through a peptide bond, or indirectly through a chemical linker. In some embodiments, the peptide of the provided herein is linked to polypeptides comprising other EBV epitopes. In some embodiments, the peptide provided herein is linked to peptides comprising epitopes from other viral and/or infectious diseases. In some embodiments, the peptide provided herein is linked to a peptide encoding a cancer-associated epitope.

A chimeric or fusion peptide provided herein can be produced by standard recombinant DNA techniques. For example, DNA fragments coding for the different peptide sequences can be ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. Similarly, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and re-amplified to generate a chimeric gene sequence (see, for example, Current Protocols in Molecular Biology, Ausubel et al., eds., John Wiley & Sons: 1992). Moreover, many expression vectors that already encode a fusion moiety are commercially available.

In some aspects, provided herein are cells that present a peptide described herein (e.g., a peptide comprising an epitope listed in Table 1). In some embodiments, the cell is a mammalian cell. The cell may be an antigen-presenting cell (APC) (e.g., an antigen presenting t-cell, a dendritic cell, a B cell, a macrophage or an artificial antigen presenting cell, such as aK562 cell). A cell presenting a peptide described herein can be produced by standard techniques known in the art. For example, a cell may be pulsed to encourage peptide uptake. In some embodiments, the cells are transfected with a nucleic acid encoding a peptide provided herein.

In some aspects, provided herein are methods of producing antigen-presenting cells (APCs), comprising pulsing a cell with the peptides described herein. Exemplary methods for producing antigen presenting cells can be found in WO2013088114, hereby incorporated in its entirety.

The peptides described herein can be isolated from cells or tissue sources by an appropriate purification scheme using standard protein purification techniques, can be produced by recombinant DNA techniques, and/or can be chemically synthesized using standard peptide synthesis techniques. The peptides described herein can be produced in prokaryotic or eukaryotic host cells by expression of nucleotides encoding a peptide(s) of the present invention. Alternatively, such peptides can be synthesized by chemical methods. Methods for expression of heterologous peptides in recombinant hosts, chemical synthesis of peptides, and in vitro translation are well known in the art and are described further in Maniatis et al., Molecular Cloning: A Laboratory Manual (1989), 2nd Ed., Cold Spring Harbor, N. Y.; Berger and Kimmel, Methods in Enzymology, Volume 152, Guide to Molecular Cloning Techniques (1987), Academic Press, Inc., San Diego, Calif.; Merrifield, J. (1969) J. Am. Chem. Soc. 91:501; Chaiken I. M. (1981) CRC Crit. Rev. Biochem. 11:255; Kaiser et al. (1989) Science 243:187; Merrifield, B. (1986) Science 232:342; Kent, S. B. H. (1988) Annu. Rev. Biochem. 57:957; and Offord, R. E. (1980) Semisynthetic Proteins, Wiley Publishing, which are incorporated herein by reference.

### Nucleic Acid Molecules

Provided herein are nucleic acid molecules that encode the peptides described herein. For example, and without limitation, provided herein is a nucleic acid encoding an immunogenic polypeptide, wherein the nucleic acid comprises at least one of the nucleic acid sequences set forth in SEQ ID NOs. 22-41. In certain embodiments, the nucleic acid comprises each of the nucleic acid sequences set forth in SEQ ID NOs. 22-41. In some such embodiments, the nucleic acid comprises the nucleic acid sequence set forth in SEQ ID NO. 42.

In some aspects, provided herein are methods of treating and/or preventing cancer (e.g., EBV-associated cancer), EBV infection, and/or an autoimmune disease by administering to a subject the nucleic acids disclosed herein. The nucleic acids may be present, for example, in whole cells, in a cell lysate, or isolated in a partially purified or substantially pure form.

In some embodiments, provided herein are vectors (e.g., a viral vector, such as an adenovirus based expression vector) that contain the nucleic acid molecules described herein. One type of vector is a “plasmid”, which refers to a circular double-stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication, episomal mammalian vectors). Other vectors (e.g., non-episomal mammalian vectors) can be integrated into the genome of a host cell upon introduction into the host cell, and thereby be replicated along with the host genome. Moreover, certain vectors are capable of directing the expression of genes. Such vectors are referred to herein as “recombinant expression vectors” (or simply, “expression vectors”). In some embodiments, provided herein are nucleic acids operable linked to one or more regulatory sequences (e.g., a promoter) in an expression vector. In some embodiments the cell transcribes the nucleic acid provided herein and thereby expresses an antibody, antigen-binding fragment thereof, or peptide described herein. The nucleic acid molecule can be integrated into the genome of the cell or it can be extrachromosomal.

In some embodiments, the nucleic acid provided herein is part of a vaccine. In some embodiments, the vaccine is delivered to a subject in a vector, including, but not limited to, a bacterial vector and/or a viral vector. Examples of bacterial vectors include, but are not limited to, Mycobacterium bovis (BCG), Salmonella Typhimurium ssp., Salmonella Typhi ssp., Clostridium sp. spores, Escherichia coli Nissle 1917, Escherichia coli K-12/LLO, Listeria monocytogenes, and Shigella flexneri. Examples of viral vectors include, but are not limited to, vaccinia, adenovirus, RNA viruses (replicons), and replication-defective like avipox, fowlpox, canarypox, MVA, and adenovirus.

In some embodiments, provided herein are cells that contain a nucleic acid described herein (e.g., a nucleic acid encoding an antibody, antigen binding fragment thereof or peptide described herein). The cell can be, for example, prokaryotic, eukaryotic, mammalian, avian, murine and/or human. In some embodiments, the cell is a mammalian cell. In some embodiments the cell is an APC (e.g. an antigen presenting T cell, a dendritic cell, a B cell, or an aK562 cell). In the present methods, a nucleic acid described herein can be administered to the cell, for example, as nucleic acid without delivery vehicle, in combination with a delivery reagent. In some embodiments, any nucleic acid delivery method known in the art can be used in the methods described herein. Suitable delivery reagents include, but are not limited to, e.g., the Minis Transit TKO lipophilic reagent; lipofectin; lipofectamine; cellfectin; polycations (e.g., polylysine), atelocollagen, nanoplexes and liposomes. In some embodiments of the methods described herein, liposomes are used to deliver a nucleic acid to a cell or subject. Liposomes suitable for use in the methods described herein can be formed from standard vesicle-forming lipids, which generally include neutral or negatively charged phospholipids and a sterol, such as cholesterol. The selection of lipids is generally guided by consideration of factors such as the desired liposome size and half-life of the liposomes in the blood stream. A variety of methods are known for preparing liposomes, for example, as described in Szoka et al. (1980), Ann. Rev. Biophys. Bioeng. 9:467; and U.S. Pat. Nos. 4,235,871, 4,501,728, 4,837,028, and 5,019,369, the entire disclosures of which are herein incorporated by reference.

### Antibodies

In some aspects, the compositions and methods provided herein relate to antibodies, and antigen-binding fragments thereof, that bind specifically to a protein expressed on the plasma membrane of an EBV-infected or EBV-antigen presenting cell or a cancer cell (e.g., a protein comprising at least one of the epitopes listed in Table 1, or combinations thereof). In some embodiments, the antibodies bind to a particular epitope of one of the peptides provided herein, such as an EBV protein comprising an epitope with an amino acid sequence in Table 1, e.g., wherein the EBV protein is not a full-length EBV protein. In some embodiments, the epitope is an extracellular epitope. In some embodiments, the epitope is an epitope listed in Table 1. The antibodies can be polyclonal or monoclonal and can be, for example, murine, chimeric, humanized or fully human. The antibody may be a full-length immunoglobulin molecule, an scFv, a Fab fragment, an Fab′ fragment, a F(ab′)2 fragment, an Fv, a camelid antibody or a disulfide linked Fv. In some such embodiments, the antibodies contemplated herein are neutralizing antibodies,

Polyclonal antibodies can be prepared by immunizing a suitable subject (e.g., a mouse) with a peptide immunogen (e.g., at least one amino acid sequence listed in Table 1). In some embodiments, the peptide immunogen comprises an extracellular epitope of a target protein provided herein. The peptide antibody titer in the immunized subject can be monitored over time by standard techniques, such as with an enzyme linked immunosorbent assay (ELISA) using immobilized peptide. If desired, the antibody directed against the antigen can be isolated from the mammal (e.g., from the blood) and further purified by well-known techniques, such as protein A chromatography to obtain the IgG fraction.

At an appropriate time after immunization, e.g., when the antibody titers are highest, antibody-producing cells can be obtained from the subject and used to prepare monoclonal antibodies using standard techniques, such as the hybridoma technique originally described by Kohler and Milstein (1975) Nature 256:495-497) (see also Brown et al. (1981) J. Immunol. 127:539-46; Brown et al. (1980) J. Biol. Chem. 255:4980-83; Yeh et al. (1976) Proc. Natl. Acad. Sci. 76:2927-31; and Yeh et al. (1982) Int. J. Cancer 29:269-75), a human B cell hybridoma technique (Kozbor et al. (1983) Immunol. Today. 4:72), an EBV-hybridoma technique (Cole et al. (1985)Monoclonal Antibodies and Cancer Therapy. Alan R. Liss, Inc., pp. 77-96) or a trioma techniques. The technology for producing monoclonal antibody hybridomas is well known (see generally Kenneth, R. H. in Monoclonal Antibodies: A New Dimension In Biological Analyses. Plenum Publishing Corp., New York, New York (1980); Lerner, E. A. (1981) Yale J. Biol. Med. 54:387-402; Gefter, M. L. et al. (1977) Somatic Cell Genet. 3:231-36). Briefly, an immortal cell line (typically a myeloma) is fused to lymphocytes (typically splenocytes) from a mammal immunized with an immunogen as described above, and the culture supernatants of the resulting hybridoma cells are screened to identify a hybridoma producing a monoclonal antibody that binds to the peptide antigen, preferably specifically.

As an alternative to preparing monoclonal antibody-secreting hybridomas, a monoclonal antibody that binds to a target protein described herein can be obtained by screening a recombinant combinatorial immunoglobulin library with the appropriate peptide (e.g. a peptide comprising an epitope of Table 1) to thereby isolate immunoglobulin library members that bind the peptide.

Additionally, recombinant antibodies specific for a target protein provided herein and/or an extracellular epitope of a target protein provided herein, such as chimeric or humanized monoclonal antibodies, can be made using standard recombinant DNA techniques. Such chimeric and humanized monoclonal antibodies can be produced by recombinant DNA techniques known in the art, for example using methods described in U.S. Pat. Nos. 4,816,567; 5,565,332; Better et al. (1988) Science. 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. 84:214-218; Nishimura et al. (1987) Cancer Res. 47:999-1005; Wood et al. (1985) Nature 314:446-449; and Shaw et al. (1988) J. Natl. Cancer Inst. 80:1553-1559); Morrison, S. L. (1985) Science 229:1202-1207; Oi et al. (1986) Biotechniques. 4:214; Winter U.S. Pat. No. 5,225,539; Jones et al. (1986) Nature. 321:552-525; Verhoeyan et al. (1988) Science 239:1534; and Beidler et al. (1988) J. Immunol. 141:4053-4060.

Human monoclonal antibodies specific for a target protein provided herein and/or an extracellular epitope provided herein can be generated using transgenic or transchromosomal mice carrying parts of the human immune system rather than the mouse system. For example, “HuMAb mice” which contain a human immunoglobulin gene miniloci that encodes unrearranged human heavy (μ and γ) and κ light chain immunoglobulin sequences, together with targeted mutations that inactivate the endogenous μ and κ chain loci (Lonberg, N. et al. (1994) Nature. 368(6474): 856 859). Accordingly, the mice exhibit reduced expression of mouse IgM or κ, and in response to immunization, the introduced human heavy and light chain transgenes undergo class switching and somatic mutation to generate high affinity human IgGκ monoclonal antibodies (Lonberg, N. et al. (1994), supra; reviewed in Lonberg, N. (1994) Handbook of Experimental Pharmacology. 113:49 101; Lonberg, N. and Huszar, D. (1995) Intern. Rev. Immunol. Vol. 13: 65 93, and Harding, F. and Lonberg, N. (1995) Ann. N. Y Acad. Sci. 764:536 546). The preparation of HuMAb mice is described in Taylor, L. et al. (1992) Nucleic Acids Research. 20:6287 6295; Chen, J. et al. (1993) International Immunology. 5: 647 656; Tuaillon et al. (1993) Proc. Natl. Acad. Sci. USA 90:3720 3724; Choi et al. (1993) Nature Genetics. 4:117 123; Chen, J. et al. (1993) EMBO J. 12: 821 830; Tuaillon et al. (1994) J. Immunol. 152:2912 2920; Lonberg et al., (1994) Nature. 368(6474): 856 859; Lonberg, N. (1994) Handbook of Experimental Pharmacology. 113:49 101; Taylor, L. et al. (1994) International Immunology. 6: 579 591; Lonberg, N. and Huszar, D. (1995) Intern. Rev. Immunol. Vol. 13: 65 93; Harding, F. and Lonberg, N. (1995) Ann. N.Y. Acad. Sci. 764:536 546; Fishwild, D. et al. (1996) Nature Biotechnology 14: 845 851. See further, U.S. Pat. Nos. 5,545,806; 5,569,825; 5,625,126; 5,789,650; 5,877,397; 5,661,016; 5,814,318; 5,874,299; 5,770,429; and 5,545,807.

In some embodiments, the antibodies provided herein are able to bind to an epitope listed in Table 1 with a dissociation constant of no greater than 10⁻⁶, 10⁻⁷, 10⁻⁸ or 10⁻⁹ M. Standard assays to evaluate the binding ability of the antibodies are known in the art, including for example, ELISAs, Western blots and RIAs. The binding kinetics (e.g., binding affinity) of the antibodies also can be assessed by standard assays known in the art, such as by Biacore analysis.

In some embodiments the antibody is part of an antibody-drug conjugate. Antibody-drug conjugates are therapeutic molecules comprising an antibody (e.g., an antibody that binds to a protein listed in Table 1) linked to a biologically active agent, such as a cytotoxic agent or an antiviral agent. In some embodiments, the biologically active agent is linked to the antibody via a chemical linker. Such linkers can be based on any stable chemical motif, including disulfides, hydrazones, peptides or thioethers. In some embodiments, the linker is a cleavable linker and the biologically active agent is released from the antibody upon antibody binding to the plasma membrane target protein. In some embodiments, the linker is a noncleavable linker.

In some embodiments, the antibody-drug conjugate comprises an antibody linked to a cytotoxic agent. In some embodiments, any cytotoxic agent able to kill EBV-infected cells can be used. In some embodiments, the cytotoxic agent is MMAE, DM-1, a maytansinoid, a doxorubicin derivative, an auristatin, a calcheamicin, CC-1065, an aduocarmycin or an anthracycline.

In some embodiments, the antibody-drug conjugate comprises an antibody linked to an antiviral agent. In some embodiments, any antiviral agent capable of inhibiting EBV replication is used. In some embodiments, the antiviral agent is ganciclovir, valganciclovir, foscarnet, cidofovir, acyclovir, formivirsen, maribavir, BAY 38-4766 or GW275175X. In some embodiments, provided herein are vaccines comprising the antibodies or antibody-drug conjugates described herein.

### Cells

In some aspects, provided herein are antigen-presenting cells (APCs) that express on their surface an MHC that present one or more peptides comprising an EBV epitope described herein (e.g., APCs that present one or more of the EBV epitopes listed in Table 1). In some embodiments, the MHC is a class I MHC. In some embodiments, the MHC is a class II MHC. In some embodiments, the class I MHC has an α chain polypeptide that is HLA-A, HLA-B, HLA-C, HLA-E, HLA-F, HLA-g, HLA-K or HLA-L. In some embodiments, the class II MHC has an a chain polypeptide that is HLA-DMA, HLA-DOA, HLA-DPA, HLA-DQA or HLA-DRA. In some embodiments, the class II MHC has a β chain polypeptide that is HLA-DMB, HLA-DOB, HLA-DPB, HLA-DQB or HLA-DRB.

In some embodiments, the APCs are B cells, antigen-presenting T-cells, dendritic cells, or artificial antigen-presenting cells (e.g., aK562 cells). Dendritic cells for use in the process may be prepared by taking PBMCs from a patient sample and adhering them to plastic. Generally, the monocyte population sticks and all other cells can be washed off. The adherent population is then differentiated with IL-4 and GM-CSF to produce monocyte derived dendritic cells. These cells may be matured by the addition of IL-1β, IL-6, PGE-1 and TNF-α (which upregulates the important co-stimulatory molecules on the surface of the dendritic cell) and are then transduced with one or more of the peptides provided herein.

In some embodiments, the APC is an artificial antigen-presenting cell, such as an aK562 cell. In some embodiments, the artificial antigen-presenting cells are engineered to express CD80, CD83, 41BB-L, and/or CD86. Exemplary artificial antigen-presenting cells, including aK562 cells, are described U.S. Pat. Pub. No. 2003/0147869, which is hereby incorporated by reference.

In certain aspects, provided herein are methods of generating APCs that present the one or more of the EBV epitopes described herein comprising contacting an APC with a peptide or, pool of peptides, comprising at least one EBV epitope described herein and/or with a nucleic acid encoding at least on EBV epitope described herein. In some embodiments, the APCs are irradiated.

In certain aspects, provided herein are T cells (e.g., CD4 T cells and/or CD8 T cells) that express a TCR (e.g., an αβ TCR or a γδ TCR) that recognizes a peptide described herein (e.g., a peptide comprising at least one EBV epitope listed in Table 1) presented on a MHC. In some embodiments, the T cell is a CD8 T cell (a CTL) that expresses a TCR that recognizes a peptide described herein presented on a class I MHC. In some embodiments, the T cell is a CD4 T cell (a helper T cell) that recognizes a peptide described herein presented on a class II MHC.

In some aspects, provided herein are methods of generating, activating and/or inducing proliferation of T cells (e.g., CTLs) that recognize one or more of the EBV epitopes described herein. In some embodiments, a sample comprising CTLs (i.e., a PBMC sample) is incubated in culture with APCs provided herein (e.g., APCs that present a peptide comprising an EBV epitope described herein on a class I MHC complex). The APCs may be autologous to the subject from whom the T cells were obtained. In some embodiments, the sample containing T cells is incubated 2 or more times with APCs provided herein. In some embodiments, the T cells are incubated with the APCs in the presence of at least one cytokine, e.g., IL-4, IL-7 and/or IL-15. Exemplary methods for inducing proliferation of T cells using APCs are provided, for example, in U.S. Pat. Pub. No. 2015/0017723, which is hereby incorporated by reference. Alternatively, generating, activating and/or inducing proliferation of said T cells may comprise bringing a sample comprising CTLs (i.e., a PBMC sample) into contact with one or more peptides (e.g., a pool of peptides) comprising at least one of the CTL epitope amino acid sequences set forth in Table 1, or combinations thereof. In some embodiments, the sample comprising CTLs is brought into contact with a pool of peptides comprising each of the CTL epitope amino acid sequences set forth in SEQ ID NOs. 1-20.

In some aspects, provided herein are compositions (e.g., therapeutic compositions) comprising T cells and/or APCs provided herein. In some embodiments, such compositions are used to treat and/or prevent a cancer, an EBV infection, and/or an autoimmune disease in a subject by administering to the subject an effective amount of the composition. The T cells and/or APCs may be autologous or not autologous to the subject. In some embodiments, the T cells and/or APCs are stored in a cell bank before they are administered to the subject.

### Pharmaceutical Compositions

In some aspects, provided herein is a composition (e.g., a pharmaceutical composition, such as a vaccine composition), containing a polyepitope peptide or CTL described herein, or preparation thereof, formulated together with a pharmaceutically acceptable carrier, as well as methods of administering such pharmaceutical compositions.

Glycoproteins are critical to virus entry and can modify host cell behavior. The EBV genome encodes genes for 13 glycoproteins, 12 of which are expressed only during the productive, lytic replication cycle and one of which may be expressed during latency as well.

TABLE 2
EBV glycoproteins

| Protein | Gene | | | |
|---|---|---|---|---|
| name | name | Type | Expression | Function |
| gp350 | BLLF1 | Single pass type 1 | Late | Attachment |
| | | membrane | lytic/structural | |
| gB | BALF4 | Single pass type 1 | Late | Fusion |
| | | membrane | lytic/structural | |
| gH | BXLF2 | Single pass type 1 | Late | Regulation/triggering of |
| | | membrane | lytic/structural | fusion |
| gL | BKRF2 | Soluble associated | Late | Regulation/triggering of |
| | | with gH | lytic/structural | fusion |
| gp42 | BZLF2 | Single pass type 2 | Late | Triggering fusion/immune |
| | | membrane/soluble | lytic/structural | evasion |
| gM | BBRF3 | Multispanning | Late | Assembly and release |
| | | membrane | lytic/structural | |
| gN | BLRF2 | Single pass type 1 | Late | Assembly and release |
| | | membrane | lytic/structural | |
| BMRF2 | BMRF2 | Multispanning | Late | Epithelial cell attachment |
| | | membrane | lytic/structural | and spread |
| BDLF2 | BDLF2 | Single pass type 2 | Late | Epithelial spread |
| | | membrane | lytic/structural | |
| BDLF3 | BDLF3 | Single pass type 1 | Late | immune evasion |
| | | membrane | lytic/structural | |
| BILF2 | BILF2 | Single pass type 1 | Late | Unknown |
| | | membrane | lytic/structural | |
| BILF1 | BILF1 | Multispanning | Immediate | G-protein-coupled |
| | | membrane | early/early | receptor/immune evasion |
| BARF1 | BARF1 | Secreted | Latent and | CSF1 receptor/immune |
| | | | early lytic | evasion |

The most abundant of the EBV glycoproteins is gp350, the protein responsible for attachment of EBV to B lymphocytes. Following attachment to the B-cell surface, EBV enters the cell via fusion of its envelope with the cell membrane mediated by glycoproteins, gB, gHgL complex, and gp42. In addition, EBV glycoproteins are capable of manipulating the host cell. For example, BILF1 may downregulate expression of HLA class I molecules on the cell surface, targeting them for internalization and degradation in the lysosome; BARF1 may act as a soluble colony stimulating factor 1 (CSF-1) receptor that can block the differentiation of hematopoietic stem cells into macrophages or other related cell types; gp42 can interact with HLA class II/peptide complexes, impacting both virus entry and recognition by CD4⁺ T cells. Thus, the vaccine and/or pharmaceutical compositions disclosed herein may further comprise at least one viral glycoprotein selected from Table 2, or fragments thereof. In preferred embodiments, said vaccine and/or pharmaceutical compositions further comprise gp350, gB, gH, gL, gHgL complex, gp42, a fragment thereof, or any combination thereof. Most preferably, the vaccine and/or pharmaceutical compositions further comprise a combination of an EBV epitope-containing polyepitope protein and a gp350 polypeptide.

In other embodiments, the vaccine and/or pharmaceutical composition may further comprise an adjuvant. As used herein, the term “adjuvant” broadly refers to an immunological or pharmacological agent that modifies or enhances the immunological response to a composition in vitro or in vivo. For example, an adjuvant might increase the presence of an antigen over time, help absorb an antigen-presenting cell antigen, activate macrophages and lymphocytes and support the production of cytokines. By changing an immune response, an adjuvant might permit a smaller dose of the immune interacting agent or preparation to increase the dosage effectiveness or safety. For example, an adjuvant might prevent T cell exhaustion and thus increase the effectiveness or safety of a particular immune interacting agent or preparation. Examples of adjuvants include, but are not limited to, an immune modulatory protein, Adjuvant 65, α-GalCer, aluminum phosphate, aluminum hydroxide, calcium phosphate, β-Glucan Peptide, synthetic oligodeoxynucleotides (ODNs), CpG DNA, GPI-0100, lipid A and modified versions thereof (e.g., monophosphorylated lipid A, lipopolysaccharide, Lipovant, Montanide, N-acetyl-muramyl-L-alanyl-D-isoglutamine, Pam3CSK4, quil A and trehalose dimycolate). In preferred embodiments, the adjuvant comprises CpG DNA, such as synthetic oligodeoxynucleotides (ODNs) containing CpG motifs, preferably unmethylated CpG motifs. In some such embodiments, the adjuvant comprises amphiphilic CpG DNA. Without being bound by any particular theory, such CpG DNA-containing adjuvants may trigger cells that express Toll-like receptor 9 (including human plasmacytoid dendritic cells and B cells) to mount an innate immune response and improve the function of professional antigen-presenting cells and boost the generation of humoral and cell-mediated vaccine-specific immune responses. CpG ODNs are known in the art and can be identified based on structural characteristics and activity on human peripheral blood mononuclear cells (PBMCs), in particular B cells and plasmacytoid dendritic cells (pDCs). CpG ODNs known in the art that find use as adjuvant component(s) in the present EBV epitope-containing vaccine compositions described herein are described in, for example, Berry et al., Infection and Immunity 72(2):1019-1028 (2004), Maeyama et al., PLOS ONE 9(2):e88846 (2014), Cheng et al., Front Immunol. 2016; 7: 284 (2016), Vollmer et al., Advanced Drug Delivery Reviews 61(3):195-204 (2009), Ma et al., Science 365(6449):162-168 (2019), and Liu et al., Nature 507(7493):519-522 (2014), all of which are incorporated herein by reference.

Human B cell stimulation (e.g., cellular proliferation, CD80 and CD86 expression, immunoglobulin production and IL-6 secretion) may be achieved with ODNs that possess a nuclease-resistant phosphorothioate-modified backbone with one or more CpG motifs and no polyG motif. CpG ODNs that induce a Th-1 response, in addition to potent B cell stimulation, belong to the B class (also known as K type) and enhance the ability of dendritic cells to produce IL-12 and help polarize T cell responses in the TH1 direction. Activation of natural killer (NK) cells and human plasmacytoid dendritic cells to secrete interferon-α may be induced by CpG ODNs of the A class (also known as D type). C class CpG ODNs combine the properties of both A and B classes by being able to stimulate B cell and NK cell activation and IFN-α production.

Methods of preparing these formulations or compositions include the step of bringing into association an agent described herein with the carrier and, optionally, one or more accessory ingredients. In general, the formulations are prepared by uniformly and intimately bringing into association an agent described herein with liquid carriers, or finely divided solid carriers, or both, and then, if necessary, shaping the product. In some embodiments the components of the formulation may be modified so that the prophylactic and/or therapeutic immunotherapy is delivered to the lymph nodes to improve T cell activation. For example, and without limitation, an agent described herein may be conjugated, either directly or indirectly, with an albumin-binding carrier (e.g., a lipid moiety or lipophilic tail) thereby delivering the agent to lymph nodes which naturally accumulate serum albumin. Without being bound to any particular theory, the effectiveness of the compositions disclosed herein is improved with targeting to lymph nodes as they are abundant with dendritic cells (DCs), which present antigens to CD8⁺ T lymphocytes, initiating CTL responses. Accordingly, at least one of an immunogenic polypeptide as described herein, an EBV glycoprotein as described herein; an adjuvant as described herein; or any combination thereof, may comprise an albumin-binding moiety (e.g., an albumin-binding lipid or lipophilic tail). For example, immunogenic peptides (or pools thereof), as described herein, may be conjugated, directly or indirectly, to an albumin-binding lipid. In preferred embodiments, the adjuvant is conjugated to an albumin-binding lipid. Most preferably, the adjuvant is a CpG ODN conjugated with an albumin-binding lipid. Such “albumin hitchhiking” approaches are known in the art and examples of producing conjugated agents (e.g., vaccine components) can be found in Liu et al. (2014). “Structure-based Programming of Lymph Node Targeting in Molecular Vaccines.” Nature. 2014 Mar. 27; 507(7493): 519-522; Moynihan, et al. (2016). “Eradication of large established tumors in mice by combination immunotherapy that engages innate and adaptive immune responses.” Nat Med. 22(12): 1402-1410; Moynihan, et al. (2018). “Enhancement of Peptide Vaccine Immunogenicity by Increasing Lymphatic Drainage and Boosting Serum Stability.” Cancer Immunol Res. 6(9): 1025-1038; Ma, et al. (2019). “Enhanced CAR-T cell activity against solid tumors by vaccine boosting through the chimeric receptor.” Science. 365(6449): 162-168, incorporated herein by reference in their entirety. In certain embodiments, lipids conjugated to the CpG ODN adjuvant component can include, for example, cholesterol, or monoacyl or diacyl lipids.

In some aspects of the invention, provided herein are methods for generating a prophylactic or therapeutic treatment for herpesvirus infection (e.g., EBV infection) comprising combining an isolated immunogenic polypeptide, at least one herpesvirus glycoprotein, at least one adjuvant comprising a TLR agonist, and a pharmaceutically acceptable excipient, in a formulation suitable for administration to a subject; wherein the immunogenic polypeptide comprises at least one of the CTL epitope amino acid sequences set forth in Table 1. In preferred embodiments, the herpesvirus glycoprotein is derived from EBV and comprises at least one of gp350, gB, gH, gL, gHgL complex, gp42, any fragment thereof, or any combination thereof. Most preferably, the glycoprotein is EBV gp350. In some embodiments, the adjuvant comprises a TLR9 agonist. In some such embodiments, the adjuvant comprises a CpG ODN as described herein.

Pharmaceutical compositions of this invention suitable for parenteral administration comprise one or more agents described herein in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and non-aqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

Regardless of the route of administration selected, the agents of the present invention, which may be used in a suitable hydrated form, and/or the pharmaceutical compositions of the present invention, are formulated into pharmaceutically-acceptable dosage forms by conventional methods known to those of skill in the art.

### Therapeutic Methods

In some aspects, provided herein are methods for prophylactically or therapeutically treating a herpesvirus infection (e.g., an EBV infection) in a subject. Such methods may comprise administering to the subject a composition comprising an immunogenic polypeptide comprising amino acid sequences derived from each of a plurality of cytotoxic T-cell (CTL) epitopes, wherein the polypeptide comprises at least one of the CTL epitope amino acid sequences set forth in SEQ ID NOs. 1-20, or combinations thereof; at least one herpesvirus glycoprotein as disclosed herein; and an adjuvant as disclosed herein. In preferred embodiments, the immunogenic polypeptide comprises each of the CTL epitope amino acid sequences set forth in SEQ ID NOs. 1-20. Most preferably, the immunogenic polypeptide comprises the amino acid sequence set forth in SEQ ID NO. 21. In some such embodiments, each of the CTL epitopes are restricted by any one of the HLA class I specificities selected from HLA A*03, HLA A11, HLA A*0201, HLA A*1101, HLA A*2301, HLA A*3002, HLA B27, HLA B35.08/B35.01, HLA B*44:0, HLA B57*03, HLA B*0702, HLA B*0801, HLA B*1501, HLA B*3501, HLA B*3508, HLA B*4001, HLA B*4402, HLA B*4402, HLA B*4403, HLA B*4405, HLA B*5301, HLA B*5701, or HLA B*5801. Such CTL epitopes may be derived from any one of EBV antigens EBNA1, EBNA3A, EBNA3B, EBNA3C, LMP2, LMP2a, BMLF1, BZLF1, or BRLF1.

In certain embodiments, provided herein are methods of treating an EBV infection, cancer, and/or an autoimmune disease in a subject comprising administering to the subject a pharmaceutical composition provided herein.

In some embodiments, provided herein is a method of treating an EBV infection in a subject. In some embodiments, the subject treated is immunocompromised, or otherwise immunosuppressed. For example, in some embodiments, the subject has a T cell deficiency. The subject may have X-linked lymphoproliferative disease (XLP). In further embodiments, the subject may have, or be at risk of having benign reactive infection, such as infectious mononucleosis, oral hairy leukoplakia, and or chronic active EBV infection. In some embodiments, the subject has leukemia, lymphoma or multiple myeloma. In some embodiments, the subject is infected with HIV and/or has AIDS. In some embodiments, the subject has undergone a tissue, organ and/or bone marrow transplant. In some embodiments, the subject is being administered immunosuppressive drugs. In some embodiments, the subject has undergone and/or is undergoing a chemotherapy. In some embodiments, the subject has undergone and/or is undergoing B-cell depletion, such as by use of rituximab. In some embodiments, the subject has undergone and/or is undergoing radiation therapy.

In some embodiments, the subject is also administered an anti-viral drug that inhibits viral replication. For example, in some embodiments, the subject is administered ganciclovir, valganciclovir, foscarnet, cidofovir, acyclovir, formivirsen, maribavir, BAY 38-4766 or GW275175X.

Also provided herein are methods of treating an autoimmune disorder in a subject comprising administering to the subject a pharmaceutical composition provided herein. Such methods may be used to treat any autoimmune disease, preferably EBV-associated autoimmune diseases. Examples of autoimmune diseases include, for example, glomerular nephritis, arthritis, dilated cardiomyopathy-like disease, ulceous colitis, Sjogren syndrome, Crohn disease, systemic erythematodes, chronic rheumatoid arthritis, juvenile rheumatoid arthritis, Still's diease, multiple sclerosis, psoriasis, allergic contact dermatitis, polymyositis, pachyderma, periarteritis nodosa, rheumatic fever, vitiligo vulgaris, Behcet disease, Hashimoto disease, Addison disease, dermatomyositis, myasthenia gravis, Reiter syndrome, Graves' disease, anaemia perniciosa, sterility disease, pemphigus, autoimmune thrombopenic purpura, autoimmune hemolytic anemia, active chronic hepatitis, Addison's disease, anti-phospholipid syndrome, atopic allergy, autoimmune atrophic gastritis, achlorhydra autoimmune, celiac disease, Cushing's syndrome, dermatomyositis, discoid lupus erythematosus, Goodpasture's syndrome, Hashimoto's thyroiditis, idiopathic adrenal atrophy, idiopathic thrombocytopenia, insulin-dependent diabetes, Lambert-Eaton syndrome, lupoid hepatitis, lymphopenia, mixed connective tissue disease, pemphigoid, pemphigus vulgaris, pernicious anemia, phacogenic uveitis, polyarteritis nodosa, polyglandular autosyndromes, primary biliary cirrhosis, primary sclerosing cholangitis, Raynaud's syndrome, relapsing polychondritis, Schmidt's syndrome, limited scleroderma (or crest syndrome), sympathetic ophthalmia, systemic lupus erythematosis, Takayasu's arteritis, temporal arteritis, thyrotoxicosis, type b insulin resistance, type I diabetes, ulcerative colitis and Wegener's granulomatosis. In preferred embodiments, methods disclosed herein may be used to treat systemic lupus erythematosus (SLE), multiple sclerosis (MS), rheumatoid arthritis (RA), juvenile idiopathic arthritis (JIA), inflammatory bowel disease (IBD), celiac disease and type 1 diabetes.

Treatment of MS, includes treatment of all types and patterns of progression. Thus, preferred embodiments of the invention disclosed herein include treatment of relapsing-remitting MS (RRMS), secondary-progressive MS (SPMS), primary-progressive MS (PPMS), and/or progressive-relapsing MS (PRMS).

In some preferred embodiments, the methods provided herein are used to treat a systemic autoimmune disease (SAD). For example, in some such embodiments, the methods provided herein are used to treat rheumatoid arthritis, systemic lupus erythematosus and/or Sjögren's syndrome.

In further preferred embodiments, the methods provided herein are used to treat IBD. For example, the methods provided herein may be used to treat Crohn's disease (regional bowel disease, e.g., inactive and active forms), celiac disease (e.g., inactive or active forms) and/or ulcerative colitis (e.g., inactive and active forms). In some such embodiments, the methods provided herein may be used to treat irritable bowel syndrome, microscopic colitis, lymphocytic-plasmocytic enteritis, coeliac disease, collagenous colitis, lymphocytic colitis, eosinophilic enterocolitis, indeterminate colitis, infectious colitis (viral, bacterial or protozoan, e.g. amoebic colitis) (e.g., clostridium dificile colitis), pseudomembranous colitis (necrotizing colitis), ischemic inflammatory bowel disease, Behcet's disease, sarcoidosis, scleroderma, IBD-associated dysplasia, dysplasia associated masses or lesions, and/or primary sclerosing cholangitis.

In some embodiments, the subject has cancer. EBV is etiologically associated with pre-malignant lymphoproliferative diseases (LPDs) and human tumors, being responsible for up to 200,000 new cases of cancer arising worldwide each year. In some embodiments, the methods described herein may be used to treat any cancerous or pre-cancerous tumor associated with EBV. In some embodiments, the cancer expresses one or more of the EBV epitopes provided herein (e.g., the EBV epitopes listed in Table 1). In some embodiments, the cancer includes a solid tumor. The great majority of the human population are seropositive for EBV, as it can establish lifelong latency, can have intermittent reactivation after primary infection, and has limited clinical symptoms in the majority of infected individuals. Notably, EBV persists as a latent infection within the B cell system and several of its diseases are of B cell origin, e.g., B cell lymphoproliferative disorders (B-LPDs) of the immunocompromised, Hodgkin Lymphoma (HL), Burkitt Lymphoma (BL), Diffuse Large B cell Lymphoma (DLBCL), plasmablastic lymphoma (PBL), and primary effusion lymphoma (PEL). EBV is also linked to tumors arising in other cellular niches which can harbor latent infection, e.g., LPDs and malignant lymphomas of T or NK cells, nasopharyngeal carcinoma (NPC), gastric carcinoma of epithelial origin, and leiomyosarcoma. Thus, cancers that may be treated by the methods and compositions provided herein include, but are not limited to, cancer cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; bronchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometrioid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; mammary paget's disease; acinar cell carcinoma; adenosquamous carcinoma; adenocarcinoma w/squamous metaplasia; malignant thymoma; malignant ovarian stromal tumor; malignant thecoma; malignant granulosa cell tumor; and malignant roblastoma; sertoli cell carcinoma; malignant leydig cell tumor; malignant lipid cell tumor; malignant paraganglioma; malignant extra-mammary paraganglioma; pheochromocytoma; glomangiosarcoma; malignant melanoma; amelanotic melanoma; superficial spreading melanoma; malignant melanoma in giant pigmented nevus; epithelioid cell melanoma; malignant blue nevus; sarcoma; fibrosarcoma; malignant fibrous histiocytoma; myxosarcoma; liposarcoma; leiomyosarcoma; rhabdomyosarcoma; embryonal rhabdomyosarcoma; alveolar rhabdomyosarcoma; stromal sarcoma; malignant mixed tumor; mullerian mixed tumor; nephroblastoma; hepatoblastoma; carcinosarcoma; malignant mesenchymoma; malignant brenner tumor; malignant phyllodes tumor; synovial sarcoma; malignant mesothelioma; dysgerminoma; embryonal carcinoma; malignant teratoma; malignant struma ovarii; choriocarcinoma; malignant mesonephroma; hemangiosarcoma; malignant hemangioendothelioma; kaposi's sarcoma; malignant hemangiopericytoma; lymphangiosarcoma; osteosarcoma; juxtacortical osteosarcoma; chondrosarcoma; malignant chondroblastoma; mesenchymal chondrosarcoma; giant cell tumor of bone; ewing's sarcoma; malignant odontogenic tumor; ameloblastic odontosarcoma; malignant ameloblastoma; ameloblastic fibrosarcoma; malignant pinealoma; chordoma; malignant glioma; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; malignant meningioma; neurofibrosarcoma; malignant neurilemmoma; malignant granular cell tumor; malignant lymphoma; Hodgkin's disease; Hodgkin's lymphoma; paragranuloma; small lymphocytic malignant lymphoma; diffuse large cell malignant lymphoma; follicular malignant lymphoma; mycosis fungoides; non-Hodgkin's lymphomas and related neoplasms; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia. More preferably, such cancers that may be treated include undifferentiated carcinomas of nasopharyngeal type (UNCT); nasopharyngeal carcinoma (NPC), including non-keratinizing and keratinizing subtypes; gastric carcinoma, including UNCTs and adenocarcinomas; Burkitt lymphoma, including endemic, sporadic, and AIDS-associated subtypes; B-lymphoproliferative diseases (B-LPDs), such as post-transplant B-LPD and HIV-related B-LPD; Diffuse large B cell lymphomas (DLBCLs), such as HIV-related DLBCL, pyothorax-associated lymphoma (PAL), and DLBCL not otherwise specified; T and NK-cell lymphoproliferative diseases (T/NK LPDs), including chronic active Epstein-Barr virus infection (CAEBV), Extra-nodal T/NK lymphomas, and Aggressive NK lymphomas; nodular lymphocyte-predominant Hodgkin lymphoma (NLPHL); and classic Hodgkin's lymphomas (cHLs) of all subtypes, including nodular sclerosis cHL, mixed cellularity cHL, Lymphocyte depleted cHL, Lymphocyte rich cHL, and HIV-related cHL.

In some embodiments, the subject is also administered an anti-cancer compound. Exemplary anti-cancer compounds include, but are not limited to, Alemtuzumab (Campath®), Alitretinoin (Panretin®), Anastrozole (Arimidex®), Bevacizumab (Avastin®), Bexarotene (Targretin®), Bortezomib (Velcade®), Bosutinib (Bosulif®), Brentuximab vedotin (Adcetris®), Cabozantinib (Cometriq™), Carfilzomib (Kyprolis™), Cetuximab (Erbitux®), Crizotinib (Xalkori®), Dasatinib (Sprycel®), Denileukin diftitox (Ontak®), Erlotinib hydrochloride (Tarceva®), Everolimus (Afinitor®), Exemestane (Aromasin®), Fulvestrant (Faslodex®), Gefitinib (Iressa®), Ibritumomab tiuxetan (Zevalin®), Imatinib mesylate (Gleevec®), Ipilimumab (Yervoy™), Lapatinib ditosylate (Tykerb®), Letrozole (Femara®), Nilotinib (Tasigna®), Ofatumumab (Arzerra®), Panitumumab (Vectibix®), Pazopanib hydrochloride (Votrient®), Pertuzumab (Perjeta™), Pralatrexate (Folotyn®), Regorafenib (Stivarga®), Rituximab (Rituxan®), Romidepsin (Istodax®), Sorafenib tosylate (Nexavar®), Sunitinib malate (Sutent®), Tamoxifen, Temsirolimus (Torisel®), Toremifene (Fareston®), Tositumomab and 131I-tositumomab (Bexxar®), Trastuzumab (Herceptin®), Tretinoin (Vesanoid®), Vandetanib (Caprelsa®), Vemurafenib (Zelboraf®), Vorinostat (Zolinza®), and Ziv-aflibercept (Zaltrap®).

In some embodiments, the subject is also administered a chemotherapeutic agent. Examples of such chemotherapeutic agents include, but are not limited to, alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall; dynemicin, including dynemicin A; bisphosphonates, such as clodronate; an esperamicin; as well as neocarzinostatin chromophore and related chromoprotein enediyne antibiotic chromophores, aclacinomysins, actinomycin, authrarnycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and doxetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluoromethylornithine (DMFO); retinoids such as retinoic acid; capecitabine; and pharmaceutically acceptable salts, acids or derivatives of any of the above.

In some embodiments, the subject is also administered an immunotherapeutic agent. Immunotherapy refers to a treatment that uses a subject's immune system to treat cancer, e.g. cancer vaccines, cytokines, use of cancer-specific antibodies, T cell therapy, and dendritic cell therapy.

In some embodiments, the subject is also administered an immune modulatory protein. Examples of immune modulatory proteins include, but are not limited to, B lymphocyte chemoattractant (“BLC”), C-C motif chemokine 11 (“Eotaxin-1”), Eosinophil chemotactic protein 2 (“Eotaxin-2”), Granulocyte colony-stimulating factor (“G-CSF”), Granulocyte macrophage colony-stimulating factor (“GM-CSF”), 1-309, Intercellular Adhesion Molecule 1 (“ICAM-1”), Interferon gamma (“IFN-gamma”), Interlukin-1 alpha (“IL-1 alpha”), Interleukin-1 beta (“IL-1 beta”), Interleukin 1 receptor antagonist (“IL-1 ra”), Interleukin-2 (“IL-2”), Interleukin-4 (“IL-4”), Interleukin-5 (“IL-5”), Interleukin-6 (“IL-6”), Interleukin-6 soluble receptor (“IL-6 sR”), Interleukin-7 (“IL-7”), Interleukin-8 (“IL-8”), Interleukin-10 (“IL-10”), Interleukin-11 (“IL-11”), Subunit beta of Interleukin-12 (“IL-12 p40” or “IL-12 p′70”), Interleukin-13 (“IL-13”), Interleukin-15 (“IL-15”), Interleukin-16 (“IL-16”), Interleukin-17 (“IL-17”), Chemokine (C-C motif) Ligand 2 (“MCP-1”), Macrophage colony-stimulating factor (“M-CSF”), Monokine induced by gamma interferon (“MIG”), Chemokine (C-C motif) ligand 2 (“MIP-1 alpha”), Chemokine (C-C motif) ligand 4 (“MIP-1 beta”), Macrophage inflammatory protein-1-delta (“MIP-1 delta”), Platelet-derived growth factor subunit B (“PDGF-BB”), Chemokine (C-C motif) ligand 5, Regulated on Activation, Normal T cell Expressed and Secreted (“RANTES”), TIMP metallopeptidase inhibitor 1 (“TIMP-1”), TIMP metallopeptidase inhibitor 2 (“TIMP-2”), Tumor necrosis factor, lymphotoxin-alpha (“TNF alpha”), Tumor necrosis factor, lymphotoxin-beta (“TNF beta”), Soluble TNF receptor type 1 (“sTNFRI”), sTNFRIIAR, Brain-derived neurotrophic factor (“BDNF”), Basic fibroblast growth factor (“bFGF”), Bone morphogenetic protein 4 (“BMP-4”), Bone morphogenetic protein 5 (“BMP-5”), Bone morphogenetic protein 7 (“BMP-7”), Nerve growth factor (“b-NGF”), Epidermal growth factor (“EGF”), Epidermal growth factor receptor (“EGFR”), Endocrine-gland-derived vascular endothelial growth factor (“EG-VEGF”), Fibroblast growth factor 4 (“FGF-4”), Keratinocyte growth factor (“FGF-7”), Growth differentiation factor 15 (“GDF-15”), Glial cell-derived neurotrophic factor (“GDNF”), Growth Hormone, Heparin-binding EGF-like growth factor (“HB-EGF”), Hepatocyte growth factor (“HGF”), Insulin-like growth factor binding protein 1 (“IGFBP-1”), Insulin-like growth factor binding protein 2 (“IGFBP-2”), Insulin-like growth factor binding protein 3 (“IGFBP-3”), Insulin-like growth factor binding protein 4 (“IGFBP-4”), Insulin-like growth factor binding protein 6 (“IGFBP-6”), Insulin-like growth factor 1 (“IGF-1”), Insulin, Macrophage colony-stimulating factor (“M-CSF R”), Nerve growth factor receptor (“NGF R”), Neurotrophin-3 (“NT-3”), Neurotrophin-4 (“NT-4”), Osteoclastogenesis inhibitory factor (“Osteoprotegerin”), Platelet-derived growth factor receptors (“PDGF-AA”), Phosphatidylinositol-glycan biosynthesis (“PIGF”), Skp, Cullin, F-box containing complex (“SCF”), Stem cell factor receptor (“SCF R”), Transforming growth factor alpha (“TGFalpha”), Transforming growth factor beta-1 (“TGF beta 1”), Transforming growth factor beta-3 (“TGF beta 3”), Vascular endothelial growth factor (“VEGF”), Vascular endothelial growth factor receptor 2 (“VEGFR2”), Vascular endothelial growth factor receptor 3 (“VEGFR3”), VEGF-D 6Ckine, Tyrosine-protein kinase receptor UFO (“Axl”), Betacellulin (“BTC”), Mucosae-associated epithelial chemokine (“CCL28”), Chemokine (C-C motif) ligand 27 (“CTACK”), Chemokine (C-X-C motif) ligand 16 (“CXCL16”), C-X-C motif chemokine 5 (“ENA-78”), Chemokine (C-C motif) ligand 26 (“Eotaxin-3”), Granulocyte chemotactic protein 2 (“GCP-2”), GRO, Chemokine (C-C motif) ligand 14 (“HCC-1”), Chemokine (C-C motif) ligand 16 (“HCC-4”), Interleukin-9 (“IL-9”), Interleukin-17 F (“IL-17F”), Interleukin-18-binding protein (“IL-18 BPa”), Interleukin-28 A (“IL-28A”), Interleukin 29 (“IL-29”), Interleukin 31 (“IL-31”), C-X-C motif chemokine 10 (“IP-10”), Chemokine receptor CXCR3 (“I-TAC”), Leukemia inhibitory factor (“LIF”), Light, Chemokine (C motif) ligand (“Lymphotactin”), Monocyte chemoattractant protein 2 (“MCP-2”), Monocyte chemoattractant protein 3 (“MCP-3”), Monocyte chemoattractant protein 4 (“MCP-4”), Macrophage-derived chemokine (“MDC”), Macrophage migration inhibitory factor (“MIF”), Chemokine (C-C motif) ligand 20 (“MIP-3 alpha”), C-C motif chemokine 19 (“MIP-3 beta”), Chemokine (C-C motif) ligand 23 (“MPIF-1”), Macrophage stimulating protein alpha chain (“MSPalpha”), Nucleosome assembly protein 1-like 4 (“NAP-2”), Secreted phosphoprotein 1 (“Osteopontin”), Pulmonary and activation-regulated cytokine (“PARC”), Platelet factor 4 (“PF4”), Stroma cell-derived factor-1 alpha (“SDF-1 alpha”), Chemokine (C-C motif) ligand 17 (“TARC”), Thymus-expressed chemokine (“TECK”), Thymic stromal lymphopoietin (“TSLP 4-IBB”), CD 166 antigen (“ALCAM”), Cluster of Differentiation 80 (“B7-1”), Tumor necrosis factor receptor superfamily member 17 (“BCMA”), Cluster of Differentiation 14 (“CD14”), Cluster of Differentiation 30 (“CD30”), Cluster of Differentiation 40 (“CD40 Ligand”), Carcinoembryonic antigen-related cell adhesion molecule 1 (biliary glycoprotein) (“CEACAM-1”), Death Receptor 6 (“DR6”), Deoxythymidine kinase (“Dtk”), Type 1 membrane glycoprotein (“Endoglin”), Receptor tyrosine-protein kinase erbB-3 (“ErbB3”), Endothelial-leukocyte adhesion molecule 1 (“E-Selectin”), Apoptosis antigen 1 (“Fas”), Fms-like tyrosine kinase 3 (“Flt-3L”), Tumor necrosis factor receptor superfamily member 1 (“GITR”), Tumor necrosis factor receptor superfamily member 14 (“HVEM”), Intercellular adhesion molecule 3 (“ICAM-3”), IL-1 R4, IL-1 RI, IL-10 Rbeta, IL-17R, IL-2Rgamma, IL-21R, Lysosome membrane protein 2 (“LIMPII”), Neutrophil gelatinase-associated lipocalin (“Lipocalin-2”), CD62L (“L-Selectin”), Lymphatic endothelium (“LYVE-1”), MHC class I polypeptide-related sequence A (“MICA”), MHC class I polypeptide-related sequence B (“MICB”), NRGl-betal, Beta-type platelet-derived growth factor receptor (“PDGF Rbeta”), Platelet endothelial cell adhesion molecule (“PECAM-1”), RAGE, Hepatitis A virus cellular receptor 1 (“TIM-1”), Tumor necrosis factor receptor superfamily member IOC (“TRAIL R3”), Trappin protein transglutaminase binding domain (“Trappin-2”), Urokinase receptor (“uPAR”), Vascular cell adhesion protein 1 (“VCAM-1”), XEDAR, Activin A, Agouti-related protein (“AgRP”), Ribonuclease 5 (“Angiogenin”), Angiopoietin 1, Angiostatin, Cathepsin S, CD40, Cryptic family protein D3 (“Cripto-1”), DAN, Dickkopf-related protein 1 (“DKK-1”), E-Cadherin, Epithelial cell adhesion molecule (“EpCAM”), Fas Ligand (FasL or CD95L), Fcg RIIB/C, FoUistatin, Galectin-7, Intercellular adhesion molecule 2 (“ICAM-2”), IL-13 R1, IL-13R2, IL-17B, IL-2 Ra, IL-2 Rb, IL-23, LAP, Neuronal cell adhesion molecule (“NrCAM”), Plasminogen activator inhibitor-1 (“PAI-1”), Platelet derived growth factor receptors (“PDGF-AB”), Resistin, stromal cell-derived factor 1 (“SDF-1 beta”), sgp130, Secreted frizzled-related protein 2 (“ShhN”), Sialic acid-binding immunoglobulin-type lectins (“Siglec-5”), ST2, Transforming growth factor-beta 2 (“TGF beta 2”), Tie-2, Thrombopoietin (“TPO”), Tumor necrosis factor receptor superfamily member 10D (“TRAIL R4”), Triggering receptor expressed on myeloid cells 1 (“TREM-1”), Vascular endothelial growth factor C (“VEGF-C”), VEGFR1, Adiponectin, Adipsin (“AND”), Alpha-fetoprotein (“AFP”), Angiopoietin-like 4 (“ANGPTL4”), Beta-2-microglobulin (“B2M”), Basal cell adhesion molecule (“BCAM”), Carbohydrate antigen 125 (“CA125”), Cancer Antigen 15-3 (“CA15-3”), Carcinoembryonic antigen (“CEA”), cAMP receptor protein (“CRP”), Human Epidermal Growth Factor Receptor 2 (“ErbB2”), Follistatin, Follicle-stimulating hormone (“FSH”), Chemokine (C-X-C motif) ligand 1 (“GRO alpha”), human chorionic gonadotropin (“beta HCG”), Insulin-like growth factor 1 receptor (“IGF-1 sR”), IL-1 sRII, IL-3, IL-18 Rb, IL-21, Leptin, Matrix metalloproteinase-1 (“MMP-1”), Matrix metalloproteinase-2 (“MMP-2”), Matrix metalloproteinase-3 (“MMP-3”), Matrix metalloproteinase-8 (“MMP-8”), Matrix metalloproteinase-9 (“MMP-9”), Matrix metalloproteinase-10 (“MMP-10”), Matrix metalloproteinase-13 (“MMP-13”), Neural Cell Adhesion Molecule (“NCAM-1”), Entactin (“Nidogen-1”), Neuron specific enolase (“NSE”), Oncostatin M (“OSM”), Procalcitonin, Prolactin, Prostate specific antigen (“PSA”), Sialic acid-binding Ig-like lectin 9 (“Siglec-9”), ADAM 17 endopeptidase (“TACE”), Thyroglobulin, Metalloproteinase inhibitor 4 (“TIMP-4”), TSH2B4, Disintegrin and metalloproteinase domain-containing protein 9 (“ADAM-9”), Angiopoietin 2, Tumor necrosis factor ligand superfamily member 13/Acidic leucine-rich nuclear phosphoprotein 32 family member B (“APRIL”), Bone morphogenetic protein 2 (“BMP-2”), Bone morphogenetic protein 9 (“BMP-9”), Complement component 5a (“C5a”), Cathepsin L, CD200, CD97, Chemerin, Tumor necrosis factor receptor superfamily member 6B (“DcR3”), Fatty acid-binding protein 2 (“FABP2”), Fibroblast activation protein, alpha (“FAP”), Fibroblast growth factor 19 (“FGF-19”), Galectin-3, Hepatocyte growth factor receptor (“HGF R”), IFN-alpha/beta R2, Insulin-like growth factor 2 (“IGF-2”), Insulin-like growth factor 2 receptor (“IGF-2 R”), Interleukin-1 receptor 6 (“IL-1R6”), Interleukin 24 (“IL-24”), Interleukin 33 (“IL-33”, Kallikrein 14, Asparaginyl endopeptidase (“Legumain”), Oxidized low-density lipoprotein receptor 1 (“LOX-1”), Mannose-binding lectin (“MBL”), Neprilysin (“NEP”), Notch homolog 1, translocation-associated (Drosophila) (“Notch-1”), Nephroblastoma overexpressed (“NOV”), Osteoactivin, Programmed cell death protein 1 (“PD-1”), N-acetylmuramoyl-L-alanine amidase (“PGRP-5”), Serpin A4, Secreted frizzled related protein 3 (“sFRP-3”), Thrombomodulin, Toll-like receptor 2 (“TLR2”), Tumor necrosis factor receptor superfamily member 10A (“TRAIL R1”), Transferrin (“TRF”), WIF-1ACE-2, Albumin, AMICA, Angiopoietin 4, B-cell activating factor (“BAFF”), Carbohydrate antigen 19-9 (“CA19-9”), CD 163, Clusterin, CRT AM, Chemokine (C-X-C motif) ligand 14 (“CXCL14”), Cystatin C, Decorin (“DCN”), Dickkopf-related protein 3 (“Dkk-3”), Delta-like protein 1 (“DLL1”), Fetuin A, Heparin-binding growth factor 1 (“aFGF”), Folate receptor alpha (“FOLR1”), Furin, GPCR-associated sorting protein 1 (“GASP-1”), GPCR-associated sorting protein 2 (“GASP-2”), Granulocyte colony-stimulating factor receptor (“GCSF R”), Serine protease hepsin (“HAI-2”), Interleukin-17B Receptor (“IL-17B R”), Interleukin 27 (“IL-27”), Lymphocyte-activation gene 3 (“LAG-3”), Apolipoprotein A-V (“LDL R”), Pepsinogen I, Retinol binding protein 4 (“RBP4”), SOST, Heparan sulfate proteoglycan (“Syndecan-1”), Tumor necrosis factor receptor superfamily member 13B (“TACT”), Tissue factor pathway inhibitor (“TFPI”), TSP-1, Tumor necrosis factor receptor superfamily, member 10b (“TRAIL R2”), TRANCE, Troponin I, Urokinase Plasminogen Activator (“uPA”), Cadherin 5, type 2 or VE-cadherin (vascular endothelial) also known as CD144 (“VE-Cadherin”), WNT1-inducible-signaling pathway protein 1 (“WISP-1”), and Receptor Activator of Nuclear Factor κ B (“RANK”).

In some embodiments, the subject is also administered an immune checkpoint inhibitor. Immune checkpoint inhibition broadly refers to inhibiting the checkpoints that cancer cells can produce to prevent or downregulate an immune response. Examples of immune checkpoint proteins include, but are not limited to, CTLA4, PD-1, PD-L1, PD-L2, A2AR, B7-H3, B7-H4, BTLA, KIR, LAG3, TIM-3 or VISTA. Immune checkpoint inhibitors can be antibodies or antigen-binding fragments thereof that bind to and inhibit an immune checkpoint protein. Examples of immune checkpoint inhibitors include, but are not limited to, nivolumab, pembrolizumab, pidilizumab, AMP-224, AMP-514, STI-A1110, TSR-042, RG-7446, BMS-936559, MEDI-4736, MSB-0020718C, AUR-012 and STI-A1010.

In some embodiments, a composition provided herein (e.g., a vaccine composition provided herein) is administered prophylactically to prevent cancer and/or an EBV infection. In some embodiments, the vaccine is administered to inhibit tumor cell expansion. The vaccine may be administered prior to or after the detection of cancer cells or EBV infected cells in a patient. Inhibition of tumor cell expansion is understood to refer to preventing, stopping, slowing the growth, or killing of tumor cells. In some embodiments, after administration of a vaccine comprising peptides, nucleic acids, antibodies, or APCs described herein, a proinflammatory response is induced. The proinflammatory immune response comprises production of proinflammatory cytokines and/or chemokines, for example, interferon gamma (IFN-γ) and/or interleukin 2 (IL-2). Proinflammatory cytokines and chemokines are well known in the art.

Conjoint therapy includes sequential, simultaneous and separate, and/or co-administration of the active compounds in such a way that the therapeutic effects of the first agent administered have not entirely disappeared when the subsequent treatment is administered. In some embodiments, the second agent may be co-formulated with the first agent or be formulated in a separate pharmaceutical composition.

Actual dosage levels of the active ingredients in the pharmaceutical compositions provided herein may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular agent employed, the route of administration, the time of administration, the rate of excretion or metabolism of the particular compound being employed, the duration of the treatment, other drugs, compounds and/or materials used in combination with the particular compound employed, the age, sex, weight, condition, general health and prior medical history of the patient being treated, and like factors well known in the medical arts.

In some aspects, provided herein is a method of identifying a subject suitable for a therapy provided herein (methods of treating an EBV infection and/or a cancer in a subject comprising administering to the subject a pharmaceutical composition provided herein). In some embodiments, the method comprises isolating a sample from the subject (e.g., a blood sample, a tissue sample, a tumor sample) and detecting the presence of an EBV epitope listed in Table 1 in the sample, e.g., using an ELISA assay, a western blot assay, a FACS assay, a fluorescent microscopy assay, an Edman degradation assay and/or a mass spectrometry assay (e.g., protein sequencing). In some embodiments, the presence of the EBV epitope is detected by detecting a nucleic acid encoding the EBV epitope. In some embodiments, the nucleic acid encoding the EBV epitope is detected using a nucleic acid probe, a nucleic acid amplification assay and/or a sequencing assay.

Examples of nucleic acid amplification assays that can be used in the methods provided herein include, but are not limited to polymerase chain reaction (PCR), LATE-PCR, ligase chain reaction (LCR), strand displacement amplification (SDA), transcription mediated amplification (TMA), self-sustained sequence replication (3 SR), Qβ replicase based amplification, nucleic acid sequence-based amplification (NASBA), repair chain reaction (RCR), boomerang DNA amplification (BDA) and/or rolling circle amplification (RCA).

In some embodiments the product of the amplification reaction is detected as an indication of the presence and/or identity of the bacteria in the sample. In some embodiments, the amplification product is detected after completion of the amplification reaction (i.e., endpoint detection). Examples of end-point detection methods include gel-electrophoresis based methods, probe-binding based methods (e.g., molecular beacons, HPA probes, lights-on/lights-off probes) and double-stranded DNA binding fluorescent-dye based methods (e.g., ethidium bromide, SYBR-green). In some embodiments, the amplification product is detected as it is produced in the amplification reaction (i.e., real-time detection). Examples of real-time detection methods include probe-binding based methods (e.g., molecular beacons, TaqMan probes, scorpion probes, lights-on/lights-off probes) and double-stranded DNA binding fluorescent-dye based methods (e.g., ethidium bromide, SYBR-green). In some embodiments, the product of the amplification reaction is detected and/or identified by sequencing (e.g., through the use of a sequencing assay described herein).

In some embodiments, the detection of the nucleic acid sequence comprises contacting the nucleic acid sequence with a nucleic acid probe that hybridizes specifically to the nucleic acid sequence. In some embodiments, the probe is detectably labeled. In some embodiments, the probe is labeled (directly or indirectly) with a fluorescent moiety. Examples of fluorescent moieties useful in the methods provided herein include, but are not limited to Allophycocyanin, Fluorescein, Phycoerythrin, Peridinin-chlorophyll protein complex, Alexa Fluor 350, Alexa Fluor 405, Alexa Fluor 430, Alexa Fluor 488, Alexa Fluor 514, Alexa Fluor 532, Alexa Fluor 546, Alexa Fluor 555, Alexa Fluor 568, Alexa Fluor 594, Alexa Fluor 633, Alexa Fluor 635, Alexa Fluor 647, Alexa Fluor 660, Alexa Fluor 680, Alexa Fluor 700, Alexa Fluor 750, Alexa Fluor 790, GFP, RFP, YFP, EGFP, mPlum, mCherry, mOrange, mKO, EYFP, mCitrine, Venus, YPet, Emerald, Cerulean and CyPet. In some embodiments, the probe is a molecular beacon probe, a molecular torch probe, a TaqMan probes, a SDA probe, a scorpion probe, a HPA probe, or a lights on/lights off probe.

In some embodiments, the nucleic acid sequence is detected by sequencing (e.g., whole genome sequencing, transcriptome sequence and/or targeted gene sequencing). Examples of sequencing processes that can be used in the methods provided herein include, but are not limited to, chain termination sequencing, massively parallel signature sequencing, ion semiconductor sequencing, polony sequencing, illumina sequencing, sequencing by ligation, sequencing by synthesis, pyrosequencing, single-molecule real-time sequencing, SOLiD sequencing, DNA nanoball sequencing, heliscope single molecule sequencing, single molecule real time sequencing, 454 sequencing, nanopore sequencing, tunneling currents DNA sequencing or sequencing by hybridization.

In some embodiments, the methods provided herein further comprise treating the identified subject using a therapeutic method provided herein (e.g., by administering to the subject a pharmaceutical composition provided herein).

### EXAMPLES

### Example 1: Vaccine Strategy

To overcome safety issues and to improve protection, the exemplified EBV vaccine disclosed herein was developed using recombinant EBVpolyepitope (EBVpoly), gp350 proteins, and human compatible adjuvant(s) to induce EBV-specific CD4⁺ and CD8⁺ T cell and neutralizing antibody responses against multiple antigens of EBV expressed in both lytic and latent phases of infection.

The EBVpoly is an artificial polyepitope protein consisting of 20 contiguous, minimal CD8⁺ T cell epitopes derived from eight EBV antigens (EBNA1, EBNA3A, EBNA3B, EBNA3C, LMP2A, BRLF1, BMLF1 and BZLF1). These epitopes are selected from multiple antigens to provide broad coverage of the human MHC class I alleles. To enhance the immunogenicity of each epitope embedded in the EBVpoly, a proteasomal liberation amino acid sequence (K, R or AD) was added to the carboxy terminus of the each epitope. This EBVpoly approach allows simultaneous induction of cytotoxic CD8⁺ T cell responses against multiple antigens without the need to develop complex vaccines containing multiple recombinant antigens with oncogenic potential.

Also included in the vaccine formulation is a recombinant EBV gp350 protein to target CD4⁺, CD8⁺ T cells responses and neutralizing antibody responses. The EBV gp350-specific neutralizing antibodies provide first line of defense against virus infection and CD4+ and CD8+ T cell responses will aid the elimination of virus infected cells. As described herein, the inventors have identified that the combination of EBVpoly with a gp350 peptide generates a vaccine composition with surprising efficacy.

Although recombinant, protein-based, subunit vaccines have been considered as safe vaccine approaches, they are at times poorly immunogenic and require co-administration of one or more immunostimulatory agents (e.g., adjuvants). Only a limited number of immunostimulatory agents, such as aluminum hydroxide, MF59 and monophosphyryl lipid A (MPL) have been used in licensed human vaccines. These agents are strong inducers of a protective humoral immune response. However, complex pathogens like EBV require induction of both humoral and cell-mediated immune responses. To fulfill this requirement, there is a need for the new generation immunostimulatory agents. Recently, immunostimulatory oligonucleotides comprising unmethylated cytosine-phosphate-guanine (CpG) motifs, motifs have been used to induce both humoral and cell-mediated immune responses in a number of vaccine formulations. However, a major challenge in employing CpG oligodeoxynucleotides (ODNs) as an immunostimulatory agent is the lack of an efficient delivery system with which to target the CpG motif in vivo to the immune cells of lymphoid organs. Due to their low molecular weight and high solubility, CpG ODNs tend to flush through lymph nodes within hours and are exposed to innate immune cells only briefly, inducing suboptimal immune responses. To overcome these challenges, next generation immunostimulatory CpG ODNs were developed by conjugating CpG ODNs with albumin-binding lipids, rendering them amphiphilic, and able to efficiently target immunostimulatory agents and vaccine antigens to the lymph nodes in vivo, thereby inducing a robust immune response as noted in Moynihan, et al. (2016). “Eradication of large established tumors in mice by combination immunotherapy that engages innate and adaptive immune responses.” Nat Med. 22(12): 1402-1410; Moynihan, et al. (2018). “Enhancement of Peptide Vaccine Immunogenicity by Increasing Lymphatic Drainage and Boosting Serum Stability.” Cancer Immunol Res. 6(9): 1025-1038; Ma, et al. (2019). “Enhanced CAR-T cell activity against solid tumors by vaccine boosting through the chimeric receptor.” Science. 365(6449): 162-168, incorporated herein by reference in their entirety. Following systemic administration, the lipid conjugate binds to endogenous albumin, which prevents the conjugates from rapidly entering into the blood stream, directing them to lymphatic and draining lymph nodes instead, where they accumulate due to filtering of albumin by antigen presenting cells.

TABLE 3

| Sequence identifier | Biological sequence |
|---|---|
| EBVpoly20PL-NH | M H P V G E A D Y F E Y R S S C |
| amino acid sequence | S S C P L S K I A D R P P I F I R R L K |
| (SEQ ID No. 21) | F L R G R A Y G L R G L C T L V A M L A |
| | D E E C D S E L E I K R Y K C L G G L L |
| | T M V A D R A K F K Q L L R A T I G T A |
| | M Y K A D T Y G P V F M C L K L P E P L |
| | P Q G Q L T A Y K I E D P P F N S L A D |
| | V S F I E F V G W K E E N L L D F V R F |
| | M G V K Q N G A L A I N T F R P Y L F W |
| | L A A I R A Y S S W M Y S Y A D R V R A |
| | Y T Y S K A D R R I Y D L I E L R V E I |
| | T P Y K P T W A D - |
| EBVpoly20PL-NH | atgcatccagttggtgaagcagactactttgaataccgttcctcttgc |
| nucleotide sequence | agctcgtgtccgctgagcaagattgcagatcgtccgccgatcttcatccgtcgtttgaaa |
| (SEQ ID No. 42) | tttctgcgcggtcgcgcgtacggcttgcgtggtctgtgcaccctggtggccatgctggcg |
| | gacgaggagtgtgatagcgagctcgaaatcaaacgctataagtgcctgggtggccttctg |
| | acgatggttgctgaccgtgcgaagtttaagcaactgctgcgcgccaccattggtacggca |
| | atgtataaagctgacacctatggcccggttttcatgtgtctgaagctgccggagccgctg |
| | ccgcagggtcaactgaccgcatacaagattgaggacccgccgttcaatagcctggcggac |
| | gtgagcttcattgaatttgtcggctggaaagaagagaatttgctggacttcgtccgcttc |
| | atgggcgtgaaacagaacggtgctctggcaatcaacacgtttcgtccgtacctgttctgg |
| | ctggcggccattcgtgcgtatagcagctggatgtacagctatgccgatcgtgtccgcgcg |
| | tacacctactccaaagcggatcgtcgtatctacgatctgatcgagctgcgtgttgaaatt |
| | accccgtataaacctacttgggcggattaa |
| gp350 amino acid | M E A A L L V C Q Y T I Q S L I H L |
| sequence (SEQ ID | T G E D P G F F N V E I P E F P F Y P |
| NO. 43) | T C N V C T A D V N V T I N F D V G G |
| | N V T T G E E Q Q V S L E S V D V Y F |
| | Q D V F G T M W C H H A E M Q N P V |
| | Y L I P E T V P Y I K W D N C N S T N |
| | I T A V V R A Q G L D V T L P L S L P |
| | T S A Q D S N F S V K T E M L G N E I |
| | D I E C I M E D G E I S Q V L P G D N |
| | K F N I T C S G Y E S H V P S G G I L |
| | T S T S P V A T P I P G T G Y A Y S L |
| | R L T P R P V S R F L G N N S I L Y V |
| | F Y S G N G P K A S G G D Y C I Q S N |
| | I V F S D E I P A S Q D M P T N T T D I |
| | T Y V G D N A T Y S V P M V T S E D A |
| | N S P N V T V T A F W A W P N N T E T |
| | D F K C K W T L T S G T P S G C E N I |
| | S G A F A S N R T F D I T V S G L G T |
| | A P K T L I I T R T A T N A T T T T H |
| | K V I F S K A P E S T T T S P T L N T T |
| | G F A D P N T T T G L P S S T H V P T |
| | N L T A P A S T G P T V S T A D V T S |
| | P T P A G T T S G A S P V T P S P S P |
| | W D N G T E S K A P D M T S S T S P V |
| | T T P T P N A T S P T P A V T T P T P |
| | N A T S P T P A V T T P T P N A T S P |
| | T L G K T S P T S A V T T P T P N A T |
| | K K H Q L D L D F G Q L T P H T K A V |
| | S P T L G K T S P T S A V T T P T P N |
| | Y Q P R G A F G G S E N A T N L F L L |
| | A T S P T L G K T S P T S A V T T P T |
| | P N A T G P T V G E T S P Q A N A T N |
| | E L L G A G E L A L T M R S K K L P I |
| | H T L G G T S P T P V V T S Q P K N A |
| | T S A V T T G Q H N I T S S S T S S M |
| | S L R P S S N P E T L S P S T S D N S T |
| | S H M P L L T S A H P T G G E N I T Q |
| | V T P A S I S T H H V S T S S P A P R P |
| | G T T S Q A S G P G N S S T S T K P G |
| | E V N V T K G T P P Q N A T S P Q A P |
| | S G Q K T A V P T V T S T G G K A N S |
| | T T G G K H T T G H G A R T S T E P T |
| | T D Y G G D S T T P R P R Y N A T T Y |
| | L P P S T S S K L R P R W T F T S P P |
| | V T T A Q A T V P V P P T S Q P R F S |
| | N L S D C A F R R N L S T S H T Y T T |
| | P P Y D D A E T Y V - |
| Gp350 nucleotide | atg gag gca gcc ttg ctt gtg tgt cag tac acc atc cag agc ctg atc cat |
| sequence (SEQ ID | ctc acg ggt gaa gat cct ggt ttt ttc aat gtt gag att ccg gaa ttc cca ttt |
| NO. 44) | tac ccc aca tgc aat gtt tgc acg gca gat gtc aat gta act atc aat ttc gat |
| | gtc ggg ggc aaa aag cat caa ctt gat ctt gac ttt ggc cag ctg aca ccc |
| | cat acg aag gct gtc tac caa cct cga ggt gca ttt ggt ggc tca gaa aat |
| | gcc acc aat ctc ttt cta ctg gag ctc ctt ggt gca gga gaa ttg gct cta act |
| | atg cgg tct aag aag ctt cca att aac gtc acc acc gga gag gag caa caa |
| | gta agc ctg gaa tct gta gat gtc tac ttt caa gat gtg ttt gga acc atg tgg |
| | tgc cac cat gca gaa atg caa aac ccc gtg tac ctg ata cca gaa aca gtg |
| | cca tac ata aag tgg gat aac tgt aat tct acc aat ata acg gca gta gtg |
| | agg gca cag ggg ctg gat gtc acg cta ccc tta agt ttg cca acg tca gct |
| | caa gac tcg aat ttc agc gta aaa aca gaa atg ctc ggt aat gag ata gat |
| | att gag tgt att atg gag gat ggc gaa att tca caa gtt ctg ccc gga gac |
| | aac aaa ttt aac atc acc tgc agt gga tac gag agc cat gtt ccc agc ggc |
| | gga att ctc aca tca acg agt ccc gtg gcc acc cca ata cct ggt aca |
| | ggg tat gca tac agc ctg cgt ctg aca cca cgt cca gtg tca cga ttt ctt |
| | ggc aat aac agt atc ctg tac gtg ttt tac tct ggg aat gga ccg aag gcg |
| | agc ggg gga gat tac tgc att cag tcc aac att gtg ttc tct gat gag att cca |
| | gct tca cag gac atg ccg aca aac acc aca gac atc aca tat gtg ggt gac |
| | aat gct acc tat tca gtg cca atg gtc act tct gag gac gca aac tcg cca |
| | aat gtt aca gtg act gcc ttt tgg gcc tgg cca aac aac act gaa act gac ttt |
| | aag tgc aaa tgg act ctc acc tcg ggg aca cct tcg ggt tgt gaa aat att tct |
| | ggt gca ttt gcg agc aat cgg aca ttt gac att act gtc tcg ggt ctt ggc acg |
| | gcc ccc aag aca ctc att atc aca cga acg gct acc aat gcc acc aca aca |
| | acc cac aag gtt ata ttc tcc aag gca ccc gag agc acc acc acc tcc cct |
| | acc ttg aat aca act gga ttt gct gat ccc aat aca acg aca ggt cta ccc |
| | agc tct act cac gtg cct acc aac ctc acc gca cct gca agc aca ggc ccc |
| | act gta tcc acc gcg gat gtc acc agc cca aca cca gcc ggc aca acg tca |
| | ggc gca tca ccg gtg aca cca agt cca tct cca tgg gac aac ggc aca gaa |
| | agt aag gcc ccc gac atg acc agc tcc acc tca cca gtg act acc cca acc |
| | cca aat gcc acc agc ccc acc cca gca gtg act acc cca acc cca aat gcc |
| | acc agc ccc acc cca gca gtg act acc cca acc cca aat gcc acc agc ccc |
| | acc ttg gga aaa aca agt cct acc tca gca gtg act acc cca acc cca aat |
| | gcc acc agc ccc acc ttg gga aaa aca agc ccc acc tca gca gtg act acc |
| | cca acc cca aat gcc acc agc ccc acc ttg gga aaa aca agc ccc acc tca |
| | gca gtg act acc cca acc cca aat gcc acc ggc cct act gtg gga gaa aca |
| | agt cca cag gca aat gcc acc aac cac acc tta gga gga aca agt ccc acc |
| | cca gta gtt acc agc caa cca aaa aat gca acc agt gct gtt acc aca ggc |
| | caa cat aac ata act tca agt tca acc tct tcc atg tca ctg aga ccc agt tca |
| | aac cca gag aca ctc agc ccc tcc acc agt gac aat tca acg tca cat atg |
| | cct tta cta acc tcc gct cac cca aca ggt ggt gaa aat ata aca cag gtg |
| | aca cca gcc tct atc agc aca cat cat gtg tcc acc agt tgg cca gca ccc |
| | cgc cca ggc acc acc agc caa gcg tca ggc cct gga aac agt tcc aca |
| | tcc aca aaa ccg ggg gag gtt aat gtc acc aaa ggc acg ccc ccc caa aat |
| | gca acg tgg ccc cag gcc ccc agt ggc caa aag acg gcg gtt ccc acg |
| | gtc acc tca aca ggt gga aag gcc aat tct acc acc ggt gga aag cac acc |
| | aca gga cat gga gcc cgg aca agt aca gag ccc acc aca gat tac ggc |
| | ggt gat tca act acg cca aga ccg aga tac aat gcg acc acc tat cta cct |
| | ccc agc act tct agc aaa ctg cgg ccc cgc tgg act ttt acg agc cca ccg |
| | gtt acc aca gcc caa gcc acc gtg cca gtc ccg cca acg tcc cag ccc aga |
| | ttc tca aac ctc tcc gac tgc gcc ttt agg cgt aac ttg tct aca tcc cat acc |
| | tac acc acc cca cca tat gat gac gcc gag acc tat gta taa |
| Soluble CpG7909 | 5′-tcgtcgttttgtcgttttgtcgtt-3′ |
| (SEQ ID NO. 45) | |

### Example 2: EBV Polyepitope Protein Construct Design, Protein Expression, Purification Process Development, and In Vitro Immunogenicity Evaluation

The EBVpoly protein sequence was designed in such a way that the carboxyl terminus of each epitope was joined by a proteasome liberation amino acid sequence (AD or K or R). (See Table 1.) Proteasome liberation amino acid sequences improves the immunogenicity of CD8⁺ T cell epitopes by enhancing proteasomal processing of the polyepitope protein by the antigen presenting cells (Dasari, et al. (2014). “Induction of innate immune signatures following polyepitope protein-glycoprotein B-TLR4&9 agonist immunization generates multifunctional CMV-specific cellular and humoral immunity.” Hum Vaccin Immunother. April; 10(4): 1064-1077). To achieve high level of EBVpoly protein expression, the amino acid sequence of the EBVpoly construct was translated into DNA sequence using optimised E. coli codons (SEQ ID NO. 42) and EBVpoly protein-encoding DNA sequence was synthetically constructed and cloned into an isopropyl-β-D-thiogalactopyraniside (IPTG) inducible plasmid, pJexpress 404 (Atum Bio, CA, United States). The synthetically designed EBVpoly construct was transformed into chemically competent E. coli DH5 a cells and the inducible expression plasmid was subsequently isolated and purified.

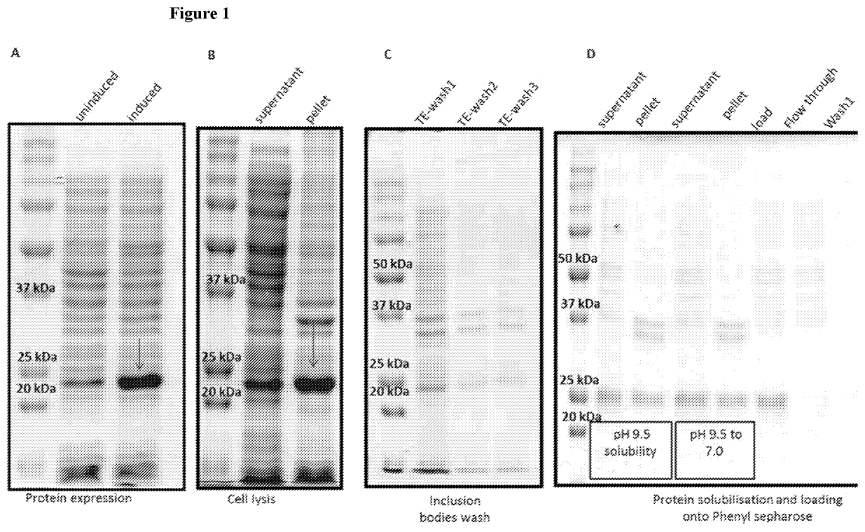

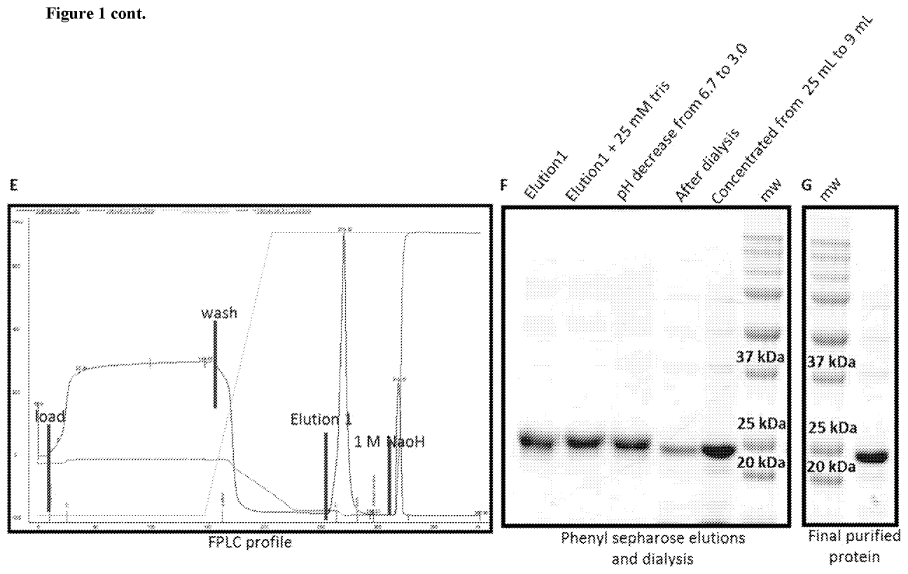

Chemically competent BL21-codonPlus (DE3) RP E. coli cells (Agilent Technologies, CA, United States) were transformed with the inducible EBVpoly expression vector. Transformed cells were plated on Luria Bertani (LB) agar supplemented with ampicillin (LB-Amp 10011 g/mL) and plates were incubated overnight at 37° C. An isolated colony was picked and inoculated into 10 ml of Terrific broth containing 100 μg/mL ampicillin (TB-Amp broth) and grown in a shaker at 37° C. and 200 rpm overnight. A small amount of the overnight grown culture was inoculated into 50 mL of TB-Amp broth and grown for 12 hours. About 1% of the 50 mL culture was transferred into 3 liters of TB-Amp broth and incubated until growth, as measured by optical density, reached to 0.6 at 600 nm. EBVpoly protein expression was induced by adding 1 mM/mL of IPTG to the culture and incubating for 5 hours at 25° C. At the end of the induction phase, the culture was harvested by centrifugation at 13,000 rpm for 15 minutes, and the cell pellet was re-suspended in 100 mL of lysis buffer (25 mM Tris pH 7.5, 5 mM EDTA, 0.5% TritonX 100, 0.5 mg/mL lysozyme) supplemented with a protease inhibitor cocktail (Roche, Mannheim, Germany) and incubated on ice for 30 minutes, followed by cell lysis by sonication. The sonication was carried out on ice for six 8-minute cycles (1 second on and off) with 10-minute breaks between each cycle. The lysate was centrifuged at 13,000 rpm for 30 minutes and supernatant and pellet fractions were analyzed using sodium dodecyl sulfate-polyacrylamide gel electrophoresis (SDS-PAGE) and comparing un-induced and induced samples. The EBVpoly expression vector produced high levels of EBVpoly protein (See FIG. 1A) However, due to the high hydrophobic nature of the linear CD8⁺ T cell epitopes, the induced EBVpoly protein was aggregated in the form of inclusion bodies (IBs) when the supernatant and pellet fractions from cell lysate were compared. (See FIG. 1B.) Approximately 2 grams of pellet (wet weight) was obtained for solubilization from every 3 L of induced culture. All IB washing, solubilization, and purification stages were carried out in the cold room. To eliminate host cell proteins and DNA contamination, IBs were washed three times with TE buffer (25 mM Tris and 5 mM EDTA pH 7.5). To make homogenous suspension, IBs were suspended in TE buffer, sonicated for 10 minutes (1-second on and off cycles) and then solution was incubated at 4° C., with stirring, for 30 minutes. At the end of every wash, solution was centrifuged at 13,000 rpm for 30 minutes. The supernatant obtained from all washes was analysed on SDS-PAGE gel to assess EBV protein loss. (See FIG. 1C.) IBs were then solubilized in 100 mM NaH₂PO₄, 10 mM Tris, 5 mM DTT, 8M urea, pH 9.5 buffer, under stirring for overnight at 4° C. The soluble protein was clarified by centrifugation at 13,000 rpm for 30 minutes and the pH of the solubilized protein was then decreased to pH 7.0.

To purify the solubilized EBVpoly protein, 20 mL of phenyl sepharose matrix (GE healthcare) was used. Prior to protein loading, the phenyl sepharose column was washed with 1 M NaOH, column pH was neutralized with distilled water, and then equilibrated with solubilization buffer (10 mM Tris, 50 mM NaH2Po4, 5 mM DTT, 0.5 M NaCl, 8 M urea pH 7.0). 150 mL of sample was loaded onto the column and the column was washed with buffer A to buffer B (0 to 100%) in 10 column volumes (CVs). (See FIG. 1D)
Buffer A (10 mM Tris, 50 mM NaH2Po4, 0.5 M NaCl, 8 M urea pH 7.0);
Buffer B (10 mM Tris, 50 mM NaH2Po4, 8 M urea pH 7.0).

After reaching buffer B concentration to 100%, EBVpoly protein bound to the column was eluted with a buffer containing 7.5 mM NaOH and 8M urea (3CVs). EBVpoly protein-positive elutions were collected in a total of 22 mL, and then buffered with 1 M tris pH7.5 to get a final concentration of 25 mM tris, pH 7.5. (See FIG. 1E.) EBVpoly protein pH was decreased from 7.6 to 3.0 using HCl. The purified EBVpoly protein was dialyzed against 25 mM glycine buffer, pH 3.0. After dialysis protein was concentrated from 25 mL to 9 mL and then passed through MustangE membrane (PALL Corporation, NY, USA) to eliminate endotoxin contaminants. All the samples were analysed on the 12% SDS-PAGE gel to show that EBVpoly protein was successfully expressed and purified to homogeneity using a bacterial expression system. (See FIGS. 1, F and G.)

### Example 3: Evaluation of EBVpoly Immunogenicity In Vitro

To determine the immunogenicity of EBV polyepitope protein, approximately 6×10⁶ PBMC from six different HLA-mapped, EBV-seropositive, healthy donors were stimulated with μg of EBVpoly protein for 1 h at 37° C. Following stimulation cells were washed with RPMI supplemented with 10% FCS and returned to incubation. Cells were cultured for 14 days to allow for T cell expansion; cultures were supplemented with medium containing RPMI and human recombinant IL2 on days 2, 5, 8 and 11.

Following said in vitro expansion of EBV-specific CD8⁺ T cells from healthy seropositive donors, cells were stimulated with 0.2 μg/mL of HLA matching peptides in the presence of human CD107a antibody conjugated to FITC, Golgiplug™ and Golgistop™ (BD Biosciences; CA, United States) for 4 hours at 37° C. and 6.5% CO₂. Cells were washed twice, then incubated with Live/Dead™ near IR, Pacific Blue™-conjugated anti-CD4 and PerCPCy5.5-conjugated anti-CD8. Cells were fixed and permeabilized using a BD Cytofix/Cytoperm™ kit (BD Biosciences; CA, United States). Then cells were incubated with PE-conjugated anti IL-2, APC-conjugated anti TNF and AF700-conjugated anti IFN-γ to determine intracellular cytokines secretion. Cells were acquired on a BD FACSCanto™ II and data was analysed using FlowJo™ software (Becton, Dickinson and Company, OR, Untied States). Thus, following stimulation of EBV-seropositive donor PBMC with EBVpoly protein, the expansion of EBV-specific CD8⁺ T cells could be assessed, as well as the ability of said expanded EBV-specific CD8⁺ T cells to express a degranulation marker (CD107a) and to secrete multiple cytokines (i.e., INFγ, TNF and IL2) by ICS.

Results

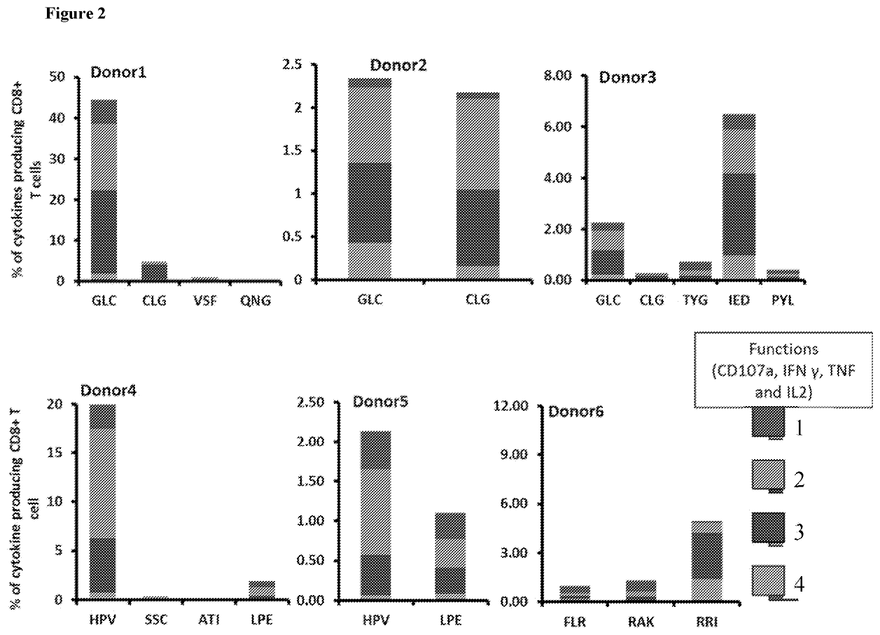

The data obtained from this experiment shows that EBVpoly protein was able to induce expansion of EBV-specific CD8⁺ T cells, restricted to multiple epitopes included in the EBVpoly protein, from all six donors. A large proportion of expanded cells demonstrated their functionality to degranulate (CD107a) and secrete multiple cytokines (INFγ, TNF and IL2). (See FIG. 2).

### Example 4: Schematic Representation of Experimental Design for Immunogenicity Evaluation of EBV Vaccine Formulated with amphCpG7909 or CpG7909 in Human HLA B35, A2, A24 and B8 Transgenic Mice

A number of studies on host immune responses against EBV have shown that both B and T cell immune responses play a fundamental role in the protection against EBV infection and control of EBV-associated diseases. Therefore, a vaccine formulation capable of inducting both humoral and cell-mediated immune responses may provide better protection against EBV-associated complications. In order to generate robust humoral and cell-mediated immune responses against EBV, the vaccine formulations were prepared by mixing EBV gp350 (10 μg) and EBVpoly protein (40 μg), with amphiphile-CpG7909 (1.2 nmol) or soluble CpG7909 (1.2 nmol) per dose in 100 μl volume. Adjuvant-alone control formulations were prepared by mixing lipid-conjugated CpG7909 (amphCpG7909) (1.2 nmol) or soluble CpG7909 (1.2 nmol) per dose in 100 μL volume.

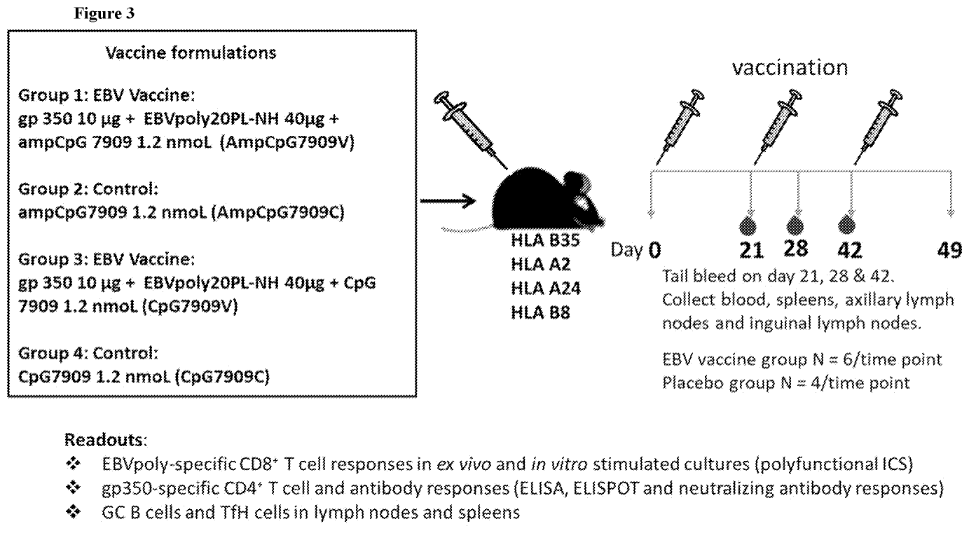

Human HLA B35, A2, A24 and B8 transgenic mice are deficient in expressing mouse MHC class I molecule and contain transgenes of the commonly expressed human HLA class I molecules. In order to evaluate the immunogenic response to EBV vaccine, two groups of mice for each HLA transgene were immunized with 3 doses comprising 40 μg of EBVpoly and 10 of gp350 proteins, formulated with either 1.2 nmol amphCpG7909 or 1.2 nmol CpG7909. Another two groups of mice were injected with 3 doses of 1.2 nmol AmpCpG7909 or 1.2 nmol CpG7909 to serve as placebo (adjuvant-alone control) group. All injections (vaccine group n=6 and control group n=4) were administered subcutaneously, 50 μl at each side of the tail base (10011.1 total) on day 0; boosted on day 21 and 42 with an identical vaccine or control formulation. The mice were tail bled on day 21, 28 and 42, and were finally sacrificed on day 49; blood, spleen, inguinal lymph nodes and axillary lymph nodes were collected to assess EBV-specific humoral and cell-mediated (e.g., T cell) responses using ICS assays, gp350 ELISpot, ELISA, and neutralizing antibody assays. (See FIG. 3).

### Example 5: Intracellular Cytokine Staining to Assess EBVpoly-Specific CD8⁺ T Cells Producing Multiple Cytokines

As described herein (see also schematic of FIG. 3), immunized Human HLA B35, A2, A24 and B8 transgenic mice were sacrificed on day 49 and single-cell suspensions were made from splenocytes. These cells were stimulated with either 0.2 μg/mL of HLA B35 (i.e., SEQ ID NO. 1 “HPV” and SEQ ID NO. 11 “LPEP”), HLA A2 (SEQ ID NO. 5 “GLC” and SEQ ID NO. 7 “CLG”), HLA A24 (SEQ ID NO. 10 “TYG” and SEQ ID NO. 16 “PYL”) and HLA B8 (SEQ ID NO. 4 “FLR” and SEQ ID NO. 8 “RAK”) restricted peptides to determine the EBV-specific CD8⁺ T cell responses for four hours in vitro, in the presence of Golgiplug™ and Golgistop™ for hours. Cells were washed twice, then incubated with, Live/Dead™ near IR, FITC-conjugated anti-CD4 and PerCP5.5 conjugated anti-CD8. Cells were fixed and permeabilized using a BD Cytofix/Cytoperm™ kit, then incubated with PE-conjugated anti-IFN-γ, PE-Cy7 conjugated anti-TNF, and APC conjugated anti-IL2 PE. Cells were acquired on a BD FACSCanto™ II and data was analyzed using FlowJo™ software.

To evaluate memory CD8⁺ T cell response induced following immunization with EBV vaccine formulated with amphCpG7909 or CpG7909; HLA B35, A2, A24 and B8 splenocytes were harvested, cultures were prepared (7×10⁶ splenocytes) and stimulated in vitro with 0.2 μg/mL of HLA B35 (i.e., SEQ ID NO. 1 “HPV” and SEQ ID NO. 11 “LPEP”), HLA A2 (SEQ ID NO. 5 “GLC” and SEQ ID NO. 7 “CLG”), HLA A24 (SEQ ID NO. 10 “TYG” and SEQ ID NO. 16 “PYL”) and HLA B8 (SEQ ID NO. 4 “FLR” and SEQ ID NO. 8 “RAK”) restricted peptides. To further expand memory EBVpoly-specific CD8⁺ T cells, cells were cultured in a 24 well plate for 10 days at 37° C., 10% CO₂, and were supplemented with IL-2 on days 2, 5 and 8. On day 10, the expanded T cells were stimulated with epitope peptides HLA B35 (i.e., SEQ ID NO. 1 “HPV” and SEQ ID NO. 11 “LPEP”), HLA A2 (SEQ ID NO. 5 “GLC” and SEQ ID NO. 7 “CLG”), HLA A24 (SEQ ID NO. 10 “TYG” and SEQ ID NO. 16 “PYL”) and HLA B8 (SEQ ID NO. 4 “FLR” and SEQ ID NO. 8 “RAK”) restricted peptides, and then T cell specificity and polyfunctionality were assessed using multiparametric ICS assay, as described hereinabove.

Results

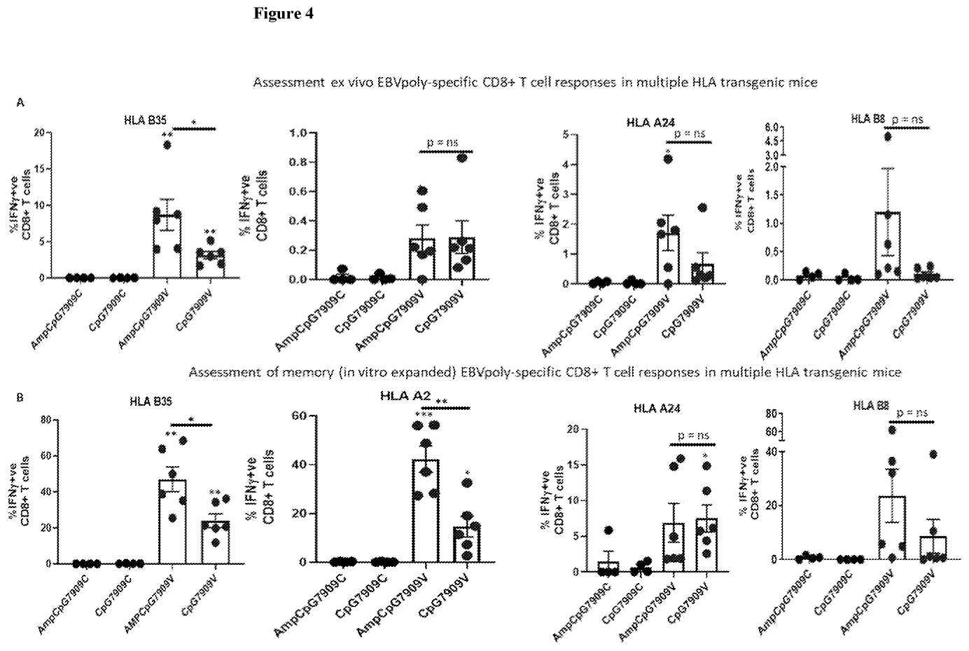

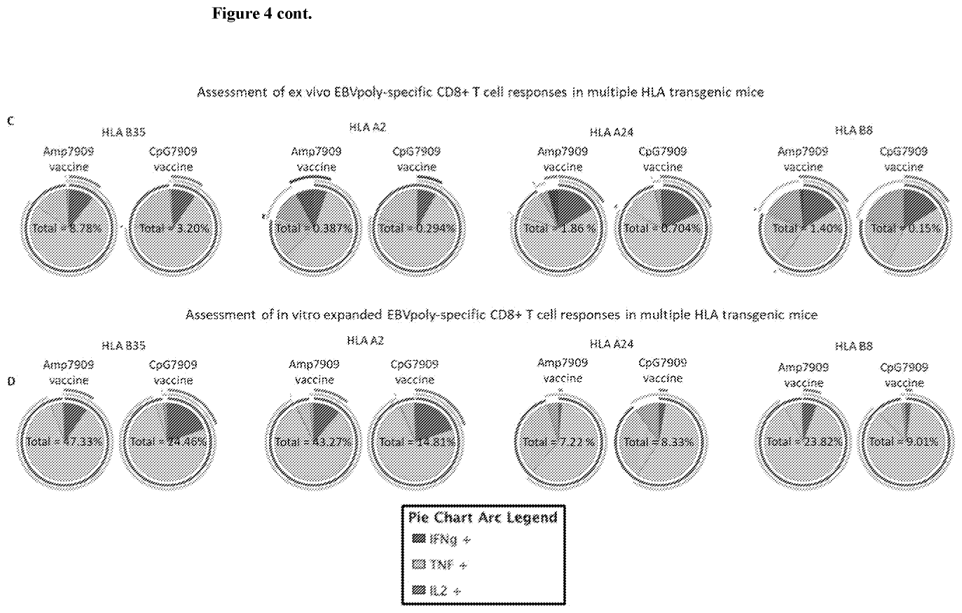

Immunization with the EBV vaccine formulated with amphCpG7909 induced a significantly greater amount of IFNγ-secreting, EBVpoly-specific CD8⁺ T cell responses in HLA B35, A24 and B8 human HLA transgenic mice ex vivo compared to EBV vaccine formulated with soluble CpG7909 or adjuvant alone control groups (See FIG. 4A). Interestingly, similar observations were noted with in vitro expanded EBVpoly-specific CD8⁺ T cell cells. The frequency of EBV-specific CD8⁺ T cells producing IFN-γ was significantly higher in HLA B35, A2 and B8 mice vaccinated with the EBV vaccine formulated with amphCpG7909 compared to EBV vaccine formulated with soluble CpG7909 formulation or adjuvant-alone control formulations. (FIG. 4B). Polyfunctional T cells play a crucial role in controlling viral infections. Thus, vaccine-induced EBV-specific CD8⁺ T cells were also assessed for their ability to secrete multiple cytokines. Notably, ex vivo analysis revealed that HLA B35, A2, A24 and B8 mice vaccinated with EBV vaccine formulated with amphCpG7909 induced greater populations of triple-positive (i.e., 3 functions; IFNγ, TNF and IL2) and double-positive (i.e., 2 functions; IFNγ and TNF) EBVpoly-specific CD8⁺ T cells compared to mice vaccinated with EBV vaccine formulated with soluble CpG7909 or adjuvant-alone controls. (See FIG. 4C). In addition, the EBV vaccine formulated with amphCpG7909 also induced higher frequencies of EBV-specific memory CD8⁺ T cell responses in HLA B35, A2, A24 and B8 mice and majority of these cells were able to produce three (IFNγ, TNF and IL2) or two (IFNγ and TNF). (See FIG. 4D).

### Example 6: Intracellular Cytokine Staining to Assess EBV Gp350-Specific CD4⁺ T Cells Producing Multiple Cytokines

As described herein (see also schematic of FIG. 3), immunized Human HLA B35, A2, A24 and B8 transgenic mice were sacrificed on day 49 and single-cell suspensions were made from splenocytes. These cells were stimulated with 0.2 μg/mL of gp350 PepMix™ EBV, a pool of 224 peptides derived from a peptide scan (15mers with 11 aa overlap) through Envelope glycoprotein GP350/GP340 (Swiss-Prot ID: P03200) of Epstein-Barr virus (HHV4) (Product Code: PM-EBV-GP350/GP340; JPT Peptide Technologies GmbH, Berlin, Germany; incorporated herein by reference), to detect EBV-specific CD4⁺ T cell responses, for four hours in vitro, in the presence of Golgiplug™ and Golgistop™ for 5 hours. Cells were washed twice, then incubated with, Live/Dead™ near IR, FITC-conjugated anti-CD4 and PerCP5.5 conjugated anti-CD8. Cells were fixed and permeabilized using a BD Cytofix/Cytoperm™ kit, then incubated with PE-conjugated anti-IFN-γ, PE-Cy7 conjugated anti-TNF, and APC conjugated anti-IL2 PE. Cells were acquired on a BD FACSCanto™ II and data was analyzed using FlowJo™ software.

To determine the EBV gp350-specific memory CD4+ T cell responses, single-cell suspensions of splenocytes derived from immunized mice, as described hereinabove, were stimulated in vitro with PepMix™ EBV to expand gp350-specific memory CD4⁺ T cells. Cultures were grown for 10 days with IL2 supplementation. On day 10 the expanded T cells were stimulated with PepMix™ EBV and T cell specificity was assessed using multiparametric ICS assay as described above.

Results

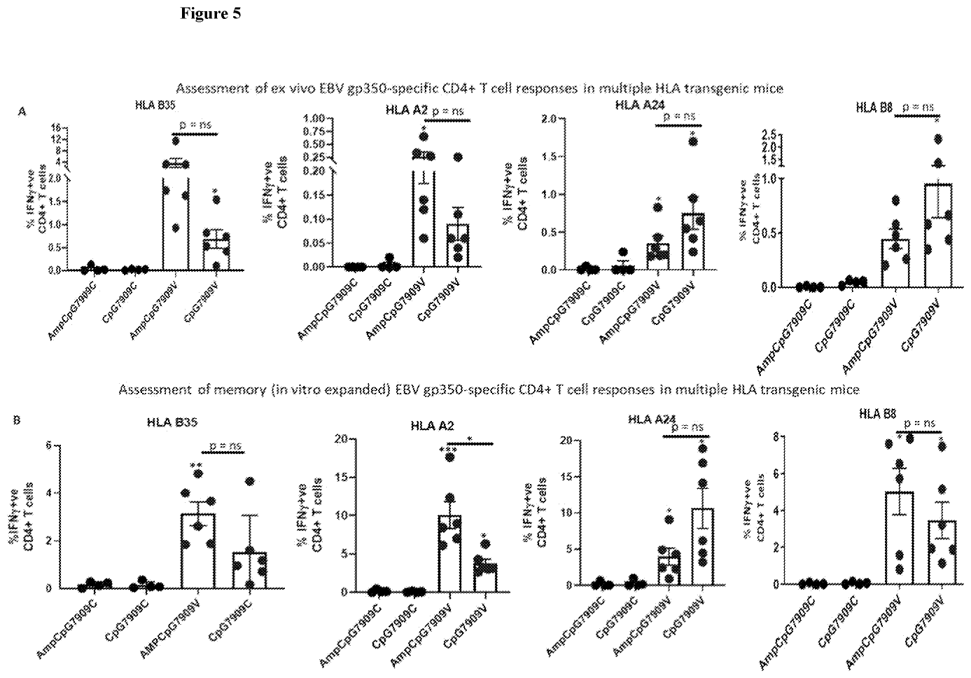

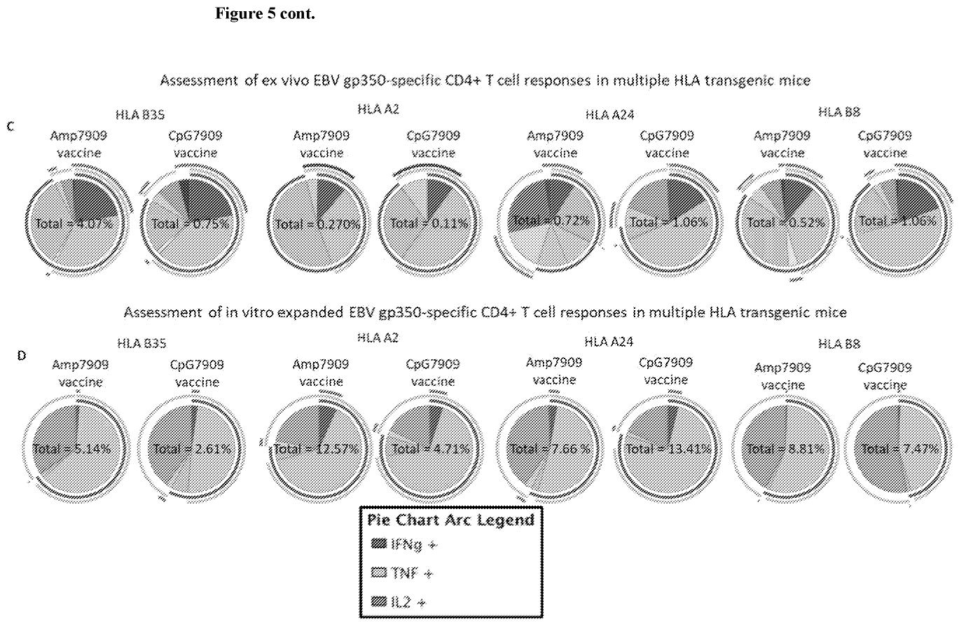

Immunization with the EBV vaccine formulated with amphCpG7909 induced higher proportion of ex vivo IFNγ secreting EBV gp350-specific CD4⁺ T cell responses in HLA B35 and A2 mice compared to EBV vaccine formulated with soluble CpG7909 or adjuvant-alone control groups, whilst EBV vaccine formulated with soluble CpG7909 induced higher proportion of ex vivo IFNγ secreting EBV gp350-specific CD4⁺ T cell responses in HLA A24 and B8 mice. (See FIG. 5A). In addition, The EBV vaccine formulated with amphCpG7909 triggered greater expansion of IFN-γ-producing EBV-specific CD4⁺ T cells in HLA B35, A2, and B8 mice compared to the EBV vaccine formulation with soluble CpG7909. However, in A24 mice EBV vaccine formulated with soluble CpG7909 triggered higher expansion of IFN-γ-producing EBV-specific CD4⁺ T cells. (See FIG. 5B). A similar trend was observed with multiple cytokine assay; HLA B35 and A2 mice immunized with EBV vaccine formulated with amphCpG7909 demonstrated higher frequencies of gp350-specific CD4⁺ T cells compared with mice immunised with EBV vaccine formulated with soluble CpG7909; however, a different trend was observed in HLA A24 and B8 mice immunised with EBV vaccine formulated with soluble CpG7909 as it induced higher frequencies of gp350-specific CD4⁺ T cells producing multiple cytokines compared to EBV vaccine formulated with amphCpG7909. Interestingly, although there is a difference in total frequencies, ex vivo multiple cytokine revealed that the majority of EBV gp350-specific CD4+ T cells from mice immunised with EBV vaccine formulated with amphCpG7909 or CpG7909 were triple positive (IFNγ, TNF and IL2) or double positive (IFNγ and TNF). (See FIG. 5C). Additionally, EBV vaccine formulated with amphCpG7909 also induced greater proportion of EBV gp350-specific memory CD4⁺ T cells in HLA B35, A2 and B8 mice, while EBV vaccine formulated with soluble CpG7909 triggered higher gp350 memory CD4⁺ T cells in HLA B8 mice. Remarkably, a larger proportion of expanded EBV gp350-specific CD4⁺ from both the formulations in HLA B35, A2, A24 and B8 mice demonstrated the ability to secrete two cytokines, IFN-γ and TNF. (See FIG. 5D).

### Example 7: Assessment of EBV Gp350-Specific CD8⁺ T Cell Responses Following In Vitro Expansion

As described herein (see also schematic of FIG. 3), immunized Human HLA B35, A2, A24 and B8 transgenic mice were sacrificed on day 49 and single-cell suspensions were made from splenocytes. These cells were stimulated with 0.2 μg/mL of gp350 PepMix™ EBV, a pool of 224 peptides derived from a peptide scan (15mers with 11 aa overlap) through Envelope glycoprotein GP350/GP340 (Swiss-Prot ID: P03200) of Epstein-Barr virus (HHV4) (Product Code: PM-EBV-GP350/GP340; JPT Peptide Technologies GmbH, Berlin, Germany; incorporated herein by reference), to detect EBV-specific CD8⁺ T cell responses, for four hours in vitro, in the presence of Golgiplug™ and Golgistop™ for 5 hours. Cells were washed twice, then incubated with, Live/Dead™ near IR, FITC-conjugated anti-CD4 and PerCP5.5 conjugated anti-CD8. Cells were fixed and permeabilized using a BD Cytofix/Cytoperm™ kit, then incubated with PE-conjugated anti-IFN-γ, PE-Cy7 conjugated anti-TNF, and APC conjugated anti-IL2 PE. Cells were acquired on a BD FACSCanto™ II and data was analyzed using FlowJo™ software.

Results

Although gp350-specific CD8⁺ T cell analysis was performed with splenocytes obtained from HLA B35, A2, A24 and B8 mice; detectable levels of gp350-specific CD8+ T cells were observed only in HLA B35 and A24 mice. Interestingly, in vitro stimulation with PepMix™ EBV resulted in expansion of gp350-specific CD8⁺ T cells from HLA B35 and A24 mice immunized with EBV-amphCpG7909 vaccine or vaccine comprising soluble CpG7909. (See FIGS. 6, A and B). However, the EBV vaccine formulation comprising soluble CpG7909 induced high frequencies of gp350-specific CD8⁺ T cells compared to EBV vaccine formulated with EBV-amphCpG7909 in HLA B35 and A24 mice. Particularly, both formulations induce a significant percentage of expanded gp350-specific CD8⁺ T cells capable of producing three (IFN-γ, IL2 and TNF) or two cytokines (IFN-γ and TNF) in HLA B35 and A24 mice. (See FIGS. 6, C and D).

### Example 8: Evaluation of EBV-Specific CD4⁺ and CD8⁺ T Cell Responses in Inguinal Lymph Node

Although analysis of EBV-specific immune responses in inguinal lymph nodes obtained from HLA B35, A2, A24 and B8 mice was intended, inguinal lymph node development was observed only in HLA B35 and A2 mice. In order to evaluate immune response in inguinal lymph nodes, single cell suspensions were made on day 49, following vaccination and sacrifice as described hereinabove. (See also schematic of FIG. 3). Cells were then stimulated with EBV HLA B35 restricted peptides (i.e., SEQ ID NO. 1 “HPV” and SEQ ID NO. 11 “LPEP”), HLA A2 (SEQ ID NO. 5 “GLC” and SEQ ID NO. 7 “CLG”) or PepMix™ EBV for four hours in vitro to test their ability to secrete IFN-γ or a combination of multiple cytokines (IFN-γ, TNF and IL2).

Results

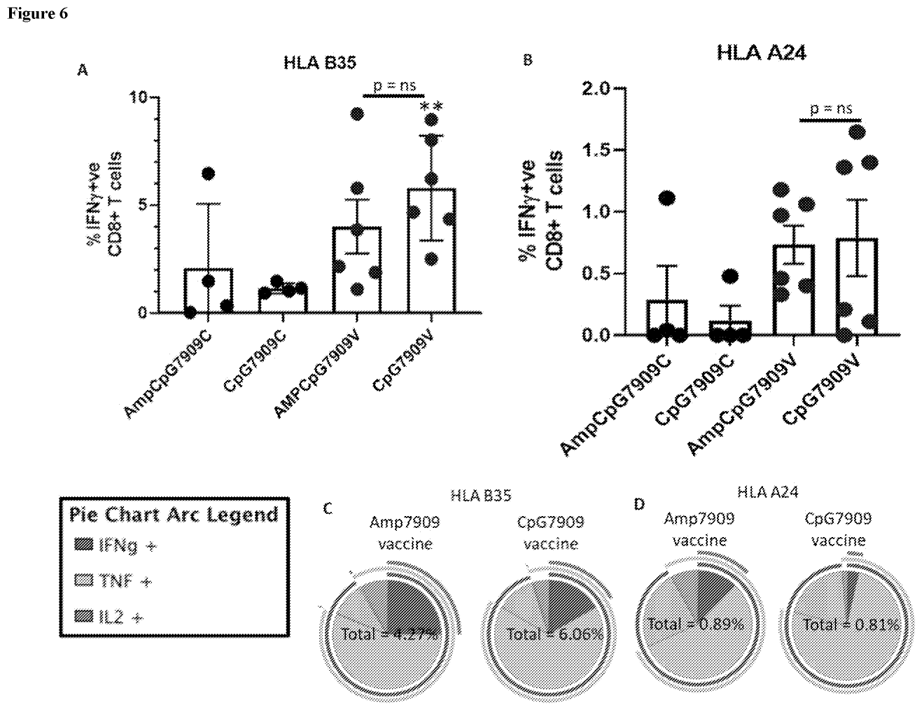

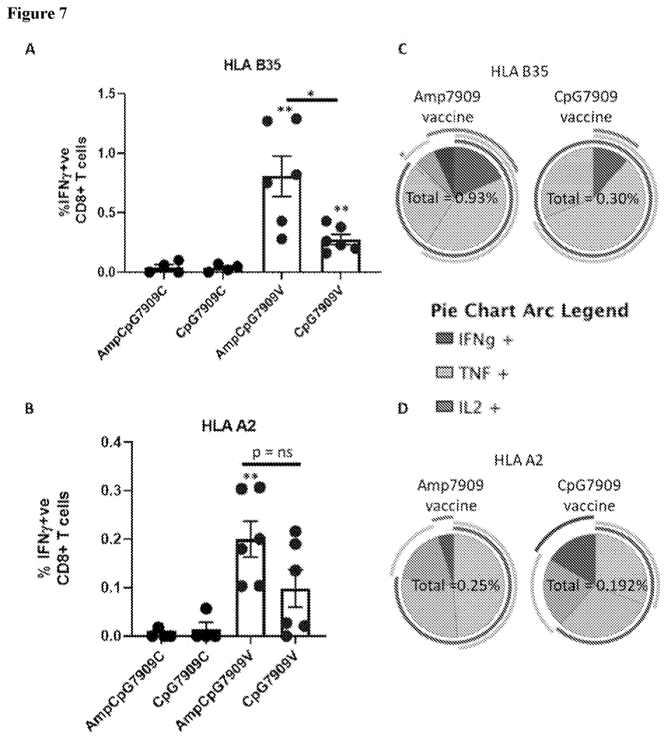

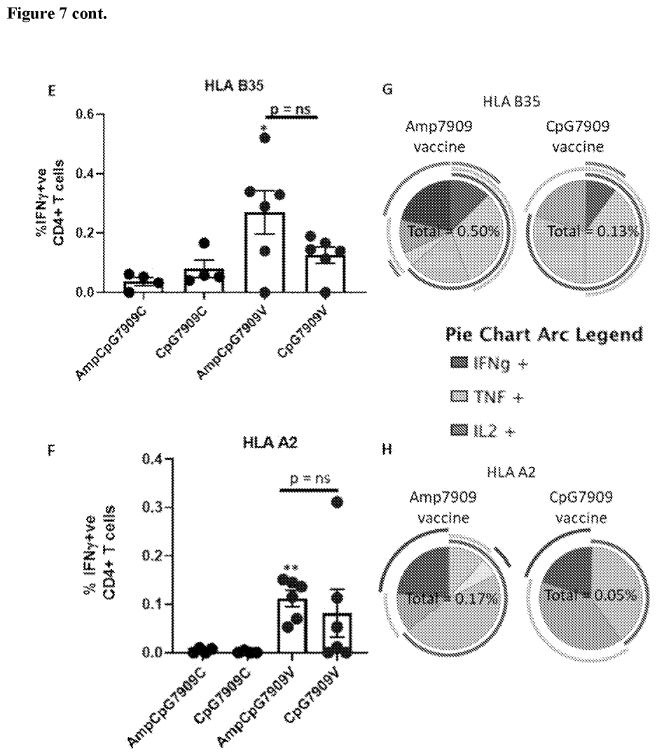

From the inguinal lymph node cells, in both HLA B35 and A2 mice the amphCpG7909 EBV vaccine induced higher frequencies of IFN-γ-producing, EBVpoly-specific, CD8⁺ T cells relative to the EBV vaccine formulated with soluble CpG7909 or the adjuvant-alone controls. (See FIGS. 7, A and B.) Notably, compared to soluble CpG7909-EBV vaccine, the amphCpG7909-EBV vaccine formulation induced higher frequencies of EBVpoly-specific CD8⁺ T cells capable of producing multiple cytokines. A large proportion of these cells were producing three (IFN-γ, TNF and IL2) or two cytokines (IFN-γ and TNF). (See FIGS. 7, C and D). The EBV vaccine formulated with amphCpG7909 also induced higher frequencies of gp350-specific CD4⁺ T cells that produced IFN-γ, compared to the EBV vaccine comprising soluble CpG7909 in HLA B35 and A2 mice. (See FIGS. 7, E and F). Further, the frequency of gp350-specific CD4⁺ T cells that produced multiple cytokines (IFN-γ and TNF) were notably higher in mice vaccinated with EBV vaccine comprising amphCpG7909 compared to the soluble CpG7909 formulation in HLA B35 and A2 mice. (See FIGS. 7, G and H.)

### Example 9: Evaluation of EBV-Specific CD4+ and CD8+ T Cells in Axillary Lymph Node

Although analysis of EBV-specific immune responses in axillary lymph nodes obtained from HLA B35, A2, A24 and B8 mice was intended, axillary lymph node development was observed only in HLA B35 and A2 mice. In order to evaluate immune response in axillary lymph nodes, single cell suspensions were made on day 49, following vaccination and sacrifice as described hereinabove. (See also schematic of FIG. 3). Cells were then stimulated with EBV HLA B35 restricted peptides (i.e., SEQ ID NO. 1 “HPV” and SEQ ID NO. 11 “LPEP”), HLA A2 (SEQ ID NO. 5 “GLC” and SEQ ID NO. 7 “CLG”) or PepMix™ EBV for four hours in vitro to test their ability to secrete IFN-γ or a combination of multiple cytokines (IFN-γ, TNF and IL2).

Results

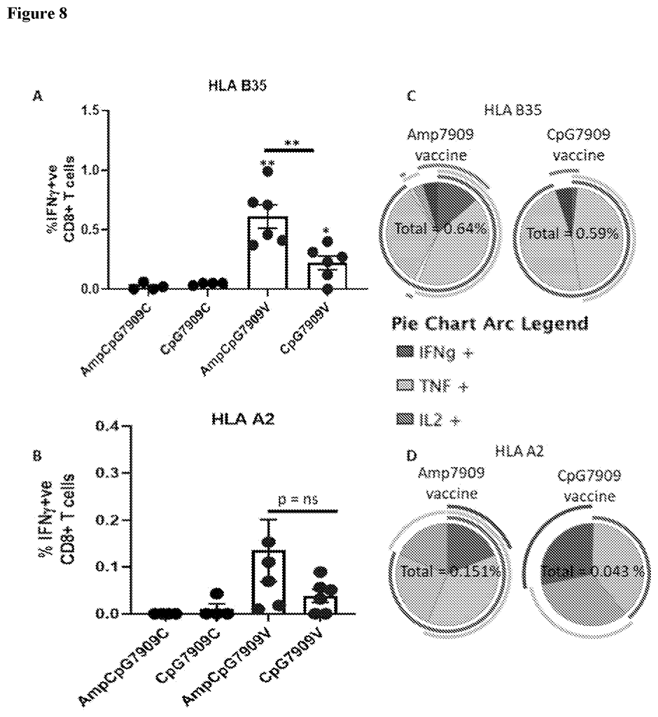

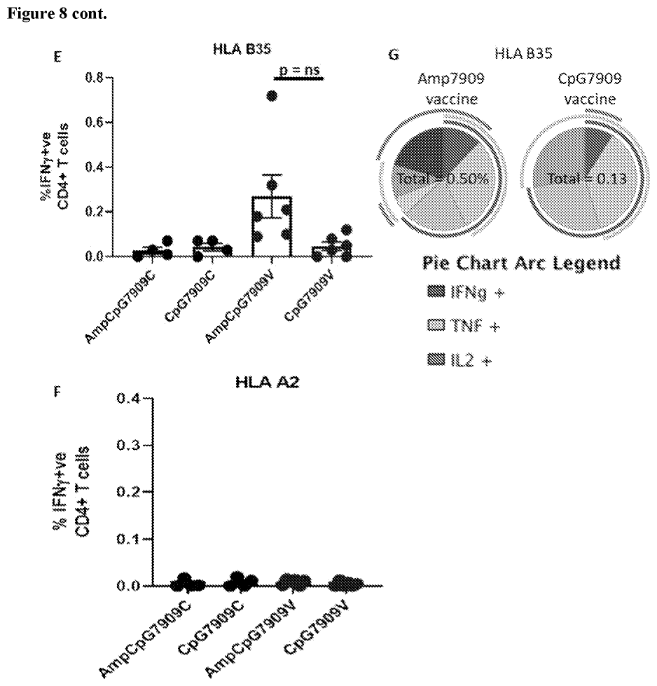

In both, HLA B35 and A2 mice, the EBV vaccine comprising amphCpG7909 induced higher frequencies of IFN-γ-producing, EBVpoly-specific, CD8⁺ T cells, compared to the soluble CpG7909, EBV vaccine or to the adjuvant-alone controls. (See FIGS. 8, A and B.) Notably, EBV vaccine comprising amphCpG7909 also boosted the ability of a large proportion of EBVpoly-specific CD8⁺ T cells to secrete two (IFN-γ and TNF) or three cytokines (IFN-γ, IL2 and TNF) in HLA B35 and A2 mice. (See FIGS. 8, C and D.) Similarly, the EBV vaccine formulated with amphCpG7909 also induced higher frequencies of gp350-specific CD4⁺ T cells producing IFN-γ compared to the soluble CpG7909, EBV vaccine formulation in HLA B35 mice; however no detectable EBV gp350-specific CD4⁺ T cells were observed in axillary lymph nodes of HLA A2 mice. (See FIGS. 8, E and F.) Additionally, the frequency of gp350-specific CD4⁺ T cells that produced three or two cytokines (IFN-γ and TNF) was remarkably higher in HLA B35 mice vaccinated with EBV vaccine formulated with amphCpG7909 compared to the soluble CpG7909 formulation. (See FIG. 8G).

Taken together, the above results indicate that EBV vaccine formulated with amphCpG7909 induced strong EBVpoly-specific CD8⁺ T cell and gp350-specific CD4⁺ T cell in spleen, and in inguinal and axillary lymph nodes, compared to EBV vaccine formulated with Soluble CpG7909.

### Example 10: Assessment of EBV Gp350-Specific Antibody Secreting B Cell Responses

Human HLA B35, A2, A24, B8 transgenic mice were immunized as outlined in FIG. 3. Upon sacrifice, splenocytes were prepared and then assessed for their ability to secrete EBV-gp350-specific antibodies using ELISpot assay.

To measure gp350-specific antibody secreting plasma B cell responses, PVDF ELISpot plates were treated with 70% ethanol. Plates were then washed five times with distilled water, coated with 100 μL/well EBV gp350 protein (25 μg/mL) or anti-IgG antibody (15 μg/mL) and incubated overnight at 4° C. Plates were blocked with DMEM containing 10% serum and 300,000 cells/well, in triplicate from each mouse, was added and then incubated for 18 hours in a 37° C. humidified incubator with 5% CO₂. Cells were removed and plates were washed. Detection antibody anti-IgG conjugated to HRP was added to each well and incubated for 2 hours at room temperature and then washed. Streptavidin-ALP was added to each well and incubated at room temperature for 1 hour, followed by washing and treating plates with substrate solution containing BCIP®/NBT (Sigma-Aldrich; MO, United States) until colour development was prominent. Colour development was stopped by washing plates with water and plates were kept for drying overnight.

To measure memory B cell response, the spleen cells (2.5×10⁴) were activated with a mixture comprising the TLR7/8-agonist, R848 (resiquimod), and recombinant mouse IL-2 for five days in 24 well plate. The ELISpot was carried out as described above. Number of spots were counted in an ELISpot reader.

Results

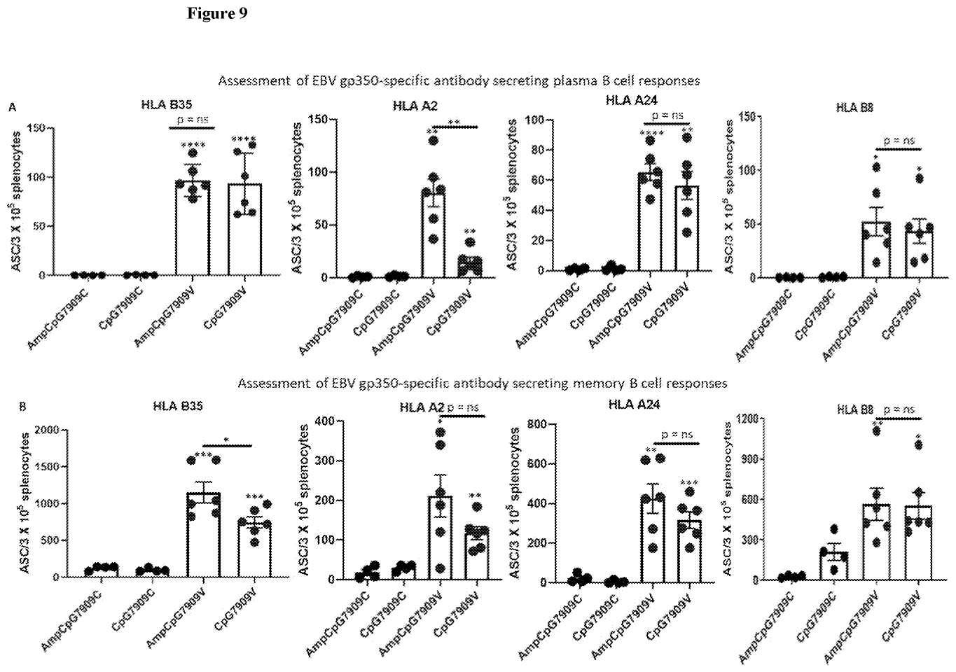

Immunization of HLA B35, A24 and B8 mice with EBV vaccine formulated with amphCpG7909 or soluble CpG7909 induced comparable levels of gp350-specific, antibody-secreting, plasma B cells; however a significant increase was observed in HLA A2 transgenic mice. (See FIG. 9A). In addition, memory B cell responses were also assessed by ex vivo polyclonal stimulation of resting B cells. Although there was no significant difference in plasma B cells, EBV vaccine formulated with amphCpG7909 vaccine induced higher frequencies of EBV-gp350-specific memory B cells. (See FIG. 9B.)

### Example 11: Assessment of EBV 213350-Specific Antibody Responses

Human HLA B35, A2, A24 and B8 transgenic mice were immunized as described hereinabove and in FIG. 3. Blood samples were collected on day 21, 28, 42 and 49 and serum was separated to assess total gp350-specific immunoglobulin (Ig) response.

Serum total anti-gp350 antibody was evaluated by an enzyme-linked immunosorbent assay (ELISA). Briefly, immunosorbent 96-well plates were coated with 50 μL of recombinant EBV gp350 protein (2.5 μg/mL of gp350 protein diluted in phosphate buffer saline) and plates were incubated at 4° C. overnight. Plates were washed with phosphate buffer saline containing Tween 20 (PBST) and then blocked with 5% skim milk. Serially diluted serum samples (day 21 or day 28) were added and incubated for 2 hours at room temperature. After washing with PBST, plates were incubated with HRP-conjugated sheep anti-mouse Ig antibody (to determine total antibody response) for 1 hour. These plates were washed and incubated with 3,3′,5,5′-Tetramethylbenzidine (TMB) substrate solution for 10 minutes and then color development was stopped by adding 1N HCl. Optical density (OD) at 450 nm was measured using an ELISA reader.

Results

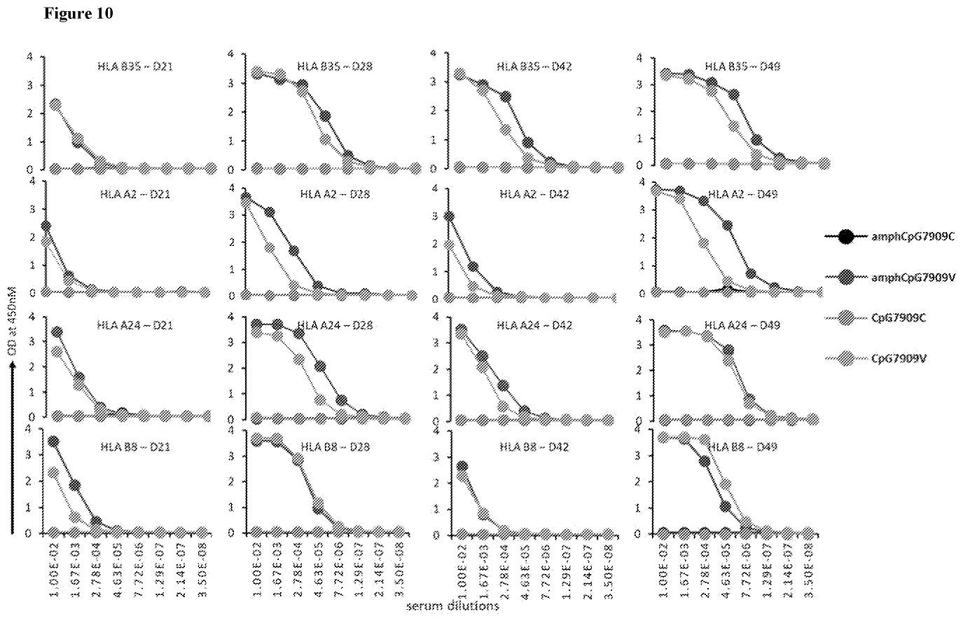

Immunization of HLA B35, A2, A24 and B8 mice with the EBV vaccine formulated with amphCpG7909 (amphCpG7909V) or soluble CpG7909 (CpG7909V) induced detectable levels gp350-specific antibody response on day 21 (single priming dose, prior to booster dose) when compared to adjuvant-alone control mice (amphCpG7909C or CpG7909C); however, antibody titres were slightly higher in HLA A2, A24 and B8 mice immunised with EBV vaccine formulated with amphCpG7909 compared to soluble CpG7909. In both the vaccinated groups, following the booster dose on days 21 and 42, EBV gp350-specific antibody titres increased significantly (day 28 and 49, respectively). Immunization with EBV vaccine formulated with amphCpG7909 resulted in higher gp350-specific antibody titres on day 28 and 49 compared to the EBV vaccine comprising soluble CpG7909 in HLA B35, A2, A24 mice, but no difference was observed in HLA B8 mice especially on day 28. In addition, there was a decreasing trend in gp350-specific antibody titres on day 42 in mice immunized with EBV vaccine formulated with either ampCpG7909 or soluble CpG7909. (See FIG. 10).

### Example 12: Assessment of EBV Gp350-Specific Antibody Isotypes

Proliferating helper T cells (i.e., CD4⁺ T cells) develop into effector T cells which differentiate into two major subtypes; T-helper type 1 and T-helper type 2 cells (Th1 and Th2 cells, respectively). Th1 cells lead to an increased cell-mediated response, the main effector cells being macrophages, CD8⁺ T cells, IgG B cells, and IFN-γ CD4⁺ T cells, and the main effector cytokines being IFN-γ and IL-2. On the other hand, Th2 cells lead to humoral immune response. The main effector cells of Th2 immunity are eosinophils, basophils, mast cells, B cells, and IL-4/IL-5 CD4 T cells; their effector cytokines being IL-4, IL-5, IL-9, IL-10, IL-13 and IL-25. In mice, Th1-dependent immunoglobulin G (IgG) subclasses include IgG2a, IgG2b, and IgG3, whereas a Th2 response stimulates the expression of IgG1. Thus, IgG subclasses can be an indicator of the underlying immune response (humoral and/or cellular).

Accordingly, serum from the immunized human HLA B35, A2, A24 and B8 transgenic mice (separated from days 21, 28, 42 and 49 blood samples) were evaluated by ELISA for antibody isotype titres and provide insight on the type of helper T cell immune response. Briefly, immunosorbent 96-well plates coated with recombinant gp350 were processed as described hereinabove, and incubated with HRP-conjugated goat anti-mouse IgA, IgM, IgG1, IgG2a, IgG2b or IgG3 antibody (to determine antibody isotype) for 1 hour. Plates were subsequently washed and incubated with TMB substrate solution for 10 minutes followed by 1N HCl and analysis using an ELISA reader.

Results

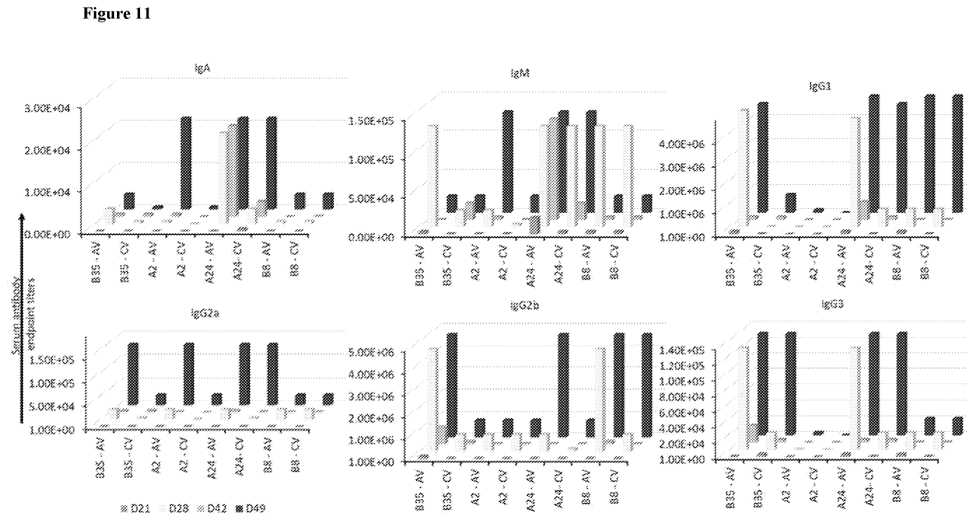

Immunization of mice with the EBV vaccine formulated with amphCpG7909 induced detectable levels of IgA on day 28 and 49 in HLA B35, A2, A24 and B8 mice, and antibody titres were clearly higher than the levels induced by the EBV vaccine formulation comprising soluble CpG7909, or adjuvant-alone controls. Similarly, on day 28 and 49 antibody isotypes IgM, IgG1, IgG2a, IgG2b and IgG3 titres were higher in mice vaccinated with amphCpG7909-EBV vaccine compared to the soluble CpG7909 formulation or the adjuvant-alone controls in HLA B35, A2, A24 and B8 mice. There was a decreasing trend in antibody isotypes titres in both the vaccine groups by day 42. The most abundant antibody isotypes were IgG2b, IgG1 and IgG3 indicating that both EBV vaccines (i.e., comprising amphCpG79090 or soluble CpG7909) have the ability to induce Th1 and Th2 type responses. (See FIG. 11).

### Example 13: Assessment of EBV-Specific Neutralizing Antibody Response

Human HLA B35, A2, A24 and B8 transgenic mice were immunized as described hereinabove and in FIG. 3. Serum separated from blood samples collected on days 21, 28, 42 and 49 were pooled to assess its ability to neutralize EBV using an EBV induced B cell proliferation assay.

Briefly, the pooled serum samples were heat inactivated at 56° C. for 30 minutes. The samples then were serially diluted in duplicates, in 2-fold dilutions (from 1:2 to 1:4096 dilution), in 25 μL volumes in a 96 well ‘U’ bottom well plate. The B95-8 isolate (virus) of EBV was added to the diluted serum samples in a 25 μL volume (50 μL/well total). The serum/virus mixture was incubated for two hours at 37° C. PBMC (100,000 cells in 50 μL/well) from EBV-seronegative donor labelled with CellTrace™ Violet (Thermo Fisher Scientific; MA, United States) was added and then incubated for one hour at 37° C. and 6.5% CO₂. Cells were washed and incubated for 5 days at 37° C. and 6.5% CO₂ to allow infection and proliferation of B cells from EBV seronegative donor. On day 5, cells were stained with Live/Dead™ near IR, APC anti-human CD3, PE-cy5 anti-human CD19. Cells were acquired on a BD FACSCanto™ II and data was analyzed using FlowJo™ software.

Results

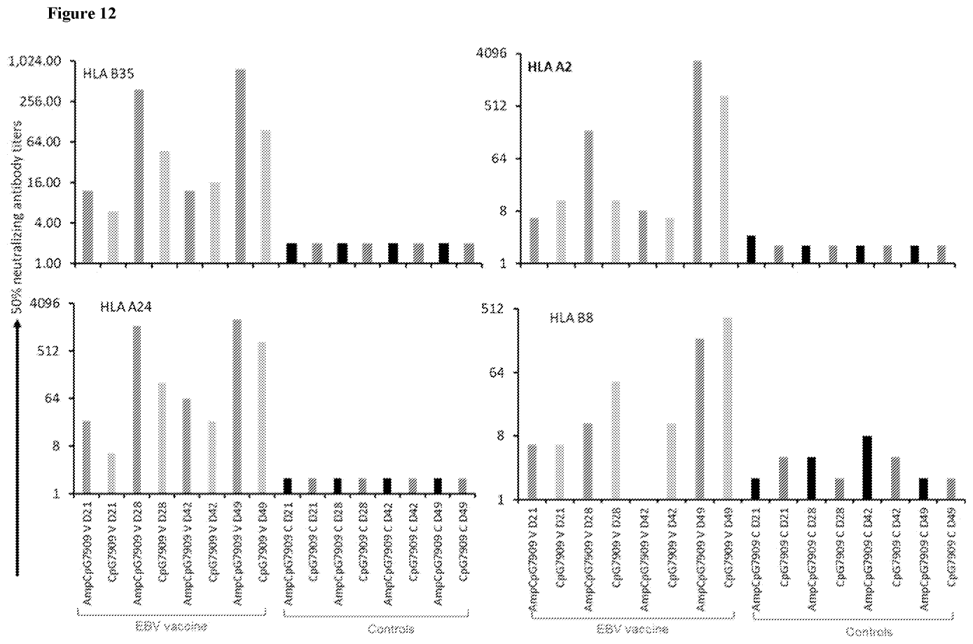

EBV neutralization assay showed that EBV vaccine formulated with amphCpG7909 clearly elicited higher anti-EBV-neutralizing antibodies on day 21, 28, and 49 compared to soluble CpG7909 formulation, or adjuvant-alone controls in HLA B35, A2 and A24 mice, while EBV vaccine formulation with soluble CpG7909 induced higher neutralizing antibody titres in HLA B8 mice. (See FIG. 12). In addition, strong gp350-specific antibody-secreting B cell response (FIG. 9), anti-gp3.50 antibody response (FIG. 10), and multiple gp350-specific antibody isotypes (FIG. 11) induced in mice immunized with amphCpG7909 correlated with neutralizing anti-EBV-neutralizing antibody titres.

### Example 14: Schematic Representation of Experimental Design for Immunogenicity Evaluation of EBV Vaccine Formulated with CpG1018 in Human HLA B35 Transgenic Mice

The adjuvant CpG1018 was recently developed and approved by the US FDA for use in human Heplisav-B® vaccine, and it is made up of cytosine phosphoguanine (CpG) motifs, which is a synthetic form of DNA that mimics bacterial and viral genetic material. CpG1018 is a 22-mer oligodeoxynucleotide with the sequence: 5′ TGA CTG TGA ACG TTC GAG ATG A 3′ (SEQ ID NO. 46). The CpG1018 adjuvant is shown to induce both humoral and cellular immune responses in various preclinical and clinical evaluation against various pathogens. Since CpG1018 is approved for human use, its ability to induce EBV-specific humoral and cellular immune responses was determined. In order to generate robust humoral and cell-mediated immune responses against EBV, the vaccine formulations were prepared by mixing EBV gp350 (10 μg) and EBVpoly protein (40 μg), with CpG1018 (50 μg) per dose in 100 μl volume. Adjuvant-alone control formulations were prepared by mixing CpG1018 (50 μg) per dose in 100 μL volume.

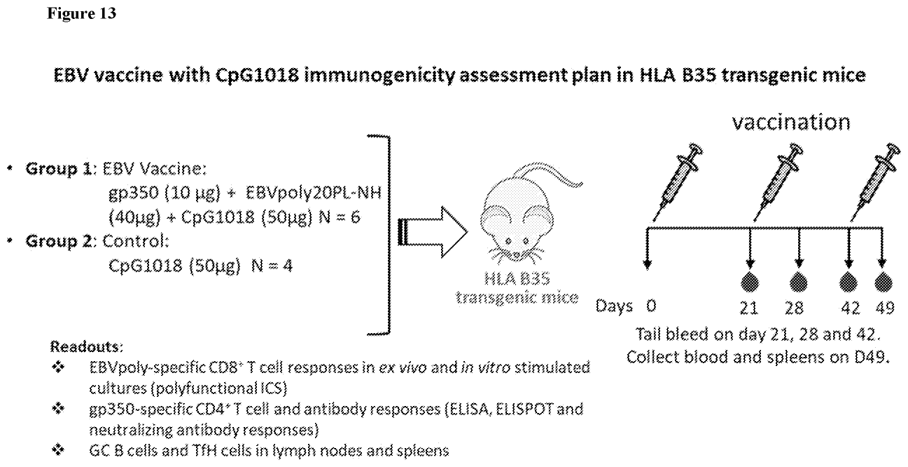

Human HLA B35 transgenic mice are deficient in expressing mouse MHC class I molecule and contain transgenes of the commonly expressed human HLA class I molecules. In order to evaluate the immunogenic response to EBV vaccine, two groups of mice were immunized with 3 doses comprising of EBVpoly and gp350 proteins, formulated with CpG1018 (EBV vaccine) or CpG1018 alone (control group). All injections (vaccine group n=6 and control group n=4) were administered subcutaneously, 100 μl at the tail base on day 0; boosted on day 21 and 42 with an identical vaccine or control formulation. The mice were tail bled on day 21, 28 and 42, and were finally sacrificed on day 49; blood and spleens were collected to assess EBV-specific humoral and cell-mediated (e.g., T cell) responses using ICS assays, gp350 ELISpot, ELISA, and neutralizing antibody assays. (See FIG. 13).

### Example 15: Intracellular Cytokine Staining to Assess EBVpolv-Specific CD8⁺ T Cells Producing Multiple Cytokines

As described herein (see also schematic of FIG. 13), immunized Human HLA B35, mice were sacrificed on day 49 and single-cell suspensions were made from splenocytes. These cells were stimulated with either 0.2 μg/mL of HLA B35 (i.e., SEQ ID NO. 1 “HPV” and SEQ ID NO. 11 “LPEP”) restricted peptides to determine the EBV-specific CD8⁺ T cell responses for four hours in vitro, in the presence of Golgiplug™ and Golgistop™ for 5 hours. Cells were washed twice, then incubated with, Live/Dead™ near IR, FITC-conjugated anti-CD4 and PerCP5.5 conjugated anti-CD8. Cells were fixed and permeabilized using a BD Cytofix/Cytoperm™ kit, then incubated with PE-conjugated anti-IFN-γ, PE-Cy7 conjugated anti-TNF, and APC conjugated anti-IL2 PE. Cells were acquired on a BD FACSCanto™ II and data was analyzed using FlowJo™ software.

To evaluate memory CD8⁺ T cell response induced following immunization with EBV vaccine formulated with CpG1018; HLA B35 splenocytes were harvested, cultures were prepared (7×10⁶ splenocytes) and stimulated in vitro with 0.211 g/mL of HLA B35 (i.e., SEQ ID NO. 1 “HPV” and SEQ ID NO. 11 “LPEP”) restricted peptides. To further expand memory EBVpoly-specific CD8⁺ T cells, cells were cultured in a 24 well plate for 10 days at 37° C., 10% CO₂, and were supplemented with IL-2 on days 2, 5 and 8. On day 10, the expanded T cells were stimulated with epitope peptides HLA B35 (i.e., SEQ ID NO. 1 “HPV” and SEQ ID NO. 11 “LPEP”) restricted peptides, and then T cell specificity and polyfunctionality were assessed using multiparametric ICS assay, as described hereinabove.

Results

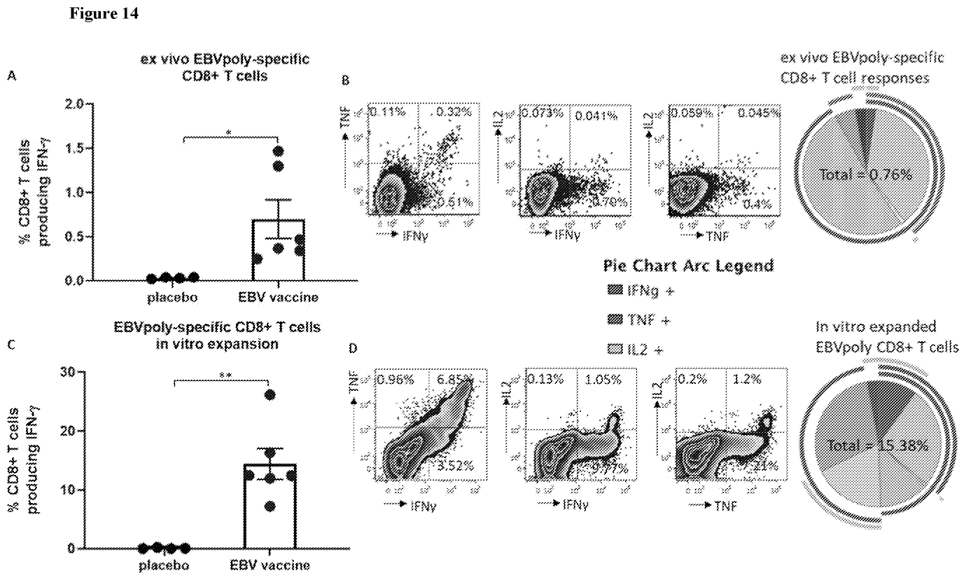

Immunization with the EBV vaccine formulated with CpG1018 induced a significantly greater amount of IFNγ-secreting, EBVpoly-specific CD8⁺ T cell responses in HLA B35 mice compared to adjuvant alone control group. (See FIG. 14A). Polyfunctional T cells play a crucial role in controlling viral infections. Thus, vaccine-induced EBV-specific CD8⁺ T cells were also assessed for their ability to secrete multiple cytokines. Notably, ex vivo analysis revealed that HLA B35 mice vaccinated with EBV vaccine formulated with CpG1018 induced greater populations of double-positive (i.e., 2 functions; IFNγ and TNF) EBVpoly-specific CD8⁺ T cells compared to mice treated with CpG1018 alone. (See FIG. 14B). In addition, the EBV vaccine formulated with CpG1018 also induced higher frequencies of EBVpoly-specific memory CD8⁺ T cell responses and majority of these cells were able to produce three (IFNγ, TNF and IL2) or two (IFNγ and TNF or TNF and IL2). (See FIGS. 14, C and D).

### Example 16: Intracellular Cytokine Staining to Assess EBV Gp350-Specific CD4⁺ T Cells Producing Multiple Cytokines

As described herein (see also schematic of FIG. 13), immunized Human HLA B35 mice were sacrificed on day 49 and single-cell suspensions were made from splenocytes. These cells were stimulated with 0.211 g/mL of gp350 PepMix™ EBV, a pool of 224 peptides derived from a peptide scan (15mers with 11 aa overlap) through Envelope glycoprotein GP350/GP340 (Swiss-Prot ID: P03200) of Epstein-Barr virus (HHV4) (Product Code: PM-EBV-GP350/GP340; JPT Peptide Technologies GmbH, Berlin, Germany; incorporated herein by reference), to detect EBV-specific CD4⁺ cell responses in vitro; in the presence of Golgiplug™ and Golgistop™ for 5 hours. Cells were washed twice, then incubated with Live/Dead™ near IR, FITC-conjugated anti-CD4 and PerCP5.5 conjugated anti-CD8. Cells were fixed and permeabilized using a BD Cytofix/Cytoperm™ kit, then incubated with PE-conjugated anti-IFN-γ, PE-Cy7 conjugated anti-TNF, and APC conjugated anti-IL2 PE. Cells were acquired on a BD FACSCanto™ II and data was analyzed using FlowJo™ software.

To determine the EBV gp350-specific memory CD4⁺ T cell responses, single-cell suspensions of splenocytes derived from immunized mice, as described hereinabove, were stimulated in vitro with PepMix™ EBV to expand gp350-specific CD4⁺ T cells. Cultures were likewise grown for 10 days with IL2 supplementation. On day 10 the expanded T cells were stimulated with PepMix™ EBV and T cell specificity was assessed using multiparametric ICS assay.

Results

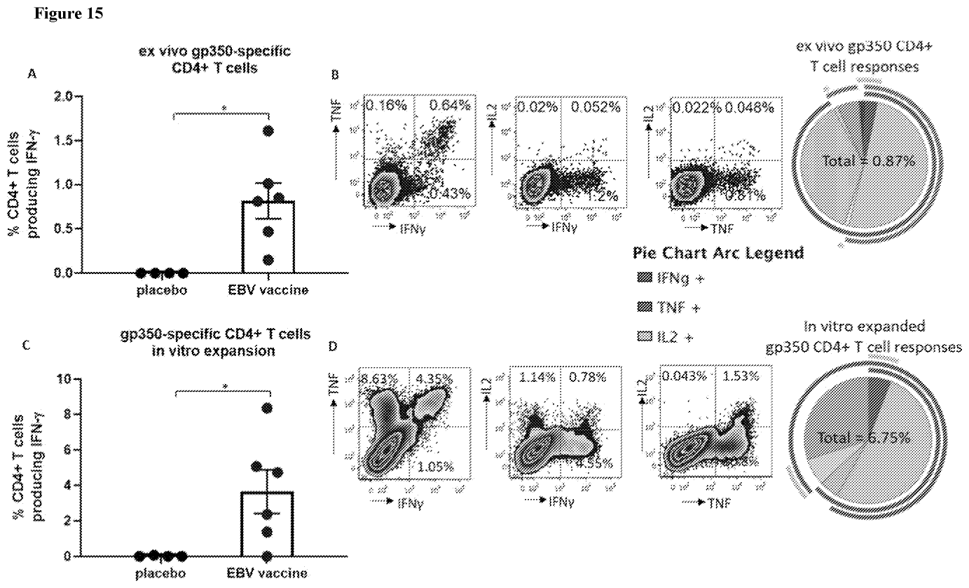

Immunization with the EBV vaccine formulated with CpG1018 induced higher proportion of ex vivo IFNγ secreting EBV gp350-specific CD4⁺ T cell responses in HLA B35 compared to CpG1018 adjuvant-alone control group. (See FIG. 15A). A similar trend was observed with multiple cytokine assay; HLA B35 mice immunized with EBV vaccine formulated with CpG1018 demonstrated higher frequencies of gp350-specific CD4⁺ T cells producing multiple cytokines compared with mice treated with CpG1018 alone, and majority of these cells were double positive were triple positive (IFNγ, TNF and IL2) or double positive (IFNγ and TNF). (See FIG. 15B). Additionally, EBV vaccine formulated with CpG1018 also induced greater proportion of EBV gp350-specific memory CD4⁺ T cells, and a larger proportion of expanded EBV gp350-specific CD4⁺ demonstrated their ability to secrete three cytokines (IFN-γ, IL2 and TNF) or two cytokines (IFN-γ and TNF or TNF and IL2). (See FIGS. 15, C and D).

### Example 17: Assessment of EBV Gp350-Specific CD8⁺ T Cell Responses Following In Vitro Expansion

As described herein (see also schematic of FIG. 13), immunized Human HLA B35, transgenic mice were sacrificed on day 49 and single-cell suspensions were made from splenocytes. These cells were stimulated with 0.2 μg/mL of gp350 PepMix™ EBV, a pool of 224 peptides derived from a peptide scan (15mers with 11 aa overlap) through Envelope glycoprotein GP350/GP340 (Swiss-Prot ID: P03200) of Epstein-Barr virus (HHV4) (Product Code: PM-EBV-GP350/GP340; JPT Peptide Technologies GmbH, Berlin, Germany; incorporated herein by reference). Cells were cultured for 10 days in the presence of IL2. To detect EBV-specific CD8⁺ T cell responses, cells were stimulated with EBV gp350 pepmix for four hours in vitro, in the presence of Golgiplug™ and Golgistop™ for 5 hours. Cells were washed twice, then incubated with, Live/Dead™ near IR, FITC-conjugated anti-CD4 and PerCP5.5 conjugated anti-CD8. Cells were fixed and permeabilized using a BD Cytofix/Cytoperm™ kit, then incubated with PE-conjugated anti-IFN-γ, PE-Cy7 conjugated anti-TNF, and APC conjugated anti-IL2 PE. Cells were acquired on a BD FACSCanto™ II and data was analyzed using FlowJo™ software.

Results

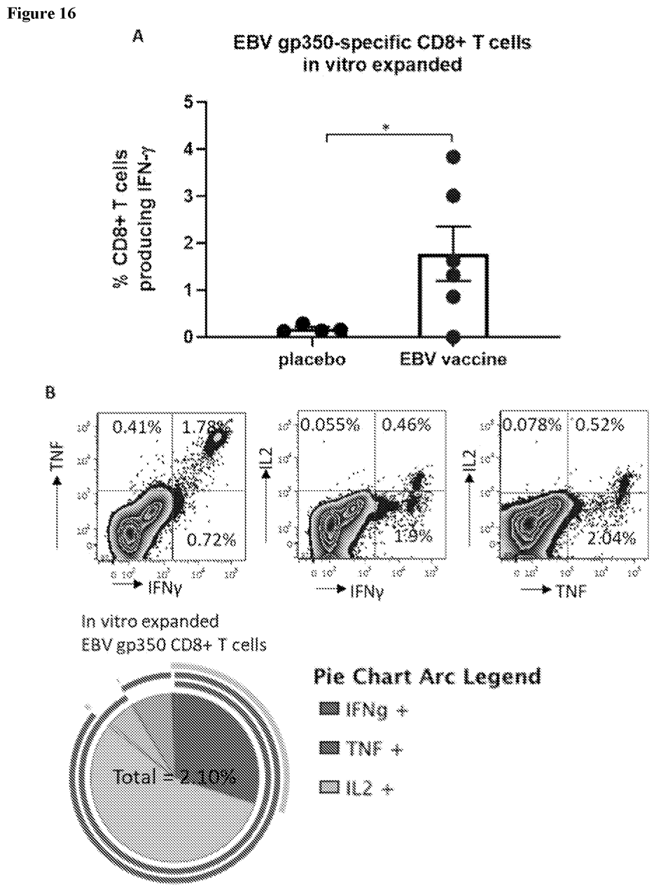

Notably, in vitro stimulation with PepMix™ EBV resulted in significantly higher expansion of IFNγ producing gp350-specific CD8⁺ T cells from HLA B35 mice immunized with EBV vaccine formulated with CpG1018 compared to mice treated with CpG1018 alone. Particularly, a significant percentage of expanded gp350-specific CD8⁺ T cells capable of producing three (IFN-γ, IL2 and TNF) or two cytokines (IFN-γ and TNF). (See FIGS. 16, A and B).

### Example 18: Assessment of Germinal Center (GC) B Cells, T_{FH} Cells and EBV Gp350-Specific Antibody Secreting B Cell Responses

Human HLA B35 transgenic mice were immunized as outlined in FIG. 13. Upon sacrifice, splenocytes were prepared and then assessed for GC B cells, T_{FH} cell responses using ICS, and EBV gp350-specific antibody secreting plasma, and memory B cells using an ELISpot assay.

To assess the GC B cell responses splenocytes were stained with PE conjugated anti-B220, FITC conjugated anti-GL7 and APC conjugated anti-CD95.

To assess T_{FH} cell responses splenocytes were stained with PerCP conjugated anti-CD8, BV786 conjugated anti-CD4 and CxCR5 and PD-1 surface markers. Cells were acquired on a BD FACSCanto II and data was analysed using FlowJo software (Tree Star).

To measure ex vivo gp350-specific antibody secreting cells, PVDF ELISpot plates were treated with 70% ethanol. Plates were then washed five times with distilled water, coated with 100 μL/well EBV gp350 protein (25 μg/mL) or anti-IgG antibody (15 μg/mL) and incubated overnight at 4° C. Plates were blocked with DMEM containing 10% serum and 300,000 cells/well, in triplicate from each mouse, was added and then incubated for 18 hours in a 37° C. humidified incubator with 5% CO₂. Cells were removed and plates were washed. Detection antibody anti-IgG conjugated to HRP was added to each well and incubated for 2 hours at room temperature and then washed. Streptavidin-ALP was added to each well and incubated at room temperature for 1 hour, followed by washing and treating plates with substrate solution containing BCIP®/NBT (Sigma-Aldrich; MO, United States) until color development was prominent. Color development was stopped by washing plates with water and plates were kept for drying overnight.

To measure memory B cell response, the spleen cells (2.5×10⁴) were activated with a mixture comprising the TLR7/8-agonist, R848 (resiquimod), and recombinant mouse IL-2 for five days in 24 well plate. The ELISpot was carried out as described above. Number of spots were counted in an ELISpot reader.

Results

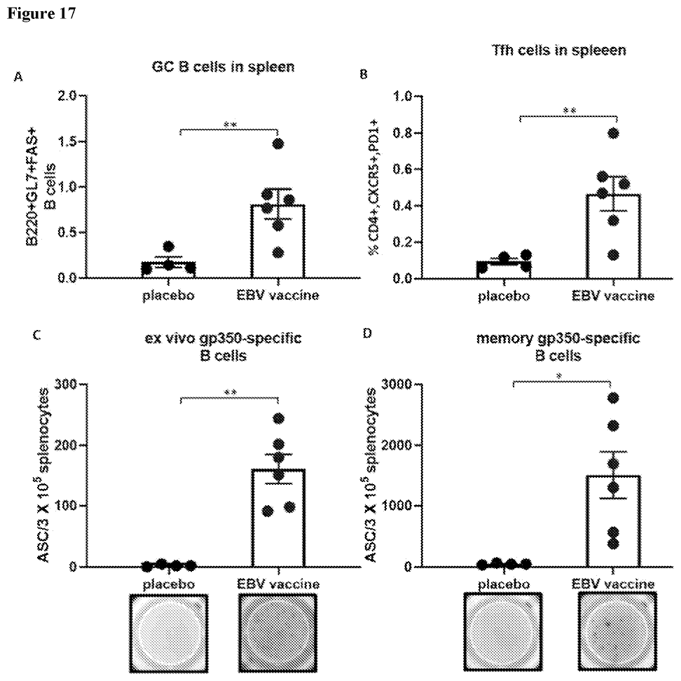

The assessment of GC B and T_{FH} cell responses in spleen indicated that EBV vaccine formulated with CpG1018 induced significantly higher frequencies of GC B and T_{FH} cell responses compared to mice treated with CpG1018 alone. (FIGS. 17, A and B).

Immunization of HLA B35 mice with EBV vaccine formulated with CpG1018 induced significantly higher levels of gp350-specific, antibody-secreting, plasma and memory B cells responses compared to placebo group mice. (See FIGS. 17, C and D).

### Example 19: Assessment of EBV Gp350-Specific Antibody Isotypes

Serum from the immunized human HLA B35 transgenic mice (separated from days 21, 28, 42 and 49 blood samples) was evaluated by ELISA for antibody isotype titres, and to provide insight on the type of helper T cell immune response. Briefly, immunosorbent 96-well plates coated with recombinant gp350 were processed as described hereinabove, and incubated with HRP-conjugated goat anti-mouse IgA, IgM, IgG1, IgG2a, IgG2b or IgG3 antibody (to determine antibody isotype) for 1 hour. Plates were subsequently washed and incubated with TMB substrate solution for 10 minutes followed by 1N HCl and analysis using an ELISA reader.

Results

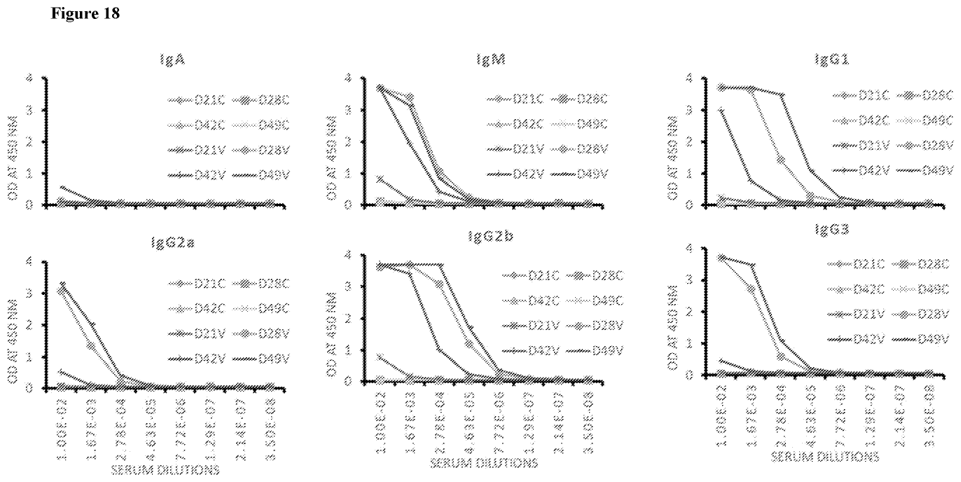

Immunization of mice with the EBV vaccine formulated with CpG108 induced detectable levels of IgA on day 49. In addition, following booster dose on day 21 and 42, on day 28 and 49 antibody isotypes IgM, IgG1, IgG2a, IgG2b and IgG3 titres were higher in mice vaccinated with CpG1018-EBV vaccine compared to placebo group. The most abundant antibody isotypes were IgG2b, IgG1 and IgG3 indicating that EBV vaccine with CpG1018 has the ability to induce Th1 and Th2 type responses. (See FIG. 18).

### Example 20: Assessment of EBV-Specific Neutralizing Antibody Response

Human HLA B35 transgenic mice were immunized as described hereinabove and in FIG. 13. Serum separated from blood samples collected on days 21, 28, 42 and 49 were pooled to assess its ability to neutralize EBV using an EBV induced B cell proliferation assay.

Briefly, the pooled serum samples were heat inactivated at 56° C. for 30 minutes. The samples then were serially diluted in duplicates, in 2-fold dilutions (from 1:2 to 1:4096 dilution), in 25 μL volumes in a 96 well ‘U’ bottom well plate. The B95-8 isolate (virus) of EBV was added to the diluted serum samples in a 25 μL volume (50 μL/well total). The serum/virus mixture was incubated for two hours at 37° C. PBMC (100,000 cells in 50 μL/well) from EBV-seronegative donor labelled with CellTrace™ Violet (Thermo Fisher Scientific; MA, United States) was added and then incubated for one hour at 37° C. and 6.5% CO₂. Cells were washed and incubated for 5 days at 37° C. and 6.5% CO₂ to allow infection and proliferation of B cells from EBV seronegative donor. On day 5, cells were stained with Live/Dead™ near IR, APC anti-human CD3, PE-cy5 anti-human CD19. Cells were acquired on a BD FACSCanto™ II and data was analyzed using FlowJo™ software.

Results

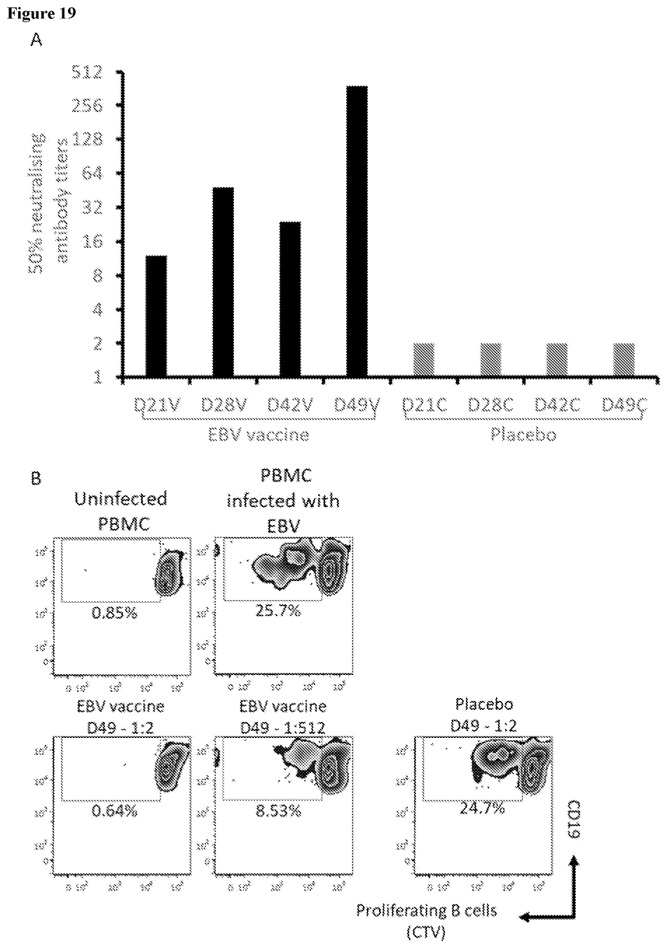

EBV neutralization assay showed that EBV vaccine formulated with CpG1018 clearly induced higher anti-EBV-neutralizing antibodies on day 21, 28, and 49 compared to adjuvant-alone control. However, following the booster dose of days 21 and 42, on day 28 and 49 the EBV vaccine formulated with CpG1018 induced a 4- and 32-fold increase in EBV neutralizing antibody titers, respectively (see FIGS. 19, A and B).

All publications, patents, patent applications and sequence accession numbers mentioned herein are hereby incorporated by reference in their entirety as if each individual publication, patent or patent application was specifically and individually indicated to be incorporated by reference. In case of conflict, the present application, including any definitions herein, will control.

Those skilled in the art will recognize, or be able to ascertain using no more than routine experimentation, many equivalents to the specific embodiments of the invention described herein. Such equivalents are intended to be encompassed by the following claims.

What is claimed is:

1. An immunogenic polypeptide comprising amino acid sequences of each of a plurality of cytotoxic T-cell (CTL) epitopes from herpesvirus antigens, wherein the polyepitope protein further comprises proteasome liberation amino acids or amino acid sequences between at least two of said plurality of CTL epitopes and wherein the polyepitope protein is capable of eliciting a CTL response upon administration to a subject as an exogenous polypeptide,
wherein the polypeptide comprises at least one of the CTL epitope amino acid sequences set forth in SEQ ID NOs. 1-20.

2. The immunogenic polypeptide of claim 1, wherein the proteasome liberation amino acids or amino acid sequences comprise AD, K and/or R.

3. The immunogenic polypeptide of claim 1, further comprising at least one of the CTL epitope amino acid sequences set forth in SEQ ID NOs. 1-20, or combinations thereof.

4. The immunogenic polypeptide of claim 1, comprising each of the CTL epitope amino acid sequences set forth in SEQ ID NOs. 1-20.

5. The immunogenic polypeptide of any one of claims 1-4, comprising the amino acid sequence set forth in SEQ ID NO. 21.

6. The immunogenic polypeptide of any one of claims 1-5, wherein each of the epitopes are restricted by any one of the HLA class I specificities selected from HLA A*03, HLA All, HLA A*0201, HLA A*1101, HLA A*2301, HLA A*3002, HLA B27, HLA B35.08/B35.01, HLA B*44:0, HLA B57*03, HLA B*0702, HLA B*0801, HLA B*1501, HLA B*3501, HLA B*3508, HLA B*4001, HLA B*4402, HLA B*4402, HLA B*4403, HLA B*4405, HLA B*5301, HLA B*5701, or HLA B*5801.

7. The immunogenic polypeptide of claim 6, wherein the epitopes are derived from any one of the Epstein-Barr virus (EBV) antigens EBNA1, EBNA3A, EBNA3B, EBNA3C, LMP2, LMP2a, BMLF1, BZLF1, or BRLF1.

8. A pharmaceutical composition comprising one or more of the immunogenic polypeptides of any one of claims 1-7, further comprising one or more immunogenic glycoproteins, or fragments thereof.

9. The pharmaceutical composition of claim 8, wherein the immunogenic glycoproteins are derived from herpesvirus.

10. The pharmaceutical composition of claim 9, wherein the immunogenic glycoproteins are derived from EBV.

11. The pharmaceutical composition of claim 10, wherein the immunogenic glycoproteins comprise at least one of glycoprotein 350 (gp350), glycoprotein B (gB), glycoprotein H (gH), glycoprotein (gL), gHgL complex, glycoprotein 42 (gp42), any fragment thereof, or any combination thereof.

12. The pharmaceutical composition of claim 11, wherein the immunogenic glycoprotein comprises gp350, or any fragment thereof.

13. The pharmaceutical composition of any one of claims 1 to 11, further comprising one or more adjuvants.

14. The pharmaceutical composition of claim 13, wherein the adjuvant comprises at least one of a toll-like receptor (TLR) agonist, a cationic anti-microbial peptide (CAMP), Adjuvant α-GalCer, aluminum phosphate, aluminum hydroxide, calcium phosphate, β-Glucan Peptide, CpG DNA, GPI-0100, lipid A, monophosphorylated lipid A (MPL), lipopolysaccharide, Lipovant, Montanide, N-acetyl-muramyl-L-alanyl-D-isoglutamine, Pam3CSK4, quil A, or trehalose dimycolate.

15. The pharmaceutical composition of claim 14, wherein the adjuvant comprises a TLR9 agonist.

16. The pharmaceutical composition of claim 14 or 15, wherein the adjuvant comprises an oligodeoxynucleotide (ODN).

17. The pharmaceutical composition of 16, wherein the adjuvant is a CpG ODN.

18. The pharmaceutical composition of any one of claims 14 to 17, wherein the adjuvant is an amphiphilic CpG ODN.

19. The pharmaceutical composition of any one of claims 8 to 17, comprising gp350, and further comprising a CpG ODN adjuvant.

20. A multivalent vaccine, comprising the pharmaceutical composition of any one of claims 8 to 19.

21. An isolated nucleic acid encoding the immunogenic polypeptide of any one of the preceding claims.

22. The isolated nucleic acid of claim 21, comprising a nucleic acid sequence selected from SEQ ID NOs. 22-41.

23. An expression vector comprising the isolated nucleic acid of any one of claim 21 or 22 operably linked to one or more regulatory sequences.

24. A host cell comprising the expression vector of claim 23.

25. A method for producing the immunogenic polypeptide of any one of claims 1 to 7, wherein said method includes steps for purifying the immunogenic polypeptide under conditions that maintain the immunogenic polypeptide in a substantially non-aggregated form.

26. A prophylactic or therapeutic composition for eliciting an immunogenic response in a subject against a herpesvirus, the composition comprising:
i. an immunogenic polypeptide comprising amino acid sequences derived from each of a plurality of cytotoxic T-cell (CTL) epitopes, wherein the polypeptide comprises the amino acid sequences set forth in SEQ ID NOs. 1 and 11;
ii. at least one herpesvirus glycoprotein; and
iii. at least one adjuvant.

27. The composition of claim 26, wherein the immunogenic polypeptide further comprises at least one of the CTL epitope amino acid sequences set forth in SEQ ID NOs. 12-20.

28. The composition of claim 26, wherein the immunogenic polypeptide comprises each of the CTL epitope amino acid sequences set forth in SEQ ID NOs. 1-20.

29. The composition of any one of claims 26 to 28, wherein the immunogenic polypeptide comprises the amino acid sequence set forth in SEQ ID NO. 21.

30. The composition of any one of claims 26 to 29, wherein each of the epitopes of the immunogenic polypeptide are restricted by any one of the HLA class I specificities selected from HLA A*03, HLA A11, HLA A*0201, HLA A*1101, HLA A*2301, HLA A*3002, HLA B27, HLA B35.08/B35.01, HLA B*44:0, HLA B57*03, HLA B*0702, HLA B*0801, HLA B*1501, HLA B*3501, HLA B*3508, HLA B*4001, HLA B*4402, HLA B*4402, HLA B*4403, HLA B*4405, HLA B*5301, HLA B*5701, or HLA B*5801.

31. The composition of any one of claims 26 to 30, wherein each of the epitopes of the immunogenic polypeptide are derived from any one of EBV antigens EBNA1, EBNA3A, EBNA3B, EBNA3C, LMP2, LMP2a, BMLF1, BZLF1, or BRLF1.

32. The composition of claim 26, wherein the glycoprotein is derived from EBV.

33. The composition of claim 32, comprising at least one of gp350, gB, gH, gL, gHgL complex, gp42, any fragment thereof, or any combination thereof.

34. The composition of claim 26, wherein the adjuvant comprises a TLR agonist.

35. The composition of claim 34, wherein the TLR agonist comprises an ODN.

36. The composition of claim 34 or 35, wherein the wherein the adjuvant is a CpG ODN.

37. The composition of claim 36, wherein the adjuvant is a CpG ODN conjugated to a lipid.

38. A multivalent EBV vaccine comprising:
i. an immunogenic polypeptide as set forth in SEQ ID NO. 21;
ii. at least one EBV glycoprotein; and
iii. at least one adjuvant.

39. The multivalent EBV vaccine of claim 38, wherein the EBV glycoprotein is selected from at least one of gp350, gB, gH, gL, gHgL complex, gp42, any fragment thereof, or any combination thereof.

40. The multivalent EBV vaccine of claim 38, wherein the EBV glycoprotein is gp350, or a fragment thereof.

41. The multivalent EBV vaccine of any one of claims 38 to 40, wherein the adjuvant comprises a TLR agonist.

42. The multivalent EBV vaccine of claim 41, wherein the adjuvant is a CpG ODN.

43. The multivalent EBV vaccine of claim 42, wherein the adjuvant is a CpG ODN conjugated to a lipid.

44. A method for generating a prophylactic or therapeutic treatment for herpesvirus infection comprising combining an isolated immunogenic polypeptide, at least one herpesvirus glycoprotein, at least one adjuvant comprising a TLR agonist, and a pharmaceutically acceptable excipient, in a formulation suitable for administration to a subject;
wherein the immunogenic polypeptide comprises at least one of the CTL epitope amino acid sequences set forth in SEQ ID NOs. 1 and 11.

45. The method of claim 44, wherein the immunogenic polypeptide is encoded by a nucleic acid comprising at least one of the nucleic acid sequences set forth in SEQ ID NOs. 22-41.

46. The method of claim 44, wherein the immunogenic polypeptide is encoded by a nucleic acid comprising each of the nucleic acid sequences set forth in SEQ ID NOs. 22-41.

47. The method of claim 44, wherein the immunogenic polypeptide is encoded by a nucleic acid comprising the nucleic acid sequence set forth in SEQ ID NO. 42.

48. The method of any one of claims 44 to 47, wherein the immunogenic polypeptide comprises the amino acid sequence set forth in SEQ ID NO. 21.

49. The method of any one of claims 44 to 48, wherein the herpesvirus glycoprotein is derived from EBV.

50. The method of claim 49, wherein the herpesvirus glycoprotein comprises at least one of gp350, gB, gH, gL, gHgL complex, gp42, any fragment thereof, or any combination thereof.

51. The method of any one of claims 44 to 50, wherein the adjuvant comprises a TLR9 agonist.

52. The method of any one of claims 44 to 51, wherein the adjuvant comprises an ODN.

53. The method of any one of claims 44 to 52, wherein the adjuvant is a CpG ODN.

54. The method of any one of claims 44 to 53, wherein the adjuvant is a CpG ODN conjugated to a lipid.

55. A method for prophylactically or therapeutically treating a herpesvirus infection in a subject, comprising administering to the subject a composition comprising:
i. an immunogenic polypeptide comprising amino acid sequences derived from each of a plurality of cytotoxic T-cell (CTL) epitopes, wherein the polypeptide comprises the amino acid sequences set forth in SEQ ID NOs. 1 and 11;
ii. at least one herpesvirus glycoprotein;
iii. and an adjuvant.

56. The method of claim 55, wherein the immunogenic polypeptide further comprises at least one of the CTL epitope amino acid sequences set forth in SEQ ID NOs. 1-20, or combinations thereof.

57. The method of claim 55, wherein the immunogenic polypeptide comprises each of the CTL epitope amino acid sequences set forth in SEQ ID NOs. 1-20.

58. The method of any one of claims 55 to 57, wherein the immunogenic polypeptide comprises the amino acid sequence set forth in SEQ ID NO. 21.

59. The method of any one of claims 55 to 58, wherein each of the epitopes are restricted by any one of the HLA class I specificities selected from HLA class I specificities selected from HLA A*03, HLA A11, HLA A*0201, HLA A*1101, HLA A*2301, HLA A*3002, HLA B27, HLA B35.08/B35.01, HLA B*44:0, HLA B57*03, HLA B*0702, HLA B*0801, HLA B*1501, HLA B*3501, HLA B*3508, HLA B*4001, HLA B*4402, HLA B*4402, HLA B*4403, HLA B*4405, HLA B*5301, HLA B*5701, or HLA B*5801.

60. The method of any one of claims 55 to 59, wherein the epitopes are derived from any one of EBV antigens EBNA1, EBNA3A, EBNA3B, EBNA3C, LMP2, LMP2a, BMLF1, BZLF1, or BRLF1.

61. The method of any one of claims 58 to 60, comprising 20-50 μg of the immunogenic polypeptide.

62. The method of claim 61, comprising 40 μg of the immunogenic polypeptide.

63. The method of any one of claims 55 to 62, wherein the glycoprotein is selected from gp350, gB, gH, gL, gHgL complex, gp42, any fragment thereof, or any combination thereof.

64. The method of any one of claims 55 to 63, wherein the glycoprotein is gp350, or a fragment thereof.

65. The method of any one of claims 55 to 64, wherein the adjuvant comprises a TLR agonist.

66. The method of any one of claims 55 to 65, wherein the adjuvant comprises an oligodeoxynucleotide (ODN).

67. The method of any one of claims 55 to 66, wherein the adjuvant is a CpG ODN.

68. The method of any one of claims 55 to 67, wherein the adjuvant is a CpG ODN conjugated to a lipid.

69. A method of inducing proliferation of herpesvirus-specific CTLs, comprising bringing a sample comprising CTLs into contact with one or more peptides comprising CTL epitope amino acid sequences set forth in SEQ ID NOs. 1-20, or combinations thereof.

70. The method of claim 69, comprising bringing the sample into contact with a pool of peptides comprising at least one of the CTL epitope amino acid sequences set forth in SEQ ID NOs. 1-20, or combinations thereof.

71. The method of claim 69, comprising bringing the sample into contact with a pool of peptides comprising each of the CTL epitope amino acid sequences set forth in SEQ ID NOs. 1-20.

72. The method of claim 69, comprising incubating a sample comprising CTLs with antigen-presenting cells (APCs) that present at least one peptide comprising at least one of the CTL epitope amino acid sequences set forth in SEQ ID NOs. 1-20.

73. The method of claim 72, wherein the APCs present a plurality of peptides comprising at least one of the CTL epitope amino acid sequences set forth in SEQ ID NOs. 1-20, or combinations thereof.

74. The method of claim 72 or 73, wherein the APCs present a plurality of peptides comprising each of the CTL epitope amino acid sequences set forth in SEQ ID NOs. 1-20.

75. An antigen-presenting cell (APC) comprising a peptide of any one of claims 1 to 7 presented on a class I MHC.

76. A method of producing an APC that presents one or more EBV peptides comprising incubating an antigen-presenting cell with the one or more peptides of any one of claims 1 to 7 or one or more nucleic acids encoding the one or more peptides of any one of claims 1 to 7.
